(19) United States
(12) Patent Application Publication
(10) Pub. No.: US 2010/0010079 A1
(43) Pub. Date: Jan. 14, 2010

(54) Proanthocyanidins for the Treatment of Amyloid and Alpha-Synuclein Diseases

(76) Inventors: Alan D. Snow, Lynnwood, WA (US); Gerardo M. Castillo, Bothell, WA (US); Paula Y. Choi, Seattle, WA (US); Beth P. Nguyen, Gurnee, IL (US)

Correspondence Address: JONES DAY, 222 EAST 41ST ST, NEW YORK, NY, 10017, US

(21) Appl. No.: 12/470,374

(22) Filed: May 21, 2009

Related U.S. Application Data
(63) Continuation of application No. 10/077,596, filed on Feb. 15, 2002, now abandoned.
Application No. 10/077,596 is a continuation-in-part of application No. 10/053,625, filed on Nov. 2, 2001, now Pat. No. 6,929,808.
Application No. 10/053,625 is a continuation-in-part of application No. 09/753,313, filed on Dec. 29, 2000, now abandoned.
Application No. 09/753,313 is a continuation-in-part of application No. 09/938,987, filed on Aug. 24, 2001, now Pat. No. 6,607,758.
Application No. 09/938,987 is a continuation of application No. 09/079,829, filed on May 15, 1998, now Pat. No. 6,939,570.
(60) Provisional application No. 60/046,672, filed on May 16, 1997.
(60) Provisional application No. 60/338,721, filed on Dec. 4, 2001.
(60) Provisional application No. 60/339,033, filed on Dec. 10, 2001.
(60) Provisional application No. 60/276,866, filed on Mar. 15, 2001.
(60) Provisional application No. 60/338,969, filed on Dec. 10, 2001.

(51) Int. Cl. A61K 31/352; A61K 31/357; A61P 25/28

(52) U.S. Cl. 514/456; 514/453

(57) ABSTRACT

A method of treating an amyloid disease, or a disease characterized by α-synuclein or NAC fibrillogenesis, in a mammalian subject. The method includes administering to the mammal a therapeutically effective amount of a various disclosed proanthocyanidins or a proanthocyanidin characterized by disclosed general formulae. A pharmaceutical composition comprising a therapeutically effective amount of a proanthocyanidin and a pharmaceutically acceptable excipient. The therapeutic amount of the proanthocyanidin is selected for efficacy in treating amyloid, α-synuclein or NAC fibrillogenesis in a mammalian subject.

The instant application claims priority under 35 U.S.C. § 120 to, and is a continuation of, U.S. application Ser. No. 10/077,596, which is a Continuation-in-pan of U.S. Ser. No. 10/053,625 filed Nov. 21, 2001. This is also a Continuation-in-pan of U.S. Ser. No. 09/753,313 filed Dec. 29, 2000, and a Continuation-in-part of U.S. Ser. No. 09/938,987 filed Aug. 24, 2001, which is a continuation of Ser. No. 09/107,9829 filed May 15, 1998, now abandoned, which claimed priority to U.S. provisional application 60/046,602 filed May 15, 1997. This application also claims priority to U.S. provisional applications 60/338,721 filed Dec. 4, 2001, 60/339,033 filed Dec. 10, 2001, 60/276,866 filed Mar. 15, 2001 and 60/338,969 filed Dec. 10, 2001.

### NOTICE REGARDING FEDERAL FUNDING

This invention was made with government support under 2 R44AG16551-02 awarded by the National Institute on Aging. The Government may have certain rights in the invention.

### TECHNICAL FIELD

The invention relates to methods and compositions for treatment and prevention of amyloid, NAC (i.e. non-amyloid component) and α-synuclein diseases, such as Alzheimer's disease and Parkinson's disease, and to method of isolation of new compounds for the same; particularly it relates to polyphenolic compositions and methods of using same to treat these same diseases; more particularly it relates to proanthocyanidin and related compounds for treatment and prevention of amyloid, NAC and α-synuclein diseases.

### BACKGROUND OF THE INVENTION

Alzheimer's disease is characterized by the accumulation of a 39-43 amino acid peptide termed the beta-amyloid protein or AB, in a fibrillar form, existing as extracellular amyloid plaques and as amyloid within the walls of cerebral blood vessels. Fibrillar AB amyloid deposition in Alzheimer's disease is believed to be detrimental to the patient and eventually leads to toxicity and neuronal cell death, characteristic hallmarks of Alzheimer's disease. Accumulating evidence implicates amyloid, and more specifically, the formation, deposition, accumulation and/or persistence of AB fibrils, as a major causative factor of Alzheimer's disease pathogenesis. In addition, besides Alzheimer's disease, a number of other amyloid diseases involve accumulation of AC fibrils, including Down's syndrome, disorders involving congophilic angiopathy, hereditary cerebral hemorrhage of the Dutch type, and inclusion body myositosis.

Parkinson's disease is another human disorder characterized by the formation, deposition, accumulation and/or persistence of abnormal fibrillar protein deposits that demonstrate many of the characteristics of amyloid. In Parkinson's disease, an accumulation of cytoplasmic Lewy bodies consisting of filaments of α-synuclein/NAC are believed important in the pathogenesis and as therapeutic targets. New agents or compounds able to inhibit α-synuclein/NAC formation, deposition, accumulation and/or persistence, or disrupt pre-formed α-synuclein/NAC fibrils (or portions thereof) are regarded as potential therapeutics for the treatment of Parkinson's disease.

A variety of other human diseases also demonstrate amyloid deposition and usually involve systemic organs (i.e. organs or tissues lying outside the central nervous system), with the amyloid accumulation leading to organ dysfunction or failure. These amyloid diseases (discussed below) leading to marked amyloid accumulation in a number of different organs and tissues are known as systemic amyloidoses. In other amyloid diseases, single organs may be affected such as the pancreas in 90% of patients with type 2 diabetes. In this type of amyloidosis, the beta-cells in the islets of Langerhans in pancreas are believed to be destroyed by the accumulation of fibrillar amyloid deposits consisting primarily of a protein known as islet amyloid polypeptide (IAPP). Inhibiting or reducing such amyloid accumulation is believed to lead to new effective treatments for type 2 diabetes. In Alzheimer's disease, Parkinson's and “systemic” amyloid diseases, there is currently no cure or effective treatment, and the patient usually dies within 3 to 10 years from disease onset.

The amyloid diseases include, but are not limited to, the amyloid associated with Alzheimer's disease, Down's syndrome, hereditary cerebral hemorrhage with amyloidosis of the Dutch type, and inclusion body myositosis (Askanas et al, Ann. Neurol. 43:521-560, 1993) (wherein the specific amyloid is referred to as beta-amyloid protein or AB), the amyloid associated with chronic inflammation, various forms of malignancy and Familial Mediterranean Fever (wherein the specific amyloid is referred to as AA amyloid or inflammation-associated amyloidosis), the amyloid associated with multiple myeloma and other β-cell dyscrasias (wherein the specific amyloid is referred to as AL amyloid), the amyloid associated with type II diabetes (wherein the specific amyloid protein is referred to as amylin or islet amyloid polypeptide), the amyloid associated with the prion diseases including Creutzfeldt-Jakob disease, Gerstmann-Straussler syndrome, kuru and animal scrapie (wherein the specific amyloid is referred to as PrP amyloid), the amyloid associated with long-term hemodialysis and carpal tunnel syndrome (wherein the specific amyloid is referred to as beta₂-microglobulin amyloid), the amyloid associated with senile cardiac amyloid and Familial Amyloidotic Polyneuropathy (wherein the specific amyloid is referred to as transthyretin or prealbumin), and the amyloid associated with endocrine tumors such as medullary carcinoma of the thyroid (wherein the specific amyloid is referred to as variants of procalcitonin). In addition, the α-synuclein protein which forms fibrils, and is Congo red and Thioflavin S positive, is found as part of lewy bodies in the brains of patients with Parkinson's disease, Lewy body disease (Lewy in Handbuch der Neurologie, M. Lewandowski, ed., Springer, Berline pp. 920-933, 1912; Pollanen et al, J. Neuropath. Exp. Neurol. 52:183-191, 1993; Spillantini et al, Proc. Natl. Acad. Sci. USA 95:6469-6473, 1998; Arai et al, Neurosc. Lett. 259:83-86, 1999), and multiple system atrophy (Wakabayashi et al, Acta Neuropath. 96:445-452, 1998). For purposes of this disclosure, Parkinson's disease, due to the fact that fibrils develop in the brains of patients with this disease (which are Congo red and Thioflavin S positive, and which contain predominant beta-pleated sheet secondary structure), should be regarded as a disease that also displays the characteristics of an amyloid-like disease.

Discovery and identification of new compounds or agents as potential therapeutics to arrest amyloid formation, deposition, accumulation and/or persistence that occurs in Alzheimer's disease, Parkinson's disease, type II diabetes, systemic AA amyloidosis, and other amyloidoses are desperately sought.

Polyphenols are an incredibly diverse group of compounds (Ferreira et al, Tetrahedron 48:1743-1803, 1992) that widely occur in a variety of plants, some of which enter into our food chain. Although some of the polyphenols are considered to be nonnutritive, interest in these compounds has arisen because of their possible beneficial effects for health. For example, quercetin (a flavanoid) has been shown to possess anticarcinogenic activity in experimental studies (Kato et al, Carcinogenesis 4:1301-1305, 1983; Deschner et al, Carcinogenesis 7:1193-1196, 1991). Catechin and epicatechin (flavan-3-ols) have been shown to inhibit Leukemia virus reverse transcriptase activity (Chu et al, J. Natural Prods. 55: 179-183, 1992). Statistical reports have shown that stomach cancer mortality is significantly lower in the tea producing districts of Japan. Epigallocatechin gallate has been reported to be the pharmacologically active material in green tea that inhibits mouse skin tumors (Mimoto et al, Carcinogenesis 21:915-919, 2000). Ellagic acid has also been shown to possess anticarcinogenic activity in various animal tumor models (Inoue et al, Biol. Pharm Bull. 18:1526-1530, 1995). However, none of the literature teaches or suggests that proanthocyanidins, and procyanidins, particularly epicatechin-epicatechin dimers or trimers or other oligomers, epicatechin-catechin dimers or the like, or analogs or derivatives thereof, have any benefit for the inhibition of amyloid or α-synuclein/NAC fibril formation, and/or cause a disruption of pre-formed amyloid or α-synuclein/AC fibrils.

### DISCLOSURE OF THE INVENTION

Methods pertaining to the isolation, identification and use of anti-amyloid compounds derived from plant material, and the surprising discovery that proanthocyanidins are potent inhibitors of amyloid and α-synuclein/NAC fibrillogenesis, and cause a potent disruption/disassembly of pre-formed fibrils for a variety of amyloid and α-synuclein diseases are disclosed. Exemplary compounds are identified to serve as potent amyloid fibril inhibiting agents, including procyanidins, such as epicatechin-epicatechin, catechin-epicatechin dimers, epiafzelechin-epicatechin dimers, epicatechin-epicatechin-epicatechin trimers, as well as other epicatechin and/or catechin oligomers for the treatment of amyloid diseases including, but not limited to, Alzheimer's disease, type II diabetes, and systemic AA amyloidosis, as well as inhibiting α-synuclein or non-amyloid component (NAC) fibril formation for the treatment of Parkinson's and Lewy body disease.

Also disclosed are methods for preparing and isolating such compounds, as well as new uses for them, especially as amyloid and α-synuclein/NAC fibril disrupting agents. This invention is also directed to methods for inhibiting or eliminating amyloid fibril formation, deposition, accumulation and/or persistence in a number of different amyloid diseases by treatment of patients with proanthocyanidins, such as procyanidins of the A, B and C types, or other monomers, dimers, trimers and multimers of epicatechin and catechin. Exemplary compounds are substituted epicatechin-epicatechin or catechin-epicatechin dimers, such as epicatechin-4β-epicatechin or catechin-4α→8-epicatechin, and epiafzelechin-4β→8-epicatechin, or other proanthocyanidin oligomers.

Also disclosed are methods of isolation, identification and use of amyloid-inhibiting compounds derived from plant material for the therapeutic intervention of Alzheimer's disease, type II diabetes, Parkinson's disease, systemic AA amyloidosis and other disorders involving amyloid fibril accumulation; more particularly, it relates to methods of isolating amyloid-inhibiting compounds from Uncaria tomentosa and related plants, and other known proanthocyanidin producing plants, and to the use of those compounds.

A surprising discovery is noted that specific extraction methods (and individual compounds derived from such extraction methods) when applied to the inner bark and root parts of Uncaria tomentosa, otherwise known as Uña de Gato (or Cat's claw), lead to the isolation and purification of single compounds (such as “compound H2” identified as an epicatechin-epicatechin dimer; “compound H1” identified as a catechin-epicatechin dimer, “compound K2” identified as an epicatechin-epicatechin-epicatechin trimer), and “compound K1” identified as a epiafzelechin-epicatechin dimer, all of which act as impressive inhibitors of Alzheimer's disease beta-amyloid protein (AB) formation and growth, Parkinson's disease α-synuclein fibril formation and growth, and causes disruption/dissolution of pre-formed Alzheimer's, Parkinson's and type II diabetes fibrils.

Previously our studies have led to the identification of a natural substance derived from the Amazon rain forest woody vine, Uncaria tomentosa, and referred to as PTI-00703. See for instance U.S. patent application Ser. Nos. 09/079,829, 09/198,824, and 09/208,278, which describe the initial discovery of derivatives of Uncaria tomentosa and related plant material extracts as inhibitors of amyloidosis of Alzheimer's disease, type II diabetes and other amyloid disorders, the disclosures of which are hereby incorporated by reference as if fully set forth. This was followed up by the parent application to this case, the disclosures of which are hereby incorporated by reference as if fully set forth, which used assay-guided affinity fractionation and reverse phase high pressure liquid chromatography (HPLC) methodology to isolate, test and characterize the most active water-soluble ingredients within PTI-00703 (collectively referred to as “PTI-777”) that appear to account for the majority of the Aβ fibrillogenesis inhibitory activity.

In these latter disclosures, it is discussed how PI-777 and its individual fractions as isolated by HLC were tested in relevant in vitro and/or animal models, and found to consistently demonstrate inhibition of Aβ fibrillogenesis. Also described were extraction methods for the isolation of PTI-777 and its individual fractions and/or components. Further purification and in vitro testing of each of the PTI-777 compounds, as well as initial structural characterization studies suggested that the amyloid inhibitor compounds derived from Uncaria tomentosa are small molecules (˜200-500 molecular weight) that belong to the general class of aromatic polyphenolic compounds. Two such compounds, chlorogenic acid (C₁₆H₁₈O₉; FW 354.31)(earlier referred to as “Fraction F”) and epicatechin (C₁₅H₁₄O₆; FW 290.27)(earlier referred to as “Fraction J”) were purified and identified by analytical techniques. In addition, data indicates that “fraction H” isolated from PTI-777 was a most potent inhibitor of amyloid fibrillogenesis. In addition, PTI-777 has the ability to enter the brain as demonstrated by radiolabelling experiments, indicating that it has the potential to be useful as a therapeutic agent for Alzheimer's disease, Parkinson's disease, and other central nervous system disorders involving deposition and accumulation of fibrillar proteins, such as type 2 diabetes and systemic AA amyloidosis.

We have now further purified, isolated and identified additional major components of PTI-777, and demonstrated a further surprising discovery that such single compounds (which belong to the general class of proanthocyanidins) are potent amyloid inhibiting agents. Compound H2, by mass spectroscopy studies, was shown to be a major component of PTI-777, and purified, and finally identified (as described herein) as epicatechin-4β→8-epicatechin, also known as procyanidin B2. Compound H1, also a major component of PTI-777, was purified and identified (as described herein) as catechin-4α→8-epicatechin, also known as procyanidin B4. Compound K2, a component of PTI-777, was purified and identified as epicatechin-4β→8-epicatechin-4β→8-epicatechin, also known as procyanidin C1. Compound K1, a component of PTI-777, was purified and identified as epiafzelechin-4β→8-epicatechin.

Efficacy of each of these proanthocyanidins as a potent inhibitor of Alzheimer's AB amyloidosis, Parkinson's disease α-synuclein/NAC fibrillogenesis, and type II diabetes IAPP fibrillogenesis, is disclosed herein, and supports the conclusion that procyanidins in particular, and proanthocyanidins in general, are useful compounds for the treatment of amyloidosis and related fibrillogenesis associated with Alzheimer's disease, Parkinson's disease, type 2 diabetes, systemic AA amyloidosis and other amyloid diseases.

A method of treating a human or other a mammal suffering from, or subject to, an amyloid disease, or any disease characterized by α-synuclein or NAC fibrillogenesis is disclosed. Any mammal may be the subject or the disease or condition, or simply be a mammal that is subject to the disease or condition. Amyloid disease as used herein includes but is not limited to the various known and disclosed amyloidoses discussed herein. A disclosed treatment for an amyloid disease is intended to cover a like treatment for the corresponding amyloidosis, and vice-versa. The same is true for α-synuclein diseases. The term “fibrillogenesis” refers to the fibril, plaque and tangle-like forming propensities of the various substituent proteins and/or precursor proteins disclosed herein, whether or not any particular degree of fibrillogenesis has progressed, or is expected to progress, to any particular recognized amyloidosis or to an amyloid or α-synuclein disease. In general, treatment of fibrillogenesis as disclosed is intended to include and cover treatment of any amyloidosis or any amyloid or α-synuclein disease corresponding to, following from, otherwise related to, that fibrillogenesis.

“Treatment” is also intended in every possible instance to include and cover “in vitro treatment”, whether for experimental or screening purposes and the like, and whether or not the in vitro treatment leads to, or is ever intended to lead to, treatment of a like fibrillogenesis, or any amyloid or α-synuclein disease corresponding to that fibrillogenesis, in a mammalian subject.

The method includes administering to the mammal a therapeutically effective amount of any proanthocyanidin, or proanthocyanidin compound, that may be found in the group of proanthocyanidin and proanthocyanidin compounds characterized by either Formula I or Formula II, or both (see FIGS. 54-56). The group also includes proanthocyanidins characterized by oligomeric combinations of Formula I and Formula II (see FIG. 56), and also includes any pharmaceutically acceptable salt of any of the foregoing proanthocyanidins.

As discussed in more detail elsewhere herein, proanthocyanidins (also referred to herein as PA) include a variety of structural shapes and oligomeric forms. Formulae I and II are intended each to represent one general form of an oligomeric unit effective to make up the various disclosed oligomers. For instance some proanthocyanidin oligomers are well characterized by Formula I, which is to say that the general structure stated by Formula I is a valid generalization of each unit of the oligomer. An example of a proanthocyanidin characterized by Formula I is epicatechin-4β→8-epicatechin, a dimer where two epicatechin units, each a particular instance of, and conforming to, Formula I, are joined from the 4 carbon atom of one unit, to the 8 carbon atom of the other unit, thus effecting a so-called 4-8 linkage. In like manner, proanthocyanidin oligomers entirely characterized by Formula II will have all their units joined from the 4 carbon atom of one unit to the 6 carbon atom of the adjacent unit, thus effecting a so-called 4-6 linkage, and so on.

Some proanthocyanidins can not be well characterized by only Formula I or Formula II, in fact presenting in one unit a Formula I configuration and in another unit a Formula II configuration. For instance, a proanthocyanidin having a unit in a 4-6 linkage to a second unit which itself has a 4-8 linkage to a third unit is not strictly either a Formula I or a Formula II compound, but is actually an oligomeric combination of Formula I and Formula II, where among other characteristics, each unit may be susceptible of more than one characterization (viz. Formula I or Formula II). In the example just given, the middle of the three units has both it's 4 carbon and 6 carbon link sites filled and is therefore a Formula II unit (it may not be possible to specify with particularity what the first unit is, since as terminal unit it has only one carbon linked); the third unit however has it's 8 carbon link site filled and whether or not it is a terminal unit, it will most likely conform to Formula I (except in the relatively rare occurrence of the third unit itself linking at it's 6 carbon site to a fourth unit, making it a unit with it's 6 and 8 carbon link sites filled—which fits neither formula, though it may be nonetheless a useful compound for treatment). Thus, an oligomer with some units 4-6 (or 6-4) linked and some 4-8 (or 8-4 linked) will show units in both Formula I and Formula II configurations, but be neither a Formula I nor a Formula II compound, but will be instead an oligomeric combination of Formula I and Formula II.

Based upon observations discussed herein, it is believed that proanthocyanidins characterized as above, or elsewhere herein, particularly with oligomer units numbering in the range of 2-20 (that is, where n in either Formula is an integer value from 2 to 20) will all be efficacious to some significant degree for treating the diseases and conditions disclosed herein. It is believed as well, though not discussed to any extent, and also based upon observations discussed herein, that proanthocyanidins not precisely adhering to the above formulaic prescriptions (such as the presence of one or more variant linkages, like the aforementioned 6-8 or 8-6 unit linkage, or even a 6-6 unit linkage) will also be efficacious Lao some significant degree for treating the diseases and conditions disclosed herein.

In the formulae shown, R₁ and R₂ are independently selected from hydrogen and hydroxy; R₃ is selected from the group consisting of hydrogen, optionally substituted O-glycosyl, —C(O)-(optionally substituted aryl), and —C(O)-(optionally substituted heteroaryl); and R₄ is selected from the group consisting of hydrogen, catechin, epicatechin, epiafzelechin, and gallates of catechin and epicatechin. The lines at the 2-, 3- and 4-position denote optional R and S (sometimes and alternatively referred to as α and β) stereochemical configurations. Generally the configuration at the 4-position is trans to the configuration at the 3-position. The lines at the 4- and 8-positions in Formula I and at the 4- and 6-positions in Formula II denote possible oligomer bonds between individual units as earlier discussed, and the each of the substitutions at R₁, R₂, R₃, and R₄, and each of the configurations at the 2-, 3-, and 4-positions, and each of the oligomer bond configurations of 4-8 and 4-6 are independently selected for each individual unit and may be different for unit in the oligomer series of units, though often the units of shorter oligomers are homogenous with one another.

Preferred oligomers with have from 2 to 5 units, or even 2-3 units. The chiral configuration at each 2-carbon position is preferably R as opposed to S. In some embodiments some or all of the units have an R₃ that is either hydrogen, 2,3-dihydroxybenzoyl, 3,4-dihydroxybenzoyl; 2,3,4-trihydroxybenzoyl, or 3,4,5-trihydroxybenzoyl, and preferably each R₃ is hydrogen and each R₁ is hydroxy and each R₂ is hydrogen. R₃ may also be an optionally substituted O-glycosyl.

Another method of treatment of an amyloid disease, or a disease characterized by α-synuclein or NAC fibrillogenesis, in a mammalian subject, is also disclosed. The method includes the step of administering to the subject a therapeutically effective amount of a proanthocyanidin. The proanthocyanidin is preferably a procyanidin oligomer having from 2 to 20, and more preferably 2-5, flavanoid units. Each flavanoid unit can advantageously be one of the catechins, including catechin, epicatechin, epiafzelechin, gallocatechin, galloepicatechin, epigallocatechin and the gallates of the catechins. The flavanoid unit can also be one of the flavanols, flavonols, flavandiols, leucocyanidins, or anthocyanidins.

The particular amyloid disease to be treated can be Alzheimer's disease, Down's syndrome, hereditary cerebral hemorrhage with amyloidosis of the Dutch type, inclusion body myositosis, the amyloidosis of chronic inflammation, the amyloidosis of malignancy and Familial Mediterranean Fever, the amyloidosis of multiple myeloma and β-cell dyscraisa, the amyloidosis of type 2 diabetes, the amyloidosis of prion diseases, Creutzfeldt-Jakob disease, Gerstmann-Straussler syndrome, kuru, scrapie, mad cow disease, the amyloidosis associated with long-term hemodialysis, the amyloidosis with carpal tunnel syndrome, senile cardiac amyloidosis, Familial Amyloidotic Polyneuropathy, the amyloidosis associated with endocrine tumors, systemic AA amyloidosis, AL amyloidosis, AB amyloidosis or PrP amyloidosis, but particularly Alzheimer's disease.

The particular α-synuclein or NAC fibrillogenesis to be treated can be the fibrillogenesis associated with Lewy body disease, Parkinson's disease or multiple system atrophy.

A method of treatment of amyloid, α-synuclein or NAC fibrillogenesis, in an in vitro environment, is also disclosed. The method includes the step of administering into the in vitro environment a therapeutically effective amount of a proanthocyanidin. Preferably the proanthocyanidin is a procyanidin which is an oligomer of any or all of epicatechin, catechin, epiafzelechin, epicatechin gallates or catechin gallates.

The procyanidin may advantageously be a procyanidin that is an A, B or C type procyanidin. The procyanidin is preferably a dimer or trimer of epicatechin and/or catechin units, such as the dimers of the type B1, B2, B3, B4, B5, B6, B7, and B8 type procyanidins. In one embodiment, the procyanidin dimer is epicatechin-4β→8-epicatechin; in another embodiment, the procyanidin dimer is catechin-4α→8-epicatechin; in still another, the procyanidin is the epicatechin trimer epicatechin-4β→8-epicatechin-4β→8-epicatechin; in yet another embodiment the procyanidin is the dimer epiafzelechin-4β→8-epicatechin.

The method may also include an administration step to deliver the procyanidin to the subject by way of oral administration, parenteral injection, intraperitoneal injection, intravenous injection, subcutaneous injection, intramuscular injection, topical administration, or aerosol spray administration.

A pharmaceutical composition or agent is also disclosed. It is a therapeutically effective amount of a proanthocyanidin (PA) together with a pharmaceutically acceptable carrier, diluent, or excipient, or the like. The therapeutic amount of the PA is selected for efficacy in treating an amyloid, α-synuclein or NAC fibrillogenesis in a mammalian subject. Disclosed compositions are delivered in therapeutic dosages in the range of about 10 mg/kg to 1,000 mg/kg of body weight of the subject, and preferably in the range of about 10 mg/kg to 100 mg/kg of body weight of the subject.

The proanthocyanidin is preferably epicatechin or one or more of the dimers and trimers of epicatechin and catechin, or a mixture thereof, as well as the pharmaceutically acceptable analogs and derivatives thereof. Preferred proanthocyanidins are the procyanidin dimer epicatechin-4β→8-epicatechin, the procyanidin dimer catechin-4α→8-epicatechin, the procyanidin dimer epiafzelechin-4β→8-epicatechin, and the procyanidin trimer epicatechin-4β→8-epicatechin-4β→8-epicatechin.

When the composition is a mixture of two or more proanthocyanidins, they may also be advantageously selected from epicatechin and the dimers and trimers of epicatechin and catechin and the pharmaceutically acceptable analogs and derivatives of these compounds. It is believed that a mixture of two or more procyanidins such as the dimers and trimers of epicatechin and catechin and/or their pharmaceutically acceptable analogs and derivatives may be employed to therapeutic advantage, and in particular, a mixture of two or more proanthocyanidins such as epicatechin-4β→8-epicatechin, catechin-4α→8-epicatechin, epiafzelechin-4β→8-epicatechin, and epicatechin-4β→8-epicatechin-4β→8-epicatechin. It is believed that a mixture of substantially pure proanthocyanidins as a pharmaceutical composition is especially advantageous and has not been earlier suggested in the art.

Disclosed compositions contain one or more proanthocyanidins, each proanthocyanidin present in the composition in a proportion percentage or percentage purity that “significantly exceeds” a proportion percentage of the same proanthocyanidin's natural presence in a plant, or in an extract from the plant. For example, suppose that a particular proanthocyanidin is present in a plant in a percentage by weight of 0.01 percent, and is present in an extract of the plant in a percentage by weight of 1.0 percent. In a disclosed composition then, the same proanthocyanidin is present in the composition in a percentage by weight that is significantly greater than 0.01 percent or 1.0 percent, say 10 percent. Other proportionalities along this line may be applied as well, such as percentage composition or percentage presence by volume, or percent purity. By way of further example, without limiting the scope of invention to this or other examples, a PA is present in a tablet to be delivered orally in accordance with the disclosure herein. The PA is an isolated PA present in a percentage purity of 98.5% (that is, the PA is 98.5% pure, as measured by conventional purity indicia, such as for example the characteristic single sharp peak band on an HPLC). The particular PA is however only a 15% ingredient by weight in the tablet. The PA is known to be present in a fruit in a dry weight percentage of 0.06, while certain fruit extracts are known to contain up to 0.75 percent dry weight percent of the same PA. In this example, the PA is proportionally more present in the tablet than in the extract by a ratio of 20:1, and this is one measure of significantly exceeding the natural proportion percentage presence in a plant or extract of the plant.

In general, a PA present in a therapeutically administered dose form that has a percentage of the PA (by weight, dry weight, volume, or purity) that is 10 times (or more) greater than the natural percentage presence of the same PA in a plant is a percentage that “significantly exceeds” the natural percentage presence of the PA in the plant. When speaking of extracts of a plant in this context, it should be noted that only conventional or natural extracts are to be considered (juices, concentrates and the like, or extracts known and used for other purposes), not new extracts prepared after the priority date of this disclosure the effect of which is to concentrate the particular PA so as to negative a finding of “significantly exceeding”, as just defined. It should also be noted that in some cases, a finding of “significantly exceeding” may be justified with a ratio of as little as 2:1, but more preferably as great as 50:1 to 100:1.

In the example of a single PA compound with an excipient to make up the composition, it may be convenient merely to note and compare the percent purity of the compound in the composition, rather than its overall weight percentage, for purposes of the “significantly exceeding” standard, as claimed. In the case of mixtures of PA's it can be appropriate to view a combined percentage composition of the mixed PA's in the therapeutic dosage and compare that figure to a combined percentage presence of the same PA's in the natural plant or extract.

In any event, the purpose of the disclosed standard of measurement is to set forth a fair margin by which a claimed composition exceeds reading on the active ingredients' natural occurrence in plants and conventional extracts of plants.

Preferred compositions will contain proanthocyanidin that is at least substantially pure. Proanthocyanidin that is in substantially pure isolated or synthetic form may be advantageously employed as well. In general “pure” means better than 95% pure, and “substantially pure” PA means a PA purified by extraction or other known means or means disclosed herein such that the PA is present in the therapeutic dosage with only those impurities that can not readily nor reasonably be removed by the extraction or purification processes. “Isolated” means that the PA in question is not accompanied in the therapeutic form by significant quantities of other PA's. An “isolated pure” compound is a compound in isolated purified form such as is conventional for active ingredients in the pharmaceutical industry.

Methods of isolation of a proanthocyanidin from a plant material containing proanthocyanidins are disclosed. One method includes a) dissolving the plant material with methanol or the like non-polar solvent, b) loading the methanol-extracted plant material onto a silica gel column, c) eluting the column with a series of increasing proportions of methanol in chloroform to elute the proanthocyanidins, d) separating the proanthocyanidins in the extract by reverse phase HPLC, and e) collecting and freeze drying the separated and isolated proanthocyanidin, now deemed thereby to be “pure”. The series of methanol in chloroform elutions will beneficially include at least elutions of 10% methanol in chloroform, 20% methanol in chloroform, 40% methanol in chloroform, 50% methanol, and 100% methanol in chloroform. A preferred plant material is derived from Uncaria tomentosa.

A proanthocyanidin composition made from the disclosed isolation process is also disclosed. The composition eluted from the silica gel column with the 20% methanol in chloroform step of the series will contain primarily procyanidin dimers and trimers; the proanthocyanidin composition eluted from the silica gel column with the 40% methanol in chloroform step will contain primarily procyanidin trimers and tetramers; the composition eluted from the silica gel column with the 50% methanol in chloroform step will contain primarily procyanidin trimers, tetramers, pentamers, and hexamers; and the composition eluted from the silica gel column with the 100% methanol in chloroform step will contain primarily procyanidins tetramers, pentamers, hexamers, and oligomers of greater than six units.

A second isolation method includes a) dissolving the plant material with ethanol or the like non-polar solvent, b) loading the ethanol-extracted plant material onto a LH20 column, c) eluting the column with ethanol, followed by a series of increasing proportions of acetone in ethanol (and/or methanol) to elute the proanthocyanidins, d) separating the proanthocyanidins in the extract by reverse phase HPLC, and e) collecting and freeze drying the separated and isolated proanthocyanidin, now deemed thereby to be “pure”. The series of acetone in ethanol (and/or methanol) elutions will beneficially include at least elutions of 5% acetone in ethanol, 10% acetone in ethanol, 50% acetone in ethanol, 50% acetone in methanol and 100% methanol. A preferred plant material is derived from Uncaria tomentosa.

A proanthocyanidin composition made from the second disclosed isolation process is also disclosed. The composition eluted from the LH20 column with ethanol will contain primarily procyanidin dimers and trimers; the proanthocyanidin composition eluted from the LH20 column with the 5% acetone in ethanol step will contain primarily procyanidin dimers and trimers; the proanthocyanidin composition eluted from the LH20 column with the 10% acetone in ethanol step will contain primarily procyanidin dimers and trimers; the proanthocyanidin composition eluted from the LH20 column with the 50% acetone in ethanol step will contain primarily procyanidin dimers, trimers, and tetramers; the proanthocyanidin composition eluted from the LH20 column with the 50% acetone in methanol step will contain primarily procyanidin trimers, tetramers, pentamers, hexamers and oligomers of greater than six units; and the proanthocyanidin composition eluted from the LH20 column with the 100% methanol step will contain primarily procyanidin trimers, tetramers, pentamers, hexamers and oligomers of greater than six units.

A further method of treatment of an amyloid disease, or a disease characterized by α-synuclein or NAC fibrillogenesis, in a mammalian subject, is also disclosed. The method includes administering to the subject a therapeutically effective amount of the proanthocyanidin isolated by way of the disclosed isolation process.

In disclosed methods, the amyloid disease for treatment is selected from the group consisting of amyloid diseases associated with Alzheimer's disease, Down's syndrome, hereditary cerebral hemorrhage with amyloidosis of the Dutch type, inclusion body myositosis, the amyloidosis associated with type 2 diabetes, the amyloidosis associated with chronic inflammation, various forms of malignancy, and Familial Mediterranean Fever, the amyloidosis associated with multiple myeloma and other B-cell dyscrasias, the amyloidosis associated with the prion diseases including Creutzfeldt-Jakob disease, Gerstmann-Strausller syndrome, kuru, animal scrapie, and mad cow disease, the amyloidosis associated with long-term hemodialysis and carpal tunnel syndrome, the amyloidosis associated with endocrine tumors such as medullary carcinoma of the thyroid, and the α-synuclein disease is selected from the group consisting of Parkinson's disease, Lewy body disease and multiple system atrophy.

Another composition is disclosed as well. It includes a pharmaceutically acceptable carrier, diluent, or excipient, or the like, and a proanthocyanidin (PA), or proanthocyanidin compound, that may be found in the group of proanthocyanidin and proanthocyanidin compounds characterized by either Formula I or Formula II, or both (see FIGS. 54-56). The group also includes proanthocyanidins characterized by oligomeric combinations of Formula I and Formula II (see FIG. 56), and also includes any pharmaceutically acceptable salt of any of the foregoing proanthocyanidins. In the formula, n is an integer in the range of 2 to 20 and preferably 2-5 or even 2-3.

The PA is selectably present in the composition in an amount effective to treat an amyloid disease, or a disease characterized by α-synuclein or NAC fibrillogenesis, in a mammalian subject.

In the formulae shown, R₁ and R₂ are independently selected from hydrogen and hydroxy; R₃ is selected from the croup consisting of hydrogen, optionally substituted O-glycosyl, —C(O)-(optionally substituted aryl), and <(O)-(optionally substituted heteroaryl); and R₄ is selected from the group consisting of hydrogen, catechin, epicatechin, and gallates of catechin and epicatechin. The lines at the 2-, 3- and 4-position denote optional R and S (sometimes and alternatively referred to as a and B) stereochemical configurations. Generally the configuration at the 4-position is trans to the configuration at the 3-position. The lines at the 4- and 8-positions in Formula I and at the 4- and 6-positions in Formula II denote possible oligomer bonds between individual units as earlier discussed, and the each of the substitutions at R₁, R₂, R₃, and R₄, and each of the configurations at the 2-, 3-, and 4-positions, and each of the oligomer bond configurations of 4-8 and 4-6 are independently selected for each individual unit and may be different for unit in the oligomer series of units, though often the units of shorter oligomers are homogenous with one another.

The invention is described with reference to specific embodiments, plant species and parts, methods, procedures and the like. However, it will be recognized by those skilled in the art that various chemical substitutions can be made within the disclosed compounds without departing from the spirit and scope of the invention. In particular, it is known that polyphenols including flavanoids, procyanidins and proanthocyanidins can be isolated and/or purified from plant materials by a number of different methods. It will further be recognized that these alternate methods, and consequent changes in other steps of the method, such as use of different solvents or different columns for purification, and of procyanidins and proanthocyanidins from a composition of partially purified polyphenols, fall within the scope of the presently disclosed plant-derived extracts, and compounds derived thereof.

New methods for the treatment of the amyloid diseases are disclosed. The amyloid diseases include, but are not limited to, the amyloid associated with Alzheimer's disease, Down's syndrome, hereditary cerebral hemorrhage with amyloidosis of the Dutch type, inclusion body myositosis (wherein the specific amyloid is referred to as beta-amyloid protein or AB), the amyloid associated with chronic inflammation, various forms of malignancy and Familial Mediterranean Fever (wherein the specific amyloid is referred to as AA amyloid or inflammation-associated amyloidosis), the amyloid associated with multiple myeloma and other β-cell dyscrasias (wherein the specific amyloid is referred to as AL amyloid), the amyloid associated with type II diabetes (wherein the specific amyloid protein is referred to as amylin or islet amyloid polypeptide), the amyloid associated with the prion diseases including Creutzfeldt-Jakob disease, Gerstmann-Straussler syndrome, kuru and animal scrapie (wherein the specific amyloid is referred to as PrP amyloid), the amyloid associated with long-term hemodialysis and carpal tunnel syndrome (wherein the specific amyloid is referred to as beta.-microglobulin amyloid), the amyloid associated with senile cardiac amyloid and Familial Amyloidotic Polyneuropathy (wherein the specific amyloid is referred to as transthyretin or prealbumin), and the amyloid associated with endocrine tumors such as medullary carcinoma of the thyroid (wherein the specific amyloid is referred to as variants of procalcitonin). In addition, the α-synuclein protein which forms fibrils, and is Congo red and Thioflavin S positive, is found as part of Lewy bodies in the brains of patients with Parkinson's disease, Lewy body disease (Lewy in Handbuch der Neurologie, M. Lewandowski, ed., Springer, Berline pp. 920-933, 1912; Pollanen et al, J. Neuropath Exp Neurol. 52:183-191, 1993; Spillantini et al, Proc. Natl. Acad, Sci. USA 95:6469-673, 1998, Arai et al, Neurosc. Lett. 259:83-86, 1999), and multiple system atrophy. For purposes of this disclosure, Parkinson's disease, due to the fact that fibrils develop in the brains of patients with this disease (which are Congo red and Thioflavin S positive, and which contain predominant beta-pleated sheet secondary structure), should be regarded as a disease that also displays the characteristics of an amyloid-like disease.

Use of the inner bark and/or roots from Uncaria tomentosa (also referred to as Uña de Gato or Cat's claw) to isolate and use the amyloid inhibiting compounds for the treatment of amyloid formation, deposition, accumulation and/or persistence in Alzheimer's disease, type II diabetes other amyloidoses, and Parkinson's disease are disclosed. Uña de Gato or Cat's claw is also referred to as, but not limited to, Paraguayo, Garabato, Garbato casha, Tambor huasca, Una de gavilan, Hawk's claw, Nail of Cat, and Nail of Cat Schuler.

Use of extracts and/or compound derivatives thereof from plant matter related to the Rubiciaceae family, which includes but is not limited to the Uncaria genus, for the treatment of amyloid formation, deposition, accumulation and/or persistence in Alzheimer's disease, type II diabetes, other amyloidoses and Parkinson's disease are disclosed.

Use of extracts and/or compounds derived thereof from plant matter related to the various Uncaria species, which may include but not limited to, Uncaria tomentosa, Uncaria attenuata, Uncaria elliptica, Uncaria guianensis, Uncaria preropoda, Uncaria bernaysli, Uncaria ferra DC, Uncaria kawakamii, Uncaria rhyncophylla, Uncaria calophylla, Uncaria gambir, and Uncaria orientalis are also disclosed.

Use of commercially available pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, bark bundles and/or bark powder which contain Uncaria tomentosa and related plant materials for use to obtain extractable plant material, to treat patients with Alzheimer's disease, type II diabetes other amyloidoses, and Parkinson's disease are disclosed.

Use of polyphenols contained within Uncaria tomentosa and related plant materials for the treatment of amyloid formation, deposition, accumulation and/or persistence in Alzheimer's disease, type II diabetes, other amyloidoses and Parkinson's disease are disclosed.

It has been surprisingly discovered that proanthocyanidin type compounds purified from Uncaria tomentosa have anti-amyloid and anti-α-synuclein/NAC activity. Accordingly the present invention provides Uncaria tomentosa extracts and individual compounds derived thereof. The extract preferably comprises polyphenols(s), such as polyphenols of a least one proanthocyanidin selected from, but not limited to, epicatechin, catechin, epiafzelechin, procyanidin B2, procyanidin oligomers 2 though 10, preferably 2 through 5 or 4 through 10, procyanidin β4, procyanidin C1, and derivatives thereof.

Use of the proanthocyanidins contained within Uncaria tomentosa and related plant materials for the treatment of amyloid formation, deposition, accumulation and/or persistence in Alzheimer's disease, type II diabetes, other amyloidoses and Parkinson's disease are disclosed.

Use of the procyanidins contained within Uncaria tomentosa and related plant materials for the treatment of amyloid formation, deposition, accumulation and/or persistence in Alzheimer's disease, type II diabetes, other amyloidoses and Parkinson's disease are disclosed.

Use of epicatechin-4β→8-epicatechin, also known as procyanidin or proanthocyanidin B2, for the treatment of amyloid formation, deposition, accumulation and/or persistence in Alzheimer's disease, type II diabetes, other amyloidoses and Parkinson's disease is disclosed.

Use of catechin-4β→8-epicatechin, also known as procyanidin or proanthocyanidin B4, for the treatment of amyloid formation, deposition, accumulation and/or persistence in Alzheimer's disease, type I diabetes, other amyloidoses and Parkinson's disease is disclosed.

Use of epicatechin-4β→8-epicatechin-4β→8-epicatechin, also known as procyanidin or proanthocyanidin C1, for the treatment of amyloid formation, deposition, accumulation and/or persistence in Alzheimer's disease, type II diabetes, other amyloidoses and Parkinson's disease is disclosed.

Use of epiafzelechin-4β→8-epicatechin, for the treatment of amyloid formation, deposition, accumulation and/or persistence in Alzheimer's disease, type II diabetes, other amyloidoses and Parkinson's disease is also disclosed.

Methods to isolate the active amyloid inhibitory proanthocyanidins present within Uncaria tomentosa and related plant materials for use as potent agents which inhibit amyloid formation, amyloid deposition, amyloid accumulation, amyloid persistence, amyloid protein-amyloid protein interactions, and/or cause a dissolution/disruption of pre-formed or pre-deposited amyloid fibrils in Alzheimer's disease, type II diabetes, systemic AA amyloidosis, other amyloidoses and Parkinson's disease are also disclosed.

Compositions and methods involving administering to a subject a therapeutic dose of proanthocyanidins, epicatechin-4β→8-epicatechin, catechin-4α→8-epicatechin, epiafzelechin-4β→8-epicatechin, epicatechin-4β→8-epicatechin-4β→8-epicatechin or analogs or derivatives thereof (as disclosed herein) that inhibits amyloid deposition are disclosed. Accordingly, the compositions and methods of the invention are useful for inhibiting amyloidosis in disorders in which amyloid deposition occurs. The compounds of the invention can be used therapeutically to treat amyloidosis or can be used prophylactically in a subject susceptible to amyloidosis. The methods of the invention are based, at least in part, in directly inhibiting amyloid fibril formation, inhibiting amyloid fibril growth, and/or causing dissolution/disruption of preformed amyloid fibrils.

Pharmaceutical compositions for treating amyloidosis are disclosed. The pharmaceutical compositions include a therapeutic compound of the invention in an amount effective to inhibit amyloid deposition and a pharmaceutically acceptable vehicle.

The proanthocyanidin composition of the invention which can be administered as a pharmaceutical composition is disclosed. The pharmaceutical composition, may include, but is not limited to, a proanthocyanidin extract or purified compound, and a pharmaceutically acceptable carrier, such as lactose, cellulose, or equivalent, or contained within a pharmaceutical dosage, such as a capsule or tablet.

Use of any and all synthetic compounds made similar to procyanidins, proanthocyanidins, epicatechin-4β→8-epicatechin (i.e. procyanidin B2), catechin-4α→8-epicatechin (i.e. procyanidin B4), epicatechin-4β→8-epicatechin-4β→8-epicatechin (i.e. procyanidin C1), epiafzelechin-4β→8-epicatechin, or analogs or derivatives thereof, including proanthocyanidins B1, B2, B3, B4, B5, B6, B7, B8, C1 or C2, for use as potent agents which inhibit amyloid formation, amyloid deposition, amyloid accumulation, amyloid persistence, amyloid protein-amyloid protein interactions, and/or cause a dissolution/disruption of pre-formed or pre-deposited amyloid fibrils in Alzheimer's disease, type II diabetes, systemic AA amyloidosis, other amyloidoses and Parkinson's disease is disclosed.

Preventing or treating amyloidosis in a mammal by administering a proanthocyanidin composition, which may include but not limited to, a proanthocyanidin extract, a proanthocyanidin compound, a proanthocyanidin polymer or mixture thereof, to the mammal in an amount and for a time sufficient to prevent, reduce, or eliminate amyloid formation, deposition, accumulation and/or persistence, and thereby lead to effective treatments for Alzheimer's disease, Parkinson's disease, type 2 diabetes, systemic AA amyloidosis, and other amyloid disorders is disclosed.

A method of isolation to purify and identify the procyanidins, proanthocyanidins, epicatechin-4β→8-epicatechin (i.e. procyanidin B2), catechin-4α→8-epicatechin (i.e. procyanidin B4), epicatechin-4β→8-epicatechin-4β→8-epicatechin (i.e. procyanidin C1) and epiafzelechin-4β→8-epicatechin, or analogs or derivatives thereof from Uncaria tomentosa are disclosed. In one such method, an extract prepared to produce commercially obtained pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, bark bundles and/or bark powder, using the methods described in the present invention.

Methods of extraction as described herein to provide purified compounds from Uncaria tomentosa and related plant materials for promoting mental alertness and for inhibiting the formation of brain amyloid deposits in a subject are disclosed.

Purified compounds from Uncaria tomentosa and related plant materials for mental acuity; to promote mental alertness; to provide nutritional support for age or related cognitive or memory decline; to promote cognitive well being; to support brain function; to improve cognitive ability, mental performance or memory; to promote concentration and mental sharpness; to improve mental vitality; to promote greater mental clarity and alertness; to improve short term memory, for age associated cognitive or memory decline; to support normal brain function; to enhance learning or memory; to improve concentration; to enhance mental performance; to reduce mental decline; to reduce likelihood of age related brain disorders; to maintain good brain health; to reduce, eliminate, prevent, inhibit or disrupt/dissolve amyloid fibril or protein deposits, brain associated amyloid fibril deposits or brain associated amyloid protein deposits, amyloid fibril formation and growth or age associated amyloid fibril formation and growth, brain associated amyloid fibril formation and growth; to support healthy pancreatic function; to promote pancreatic function by helping to promote normal insulin function; to reduce, eliminate, prevent, inhibit or disrupt/dissolve amyloid fibril or protein deposits, and pancreas associated amyloid fibril formation and growth, are also disclosed.

### Amyloid and Amyloidosis

Amyloid is a generic term referring to a group of diverse but specific extracellular protein deposits which all have common morphological properties, staining characteristics, and X-ray diffraction spectra. Regardless of the nature of the amyloid protein deposited all amyloids have the following characteristics: 1) showing an amorphous appearance at the light microscopic level, appearing eosinophilic using hematoxylin and eosin stains; 2) staining with Congo red and demonstrating a red/green birefringence as viewed under polarized light (Puchtler et al., J. Histochem. Cytochem. 10:355-364, 1962), 3) containing a predominant beta-pleated sheet secondary structure, and 4) ultrastructurally consisting of non-branching fibrils of indefinite length and with a diameter of 7-10 nm. Amyloidoses and “amyloid diseases” today are classified according to the specific amyloid protein deposited. The amyloids include, but are not limited to, the amyloid associated with Alzheimer's disease, Down's syndrome, hereditary cerebral hemorrhage with amyloidosis of the Dutch type and inclusion body myositosis (where the specific amyloid is referred to as beta-amyloid protein or AB), the amyloid associated with chronic inflammation, various forms of malignancy and familial Mediterranean fever (where the specific amyloid is referred to as AA amyloid or inflammation-associated amyloid), the amyloid associated with multiple myeloma and other β-cell dyscrasias (where the specific amyloid is referred to as AL amyloid), the amyloid associated with type II diabetes (where the specific amyloid is referred to as amylin or islet amyloid), the amyloid associated with the prion diseases including Creutzfeldt-Jakob disease, Gerstmann-Straussler syndrome, kuru, and scrapie (where the specific amyloid is referred to as PrP amyloid), the amyloid associated with long-term hemodialysis and carpal tunnel syndrome (where the specific amyloid is referred to as beta₂-microglobulin amyloid), the amyloid associated with senile cardiac amyloid and familial amyloidotic polyneuropathy (where the specific amyloid is referred to as prealbumin or transthyretin amyloid), and the amyloid associated with endocrine tumors such as medullary carcinoma of the thyroid (where the specific amyloid is referred to as variants of procalcitonin).

Although amyloid deposits in clinical conditions share common physical properties relating to the presence of a beta-pleated sheet conformation, it is now clear that many different chemical types exist and additional ones are likely to be described in the future. It is currently thought that there are several common pathogenetic mechanisms that may be operating in amyloidosis in general. In many cases, a circulating precursor protein may result from overproduction of either intact or aberrant molecules (for example, in plasma cell dyscrasias), reduced degradation or excretion (serum amyloid A in some secondary amyloid syndromes and beta₂-microglobulin in long-term hemodialysis), or genetic abnormalities associated with variant proteins (for example, familial amyloidotic polyneuropathy). Proteolysis of a larger protein precursor molecule occurs in many types of amyloidosis, resulting in the production of lower molecular weight fragments that polymerize and assume a beta-pleated sheet conformation as tissue deposits, usually in an extracellular location. The precise mechanisms involved and the aberrant causes leading to changes in proteolytic processing and/or translational modification are not known in most amyloids.

Systemic amyloid diseases which include the amyloid associated with chronic inflammation, various forms of malignancy and familial Mediterranean fever (i.e. AA amyloid or inflammation-associated amyloidosis) (Benson and Cohen, Arth. Rheum. 22:3642, 1979; Kamei et al, Acta Path. Jpn. 32:123-133, 1982; McAdam et al., Lancet 2:572-573, 1975; Metaxas, Kidney Int. 20:676-685, 1981), and the amyloid associated with multiple myeloma and other β-cell dyscrasias (i.e. AL amyloid) (Harada et al., J. Histochem. Cytochem. 19:1-15, 1971), as examples, are known to involve amyloid deposition in a variety of different organs and tissues generally lying outside the central nervous system. Amyloid deposition in these diseases may occur, for example, in liver, heart, spleen, gastrointestinal tract, kidney, skin, and/or lungs (Johnson et al, N. Engl. J. Med. 321:513-518, 1989). For most of these amyloidoses, there is no apparent cure or effective treatment and the consequences of amyloid deposition can be detrimental to the patient. For example, amyloid deposition in the kidney may lead to renal failure, whereas amyloid deposition in the heart may lead to heart failure. For these patients, amyloid accumulation in systemic organs leads to eventual death generally within 3-5 years. Other amyloidoses may affect a single organ or tissue such as observed with the AB amyloid deposits found in the brains of patients with Alzheimer's disease and Down's syndrome: the PrP amyloid deposits found in the brains of patients with Creutzfeldt-Jakob disease, Gerstmann-Straussler syndrome, and kuru; the islet amyloid (amylin) deposits found in the islets of Langerhans in the pancreas of 90% of patients with type II diabetes (Johnson et al, N. Engl. J. Med. 321:513-518, 1989; Lab. Invest 66:522 535, 1992); the beta₂-microglobulin amyloid deposits in the medial nerve leading to carpal tunnel syndrome as observed in patients undergoing long-term hemodialysis (Geyjo et al., Biochem. Biophys. Res. Comm. 129:701-706, 1985; Kidney Int. 30:385-390, 1986); the prealbumin/transthyretin amyloid observed in the hearts of patients with senile cardiac amyloid; and the prealbumin/transthyretin amyloid observed in peripheral nerves of patients who have familial amyloidotic polyneuropathy (Skinner and Cohen, Biochem. Biophys. Res. Comm. 99:1326-1332, 1981; Saraiva et al, J. Lab. Clin. Med. 102:590-603, 1983; J. Clin. Invest 74:1119, 1984; Tawara et al, J. Lab. Clin. Med. 98:811-822, 1989).

### Alzheimer's Disease and the Aging Population

Alzheimer's disease is a leading cause of dementia in the elderly, affecting 5-10% of the population over the age of 65 years (A Guide to Understanding Alzheimer's Disease and Related Disorders, Jorm, ed., New York University Press, New York, 1987). In Alzheimer's disease, the parts of the brain essential for cognitive processes such as memory, attention, language, and reasoning degenerate, robbing victims of much that makes us human, including independence. In some inherited forms of Alzheimer's disease, onset is in middle age, but more commonly, symptoms appear from the mid-60's onward. Alzheimer's disease today affects 4-5 million Americans, with slightly more than half of these people receiving care at home, while the others are in many different health care institutions. The prevalence of Alzheimer's disease and other dementias doubles every 5 years beyond the age of 65, and recent studies indicate that nearly 50% of all people age 85 and older have symptoms of Alzheimer's disease (2000 Progress Report on Alzeimer's Disease, National Institute on Agingational Institute of Health). 13% (33 million people) of the total population of the United States are age 65 and older, and this percentage will climb to 20% by the year 2025 (2000 Progress Report on Alzheimer's Disease).

Alzheimer's disease also puts a heavy economic burden on society. A recent study estimated that the cost of caring for one Alzheimer's disease patient with severe cognitive impairments at home or in a nursing home, is more than $47,000 per year (A Guide to Understanding Alzheimer's Disease and Related Disorders). For a disease that can span from 2 to 20 years, the overall cost of Alzheimer's disease to families and to society is staggering. The annual economic toll of Alzheimer's disease in the United States in terms of health care expenses and lost wages of both patients and their caregivers is estimated at $80 to $100 billion (2000 Progress Report on Alzeimer's Disease).

Tacrine hydrochloride (“Cognex”), the first FDA approved drug for Alzheimer's disease, is a acetylcholinesterase inhibitor (Cutler and Sramek, N. Engl., Med. 328:808 810, 1993). However, this drug has showed limited success in producing cognitive improvement in Alzheimer's disease patients and initially had major side effects such as liver toxicity. The second more recently FDA approved drug, donepezil (“Aricept”), which is also an acetylcholinesterase inhibitor, is more effective than tacrine, by demonstrating slight cognitive improvement in Alzheimer's disease patients (Barner and Gray, Ann. Pharmacotherapy 32:70-77, 1998; Rogers and Friedhoff, Eur. Neuropsych. 8:67-75, 1998), but is not believed to be a cure. Therefore, it is clear that there is a need for more effective treatments for Alzheimer's disease patients.

### Amyloid as a Therapeutic Target for Alzheimer's Disease

Alzheimer's disease is characterized by the deposition and accumulation of a 39-43 amino acid peptide termed the beta-amyloid protein, Aβ or β/A4 (Glenner and Wong, Biochem. Biophys. Res. Comm. 120:885-890, 1984; Masters et al., Proc. Natl. Acad. Sci. USA 82:4245-4249, 1985; Husby et al., Bull. WHO 71:105-108, 1993). AB is derived by protease cleavage from larger precursor proteins termed beta-amyloid precursor proteins (or BPPs) of which there are several alternatively spliced variants. The most abundant forms of the BPPs include proteins consisting of 695, 751 and 770 amino acids (Tanzi et al., Nature 331:528-530, 1988; Kitaguchi et al., Nature 331:530-532, 1988; Ponte et al., Nature 331:525-527, 1988).

The small Aβ peptide is a major component which makes up the amyloid deposits of “plaques” in the brains of patients with Alzheimer's disease. In addition, Alzheimer's disease is characterized by the presence of numerous neurofibrillary “tangles”, consisting of paired helical filaments which abnormally accumulate in the neuronal cytoplasm (Grundke-Iqbal et al., Proc. Nat. Sci. USA 83:4913-4917, 1986; Kosik et al., Proc. Natl. Acad. Sci. USA 83:404-44048, 1986; Lee et al., Science 251:675-678, 1991). The pathological hallmark of Alzheimer's disease is therefore the presence of “plaques” and “tangles”, with amyloid being deposited in the central core of the plaques. The other major type of lesion found in the Alzheimer's disease brain is the accumulation of amyloid in the walls of blood vessels, both within the brain parenchyma and in the walls of meningeal vessels that lie outside the brain. The amyloid deposits localized to the walls of blood vessels are referred to as cerebrovascular amyloid or congophilic angiopathy (Mandybur, J. Neuropath. Exp. Neurol. 45:79-90, 1986; Pardridge et al., J. Neurochem. 49:1394-1401, 1987).

For many years there has been an ongoing scientific debate as to the importance of “amyloid” in Alzheimer's disease, and whether the “plaques” and “tangles” characteristic of this disease were a cause or merely a consequence of the disease. Within the last few years, studies now indicate that amyloid is indeed a causative factor for Alzheimer's disease and should not be regarded as merely an innocent bystander. The Alzheimer's Aβ protein in cell culture has been shown to cause degeneration of nerve cells within short periods of time (Pike et al., Br. Res. 563:311-314, 1991; J. Neurochem. 64:253-265, 1995). Studies suggest that it is the fibrillar structure (consisting of a predominant beta-pleated sheet secondary structure), characteristic of all amyloids, that is responsible for the neurotoxic effects. Aβ has also been found to be neurotoxic in slice cultures of hippocampus (Harrigan et al., Neurobiol. Aging 16:779-789, 1995) and induces nerve cell death in transgenic mice (Games et al., Nature 373:523-527, 1995; Hsiao et al., Science 274:99-102, 1996). Injection of the Alzheimer's Aβ into rat brain also causes memory impairment and neuronal dysfunction Flood et al., Proc. Natl. Acad. Sci. USA 88:3363-3366, 1991; Br. Res. 663:271-276, 1994).

Probably, the most convincing evidence that Aβ amyloid is directly involved in the pathogenesis of Alzheimer's disease comes from genetic studies. It has been discovered that the production of Aβ can result from mutations in the gene encoding, its precursor, beta amyloid precursor protein (Van Broeckhoven et al., Science 248:1120-1122, 1990; Murrell et al., Science 254:97-99, 1991; Haass et al., Nature Med 1:1291-1296, 1995). The identification of mutations in the beta-amyloid precursor protein gene which causes early onset familial Alzheimer's disease is the strongest argument that amyloid is central to the pathogenetic process underlying this disease. Four reported disease-causing mutations have now been discovered which demonstrate the importance of Aβ in causing familial Alzheimer's disease (reviewed in Hardy, Nature Genet. 1:233-234, 1992). All of these studies suggest that providing a drug to reduce, eliminate or prevent fibrillar Aβ formation, deposition, accumulation and/or persistence in the brains of human patients will serve as an effective therapeutic.

Discovery and identification of new compounds or agents as potential therapeutic agents to arrest amyloid deposition, accumulation and/or persistence that occur in Alzheimer's disease and other amyloidoses are desperately sought,

### Parkinson's Disease and α-Synuclein Fibril Formation

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosc. Lett. 259:83-86, 1999), a 140-amino acid protein (Ueda et al., Proc. Natl. Acad. Sci. U.S.A. 90:11282-11286, 1993). Two dominant mutations in α-synuclein causing familial early onset Parkinson's disease have been described suggesting that Lewy bodies contribute mechanistically to the degeneration of neurons in Parkinson's disease (Polymeropoulos et al., Science 276:2045-2047, 1997; Kruger et al., Nature Genet. 18: 106-108, 1998). Recently, in vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999). Most importantly, both Parkinson's disease-linked α-synuclein mutations accelerate this aggregation process that suggests that such in vitro studies may have relevance for Parkinson's disease pathogenesis. α-Synuclein aggregation and fibril formation fulfills of the criteria of a nucleation-dependent polymerization process (Wood et al., J. Biol. Chem. 274:19509-19512, 1999). In this regard α-synuclein fibril formation resembles that of Alzheimer's beta-amyloid protein (Aβ) fibrils. α-Synuclein recombinant protein, and non-amyloid component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Pro. Natl. Acad. Sci. U.S.A 90:11282-11286, 1993).

In addition, accumulation of α-synuclein/NAC is also a cytopathological feature common to Lewy body disease and multiple system atrophy (Wakabayashi et al, Acta Neuropath. 96:445-452, 1998; Piao et al, Acta Neuropath. 101:285-293, 2001). Multiple system atrophy is a sporadic neurodegenerative disease in adults characterized by neuronal and glial cytoplasmic inclusions, containing α-synuclein/NAC.

Parkinson's disease α-synuclein/NAC fibrils, like the Aβ fibrils of Alzheimer's disease, also consist of a predominant beta-pleated sheet structure. It is therefore believed that compounds found to inhibit Alzheimer's disease Aβ amyloid fibril formation can also be anticipated to be effective in the inhibition of α-synuclein and/or NAC fibril formation. These compounds would therefore also serve as therapeutics for Parkinson's disease, in addition to having efficacy as a therapeutic for Alzheimer's disease and other amyloid disorders.

### Islet Amyloid Polypeptide (IAPP) and Type 2 Diabetes

Islet amyloid deposits are observed in ˜90% of patients with well-established type 2 diabetes and would appear to be a characteristic feature of the disease process (Westermark, J. Med. Sci. 77:91-94, 1972; Clark et al, Diabetes Res. 9:151-159, 1988). In many patients the deposits are widespread and affect many islets. The degree of islet (predominantly β-cell) mass that has been replaced by amyloid may be a marker for the severity of the diabetic disease process, with those individuals requiring insulin treatment having the greatest islet mass reduction and amyloid formation (Westermark, Amyloid: Int. J. Exp. Clin. Invest. 1:47-60, 1994). Since islet amyloid has been observed in autopsy samples obtained from different populations, it appears to be a phenomenon common to the disease rather than to a subpopulation of individuals with the syndrome (Westermark, J. Med. Sci. 77:91-94, 1972; Clark et al., Diabetes Res. 9:151-159, 1988). The prevalence of islet amyloid deposits increases with age (Bell, Am. J. Path. 35:801-805, 1959), which is not surprising because normal aging is associated with a deterioration in glucose tolerance and an increased prevalence of type 2 diabetes (Davidson, Metabolism 28:687-705, 1979).

The major protein in islet amyloid is a 37-amino acid peptide known as islet amyloid polypeptide (IAPP) or amylin. IAPP is a known normal secretory product of the pancreatic β-cells (Kanh et al, Diabetes 39:634-638, 1990) that is stored in insulin-bearing cytoplasmic granules (Clark et al, Cell Tissue Res. 257:179-185, 1989). It has long been questioned whether the deposition of islet amyloid is involved in or merely a consequence of the pathogenesis of type 2 diabetes. However, a number of studies now suggest that in fact islet amyloid formation, deposition and persistence may be an important primary factor leading to β-cell dysfunction and cell death, hyperglycemia, and in the development of type 2 diabetes.

IAPP has been hypothesized to have an important role in the pathogenesis of type 2 diabetes through its impairment of β-cell function and reduction of β-cell mass (Johnson et al, N. Engl. J. Med. 321:513-518, 1989). Besides being able to form islet amyloid deposits that replace β-cell mass, amyloid fibrils appear to damage islets directly. Studies in which islets were incubated in the presence of human or rat IAPP demonstrated that human IAPP formed amyloid fibrils in a concentration-dependent manner and was associated with the death of pancreatic islet β-cells (Lorenzo et al, Nature 368:756-760, 1994). Cell death did not occur in the presence of rat IAPP that does not form amyloid fibrils (Lorenzo et al, Nature 368:756-760, 1994).

Studies involving transgenic mouse models have allowed further insight into the role of islet amyloid in the pathogenesis of type 2 diabetes. More recent studies do suggest that development of TAPP-derived islet amyloid does not depend on hyperglycemia and is progressive (Verchere et al, Proc. Acad. Sci. U.S.A. 93:3492-3496, 1996). In these latter studies hyperglycemia developed in only 31% of male transgenic mice, and in 14% of male nontransgenic animals. When pancreatic sections from these mice were examined, islet amyloid was found in every transgenic mouse with diabetes. However, two-thirds of male transgenic animals that were normoglycemic also developed islet amyloid deposits indicating that hyperglycemia was not a prerequisite for islet amyloid formation. The data from these and other studies further suggested that human IAPP fibrils may be cytotoxic to dwells and thus could produce early alterations in islet function (Lorenzo et al, Nature 368:756-760, 1994; Janson et al, Diabetes 47:A250, 1998). Islet amyloid deposition appears to be an early feature of the islet lesion of type 2 diabetes and progressive accumulation of islet amyloid is associated with further β-cell mass reduction (Clark et al, Diabetes Res. 9:151-159, 1988; Westermark and Wilander, Diabetologia 15:417-421, 1978). Thus, a progressive reduction in islet mass caused by increased amyloid deposition is associated with a progressive impairment in insulin secretion, reduction in glucose tolerance, and eventually the development of fasting hyperglycemia. The studies in transgenic animals suggest not just that hyperglycemia is associated with the development of islet amyloid, but that amyloid contributes to the development of hyperglycemia by replacing β-cells. These studies as a whole suggest that islet amyloid formation plays a central role in the development of β-cell failure of type 2 diabetes. Therefore, agents or compounds able to inhibit or disrupt islet amyloid (i.e. IAPP or amylin) formation, deposition, accumulation or persistence may lead to new potential treatments for type 2 diabetes.

Uncaria tomentosa

The herb Uncaria tomentosa, also known as “Uña de Gato” (in Spanish) or “Cat's claw” (in English) refers to a woody vine that grows within the Peruvian Amazon rain forest. This slow growing vine takes 20 years to reach maturity, and can grow over 100 feet in length as it attaches and wraps itself around the native trees. It is found abundantly in the foothills, at elevations of two to eight thousand feet. The vine is referred to as “Cat's claw” because of its distinctive curved claw-like thorns that project from the base of its leaves. The native Indian tribes traditionally have boiled the inner bark and root of the herb to make a tea decoction and regard Uncaria tomentosa as a sacred medicinal plant. The highly effective properties contained within the inner bark of this plant are believed to have a profound and positive influence on the body, although scientific medical data is generally lacking on its potential benefits in humans. The alkaloids and phytochemicals in the inner bark of Uncaria tomentosa are almost identical to those found in the root, and harvesting this way preserves the plant and provides for the future of the rainforest.

Some of the active substances present in Uncaria tomentosa are alkaloids, which occur in the plant and its watery extract as a complex bound to tannins. In this form, only little of them can be activated. The complexes get split by the acid milieu of the stomach; the alkaloids get transformed into their hydrochloride form, and in this way, get well absorbed. A darker Uncaria tomentosa extract means more tannin is present and beneficial alkaloids are locked up with the tannins, which have formed a non-bioavailable and poorly absorbed complex A light golden color of Uncaria tomentosa suggests that there are less tannins, and more alkaloids available in the extract.

Uncaria tomentosa is one of the most important plants in the South American Peruvian rainforest. A number of oxindole alkaloids have already been isolated from the inner bark of this plant. Two US patents (U.S. Pat. No. 4,844,901 and U.S. Pat. No. 4,940,725) describe the isolation and use of six oxindole alkaloids from Uncaria tomentosa, which are believed to be “suitable for the unspecified stimulation of the immunologic system”. These oxindole alkaloids are believed to provide a general boost to the immune system as well as have a profound effect on the ability of white blood cells and macrophages to phagocytize harmful microorganisms and foreign matter. The most immunologically active alkaloid appears to be alloisopteropodine, isomer A, a pentacyclic oxindole alkaloid (U.S. Pat. No. 4,940,725).

Although some health care providers have suggested that Uncaria tomentosa may be used to treat a variety of ailments, nowhere has there been any use or suggestion of use, of this plant or extracts thereof, or compounds derived thereof, for the treatment of amyloid formation, deposition, accumulation and/or persistence, such as that which occurs in the amyloidoses, including Alzheimer's disease and Parkinson's disease. The present invention clearly demonstrates the effectiveness of Uncaria tomentosa derived compounds, including procyanidins and proanthocyanidins, for the treatment of amyloidosis associated with Alzheimer's disease, type 2 diabetes, systemic AA amyloidosis, and other amyloid diseases, as well as for the treatment of α-synuclein fibril formation and accumulation, such as that observed in patients with Parkinson's disease.

### Proanthocyanidins, Procyanidins, Flavanoids and Tannins

Proanthocyanidins are polyphenolic molecules occurring naturally in fruits, berries and other plant material. These molecules belong to the flavanoid family of compounds. The flavanoid polyphenolics include the catechins, anthocyanins, and proanthocyanidins. Proanthocyanidins are also known in the art as condensed tannins, leucoanthocyanidins, leucodelphinins, leucocyanins, anthocyanogens, epicatechin-catechin polymers or procyanidins. For a review of procyanidins and proanthocyanidins, see Santos-Buelga and Scalbert, J. Sc. Food Agri. 80-1094-1117, 2000, which is incorporated herein by reference as if fully set forth, and is discussed in detail below.

Proanthocyanidin oligomers or polymers useful for the present anti-amyloid activity are comprised of monomeric units of leucoanthocyanidins. Leucoanthocyanidins are generally monomeric flavanoids which include catechins, epicatechins, gallocatechins, galloepicatechins, flavanols, flavonols, and flavan-3,4-diols, leucocyanidins and anthocyanidins. The therapeutically effective proanthocyanidin polymers have from 2 to 20 flavanoid units, and more preferably from 2 to 11 flavanoid units.

Proanthocyanidins polymers or oligomers are known to have varying numbers of flavanoid units, and have been reported for example in Mattice et al, Phytochem. 23:1309-1311, 1984; Czochanska et all J.C.S. Chem. Comm. 375, 1979; Jones et al, Photochemistry, 15:1407-1409, 1976. Proanthocyanidin oligomers having the recited ranges of flavanoid units and described in these references are incorporated herein by reference as if their disclosure was fully set forth herein.

Procyanidins, also referred to as proanthocyanidins, are polymeric or oligomeric compounds composed of epicatechin and catechin residues. Disclosed compounds include dimers of epicatechin and catechin residues, and trimers of epicatechin. Catechin and epicatechin residues may be combined in all possible combinations in polymeric procyanidins up to molecular weights of up to about 10,000 daltons. Proanthocyanidin polymers are known to have a varying number of flavanoid units. The polymers preferably contain two to fifteen monomeric flavanoid subunits, most preferably two to ten subunits.

Tannins are classically divided into 2 groups. Hydrolysable tannins are esters of phenolic acids and a polyol, usually glucose. The phenolic acids are either gallic acid in gallotannins or other phenolic acids derived from the oxidation of galloyl residues in ellagitannins. Proanthocyanidins, forming the second group of tannins, are far more common in our diet. They are polymers made of elementary flavan-3-ol units A key feature of proanthocyanidins is that they yield anthocyanidins upon heating in acidic media, hence their name (reviewed in Santos-Buelga and Scalbert, J. Sc. Food Agri. 80: 1094-1117, 2000). Structurally, tannins possess 12-16 phenolic groups and 5-7 aromatic rings per 1000 units of relative molecular mass (E. Haslam, Practical Polyphenoics—from Structure to Molecular Recognition and Physiological Action, Cambridge University Press, Cambridge, 1998). This feature, together with their high molecular weight, clearly makes the tannins and similar phenolic polymers found in processed products such as red wine or black tea different both in structure and properties from the low-molecular-weight phenolic acids and monomeric flavanoids. The phenolic polymers, formed by enzymatic and/or chemical transformation of simple flavanols, proanthocyanidins and other phenolic compounds, are called tannin-like compounds.

Proanthocyanidins are polymeric flavan-3-ols whose elementary units are linked by C—C and occasionally C—O—C bonds. The flavan-3-ol units have the typical C6-C3-C6 flavanoid skeleton. The three rings are distinguished by the letters A, B and C (see FIG. 1). They differ structurally according to the number of hydroxyl groups on both aromatic rings and the stereochemistry of the asymmetric carbons on the heterocycle. The most common proanthocyanidins in food are procyanidins with a 3′,4′-dihydroxy substitution on the B ring and prodelphinidins with a 3′,4′,5′-trihydroxy substitution. Procyanidins or mixed procyanidins/prodelphinidins are most common in food. Propelargonidins with 4′-hydroxy B-rings are relatively rare in food sources, bit notably disclosed herein in the form of epiafzelechin. The three carbons C2, C3, C4 of the flavanol heterocycle are asymmetric and may occur in different configurations. With some very rare exceptions, the configuration of C2 is R. Flavan-3-ol units with the 2S configuration are distinguished by the prefix enantio(ent-), The stereochemistry of the C2-C3 linkage may be either trans (2R,3S) or cis (2R,3R) as in (+)-(gallocatechin and (−)-epi(gallocatechin polymers respectively The interflavan bond at C4 is always trans with respect to the hydroxy group at C3 (E. Haslam, Practical Polyphenoics—from Structure to Molecular Recognition and Physiological Action, Cambridge University Press, Cambridge, 1998).

The most usual interflavanol linkages are C—C bonds established between the C4 of one flavanoid unit (“extension or upper unit”). Such proanthocyanidins belong to the so-called B-type (dimeric) and C type (trimeric) proanthocyanidins. Compounds with doubly linked units (one C—C and one C—O; “A type linkage”) have also been reported in some food sources such as tea leaf, cocoa and cranberry fruits (L J. Porter, Flavans and proanthocyanidins, in The Flavanoids—Advances in Research Since 186, Ed. by J B Harborne, Chapman and Hall, London, pp. 23-55, 1994). In these A-type proanthocyanidins an additional ether linkage between the C2 of the upper unit and the oxygen-bearing C7 or C5 of the lower one is formed in addition to the usual C4-C8 or C4-C6 bond.

Initially oligomeric proanthocyanidins were named by an alpha-numeric system, with a letter A, B or C to describe the type of interflavanol linkage; a number was added to the letter as they were detected (Thompson et al, J. Chem. Soc. Perkins Trans. 1: 1387-1399, 1972). A new nomenclature was later introduced to name an increasing number of new structures. It is based on that utilized for the polysaccharides (Hemingway et al, J. Chem. Soc. Perkins Trans. 1:1387-1399, 1972). In this nomenclature, the elementary units of the oligomers are designated with the name of the corresponding flavan-3-ol monomers. The interflavanol linkage and its direction are indicated with an arrow (4→) and its configuration at C4 is described as ∝ or β. In type-A doubly linked proanthocyanidins, both linkages are indicated. It is unnecessary to indicate to indicate oxygen in the additional ether bond since it is obvious from the substitution pattern of catechin lower units (U Porter, in The Flavanoids—Advances in Research since 1980, Ed. by J B Harborne, Chapman and Hall, London, pp. 21-62, 1988). For instance, according to this nomenclature, procyanidin dimer B1 becomes epicatechin-4β→8-catechin and dimer A2 becomes epicatechin-2β→7, 4β→8-epicatechin.

Flavanol units can bear various acyl or glycosyl substituents. The most common acyl substituent is gallic acid which forms an ester with the hydroxyl n the C3 position, as in tea (Nonaka et al, Chem. Pharmaceutic. Bull. 31:3906-3914, 1983) and wine (Prieur et al, Phyochem, 36:781-784, 1994). Several glycosylated proanthocyanidin oligomers have also been characterized. The sugar is generally linked to the hydroxyl group at the C3 position (Ishimaru et al, Phytochemistry 26:1167-1170, 1987; Zhang et al, Phytochemistry 27:3277-3280, 1988), but also at the C5 position (Gujer et al, Phytochemistry 25:1431-136, 1986). Although proanthocyanidins heterosides are less frequently reported than other flavanoid glycosides, their occurrence may be underestimated, as sugars are frequently associated with purified proanthocyanidin polymers (Porter et al, Phytohemistry 24:567-569, 1985; Mathews et al, J. Agric. Food Chem. 45:1195-1201, 1997). Such variations, and other variations disclosed herein, are included with the scope of disclosure of the disclosed proanthocyanidins.

More recently, the introduction of electrospray mass spectrometry techniques coupled to liquid chromatography led to a more detailed characterization of proanthocyanidin polymers. Such methods were employed in the present invention to identify procyanidins and proanthocyanidins derived from Uncaria tomentosa which demonstrate potent anti-amyloid and anti-α-synuclein/NAC activity.

### BRIEF DESCRIPTION OF THE DRAWINGS

FIG. 1 is an illustration demonstrating the basic structure of proanthocyanidins, including proelargonidins (where R₁, R₂═H); procyanidins (where R₁═H, R₂═OH); and prodelphinins (where R₁, R₁═OH).

FIG. 2 is a HPLC tracing using method 1 (see Example 2, Table 1 for details) demonstrating the separation of PTI-777. Using this method, there is a good separation of H1 and H2 peaks.

FIG. 3 is a HPLC tracing using method 2 (see Example 2, Table 1 for details) demonstrating the separation of H1 and H2 from PTI-777 following silica gel chromatography and elution with 20% methanol in chloroform.

FIG. 4 is a HPLC tracing using method 1 (see Example 2, Table 1 for details) demonstrating the separation of mostly H1 (with less H2) after fractioning PTI-777 using silica gel chromatography, followed by HPLC.

FIG. 5 is a HPLC tracing using method 1 (see Example 2, Table 1 for details) demonstrating the isolation of pure H2 from PTI-777, after fractionating PTI-777 using silica gel chromatography, followed by HPLC.

FIG. 6 is a ¹H NMR spectrum of peak H2 derived from PTI-777.

FIG. 7 is a ¹³C NMR spectrum of peak H2 derived from PTI-777.

FIG. 8 is a ¹³C NMR spectrum of peak H2 in deteroacetone (instead of deuteromethanol).

FIG. 9 is a ¹H NMR spectrum of peak H2 in deteroacetone (instead of deuteromethanol).

FIG. 10 is the peracetate structure of a sample of pure H2 following acetylation.

FIG. 11 is the ¹H NMR spectrum of the H2 peracetate in CDCl₃.

FIG. 12 is the ¹³C NMR spectrum of the H2 peracetate in CDCl₃.

FIG. 13 is the CIGAR ¹H-¹³C correlation spectrum (low resolution) of the H2 peracetate.

FIG. 14 is the CIGAR ¹H-¹³C correlation spectrum (high resolution) of the 12 peracetate.

FIG. 15 is the NOESY correlation spectrum of the H2 peracetate.

FIG. 16 is the NOESY correlation spectrum of the H2 peracetate.

FIG. 17 is the NOESY correlation spectrum of the H2 peracetate.

FIG. 18 is the structure of H2 identified to be epicatechin-4β→8-epicatechin.

FIG. 19 is the ¹H NMR spectrum of peak H1.

FIG. 20 is the ¹³C NMR spectrum of peak H1.

FIG. 21 is the peracetate structure of a sample of pure H1 following acetylation.

FIG. 22 is the ¹H NMR spectrum of the H1 peracetate.

FIG. 23 is the ¹³C NMR spectrum of the H1 peracetate.

FIG. 24 is the CIGAR ¹H-¹³C correlation spectrum (low resolution) of the H1 peracetate.

FIG. 25 is the CIGAR ¹H-¹³C correlation spectrum (high resolution) of the H1 peracetate.

FIG. 26 is the structure of H1 identified to be catechin-4α→8-epicatechin.

FIG. 27 is a HPLC tracing demonstrating separation of peak K2.

FIG. 28 is a HPLC tracing demonstrating separation of a pure peak K2.

FIG. 29 is −ve ion electrospray mass spectrum of K2.

FIG. 30 is the ¹H NMR spectrum of K2.

FIG. 31 is the ¹H NMR spectrum of the K2 peracetate.

FIG. 32 is the ¹³C NMR spectrum of the K2 peracetate.

FIG. 33 is a CIGAR ¹H-¹³C correlation spectrum (low resolution) of the K2 peracetate.

FIG. 34 is a CIGAR ¹H-¹³C correlation spectrum (high resolution) of the K2 peracetate.

FIG. 35 is the peracetate structure of K2.

FIG. 36 is the structure of K2 determined to be epicatechin-4β→4-epicatechin-4β→-epicatechin.

FIG. 37 is a graph of a Thioflavin T fluorometry assay demonstrating the dose-dependent disruption/disassembly of pre-formed Aβ 1-42 fibrils by proanthocyanidins (compounds H2, H1 and K2).

FIG. 38 is a black and white figure of a SDS-PAGE/Western blot further demonstrating the disruption of Aβ 1-42 fibrils, even in monomeric form by proanthocyanidins (compounds H2, H1 and K2).

FIG. 39 is a graph of a circular dichroism spectroscopy assay demonstrating compound H2 (referred to as PTC38 in this figure) causes a marked disruption/disassembly of β-sheet structure in Aβ 1-42 fibrils at 7 days following incubation.

FIG. 40 is a graph of a circular dichroism spectroscopy assay demonstrating compound H2 (referred to as PTC38 in this figure) causes a marked disruption/disassembly of β-sheet structure in Aβ 1-40 fibrils at 7 days following incubation.

FIG. 41 is a graph of a Thioflavin T fluorometry assay demonstrating the dose-dependent disruption/disassembly of pre-formed NAC fibrils by proanthocyanidins (compounds H2, H1 and K2).

FIG. 42 is a graph of a Thioflavin T fluorometry assay demonstrating the dose-dependent disruption/disassembly of pre-formed IAPP fibrils by proanthocyanidins (compounds H2, H1 and K2).

FIG. 43 is the peracetate structure of K1.

FIG. 44 is the structure of K1 determined to be epiafzelechin-4β→8-epicatechin.

FIG. 45 is the ⁻ve ion electrospray mass spectrum of K1.

FIG. 46 is the ¹³C NMR spectrum of K1.

FIG. 47 is the ¹H NMR spectrum of K1.

FIG. 48 is the ¹H NMR spectrum of the K1 peracetate.

FIG. 49 is the ¹³C spectrum of the K1 peracetate.

FIG. 50 is the CIGAR ¹H-¹³C correlation spectrum (low resolution) of the K1 peracetate.

FIG. 51 is the CIGAR ¹H-¹³C correlation spectrum (medium resolution) of the K1 peracetate.

FIG. 52 is the CIGAR ¹H-¹³C correlation spectrum (high resolution) of the K1 peracetate.

FIG. 53 is the CIGAR ¹H-¹³C correlation spectrum (high resolution) of the K1 peracetate.

FIG. 54 is an illustration of general Formula I for the structure of proanthocyanidins.

FIG. 55 is an illustration of general Formula II for the structure of proanthocyanidins.

FIG. 56 is an alternate example of proanthocyanidin structure.

FIG. 57 is a flowchart of an isolation process for proanthocyanidins.

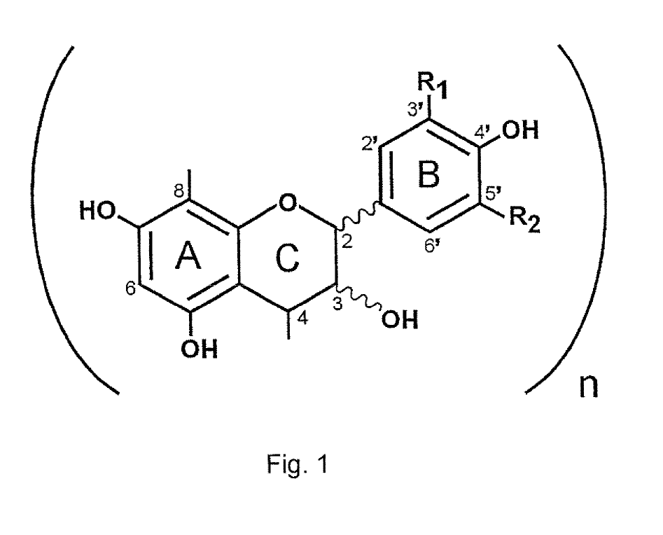

### BEST MODE OF CARRYING OUT THE INVENTION

### Further Definitions

In this disclosure, the following terms shall have the following meanings, without regard to whether the terms are used variantly elsewhere in the literature or otherwise in the known art.

“Proanthocyanidins” includes “procyanidins”; “procyanidins” are a specific class of “proanthocyanidins”.

“Mammal” and “mammalian subject” includes, but is not limited to, humans and non-human mammals, such as companion animals (cats, dogs, and the like), lab animals (such as mice, rats, guinea pigs, and the like) and farm animals (cattle, horses, sheep, goats, swine, and the like).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

“Pharmaceutically acceptable salts” means salts that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Such salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). When there are two acidic groups present, a pharmaceutically acceptable salt may be a mono-acid-mono-salt or a di-salt; and similarly where there are more than two acidic groups present, some or all of such groups can be salified.

A “therapeutically effective amount” in general means the amount that, when administered to a subject or animal for treating a disease, is sufficient to effect the desired degree of treatment for the disease. A “therapeutically effective amount” or a “therapeutically effective dosage” preferably inhibits, reduces, disrupts, disassembles amyloidosis, fibril formation, deposition, accumulation and/or persistence, or a disease associated with α-synuclein/NAC fibril formation in a patient by at east 20, more preferably by at least 40%, even more preferably by at least 60%, and still more preferably by at least 80%, relative to untreated subjects. Effective amounts of a proanthocyanidin or procyanidin, or other disclosed compositions for treatment of a mammalian subject are about 1 mg to about 10,000 mg/kg of body weight of the subject, but more preferably from about 10 mg/kg/body weight to 100 mg/kg body weight. A broad range of disclosed composition dosages are believed to be both safe and effective.

“Treating” or “treatment” of a disease includes preventing the disease from occurring in a mammal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). “Treating” amyloidosis or “amyloid diseases” includes any one or more of the following: preventing, inhibiting, reducing, disassembling, disrupting, and disaggregating amyloid fibrils and amyloid protein deposits, such as Aβ and the other amyloids referred to herein.

“Treating” an α-synuclein disease or “treating α-synuclein or NAC fibrillogenesis” includes any one or more of the following: preventing, inhibiting, reducing, disassembling, disrupting, and disaggregating α-synuclein/NAC fibrils and α-synuclein/NAC-associated protein deposits, such as those in Lewy body disease, Parkinson's disease and multiple system atrophy.

“NAC” (non-amyloid component) is a 35-amino acid peptide fragment of α-synuclein, which also, like α-synuclein, has the ability to form amyloid-like fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. U.S.A 90:11282-11286, 1993). Inhibition of NAC fibril formation, deposition, accumulation, aggregation, and/or persistence is believed to be effective treatment for a number of diseases involving α-synuclein, such as Parkinson's disease, Lewy body disease and multiple system atrophy.

“Fibrillogenesis” refers to the presence of amyloid fibrils or fibrils formed containing α-synuclein and/or NAC. Inhibition of such fibrillogenesis with a therapeutic compound may include, but not limited to, treating, inhibiting, preventing, or managing such amyloid, amyloid fibril, α-synuclein and/or NAC fibril formation, deposition, accumulation, aggregation and/or persistence in a mammalian subject.

“A pharmaceutical agent” or “pharmacological agent” or “pharmaceutical composition” refers to a compound or combination of compounds used for treatment, preferably in a pure or near pure form. In the specification, pharmaceutical or pharmacological agents include the proanthocyanidins and procyanidins as examples. Disclosed pharmaceutical or pharmacological compounds or compounds in compositions, are purified to 80% homogeneity, and preferably 90% homogeneity. Compounds and compositions purified to 99.9% homogeneity are believed to be advantageous. As a test or confirmation, a pure compound on HPLC would yield a single sharp-peak band.

The disclosed compounds and compositions may possess one or more chiral centers, and can therefore be produced as individual stereoisomers or as mixtures of stereoisomers, depending on whether individual stereoisomers or mixtures of stereoisomers of the starting materials are used. Unless indicated otherwise, the description or naming of a compound or group of compounds is intended to include both the individual stereoisomers and mixtures (racemic or otherwise) of stereoisomers. Methods for the determination of stereochemistry and the separation of stereoisomers are well known to a person of ordinary skill in the art [see the discussion in Chapter 4 of March J: Advanced Organic Chemist, 4th ed. John Wiley and Sons, New York, N.Y., 1992].

“Optionally substituted glycosyl” is glycosyl optionally substituted with up to three anionic substituents selected from sulfate, sulfonate, phosphate, phosphonate, and carboxylate, each optionally esterified with optionally substituted alkyl, optionally substituted aryl, or optionally substituted heteroaryl; examples include glucosyl, galactosyl, rhamnogalactosyl, and the like.

“Aryl” is a cyclic (monocyclic, condensed bicyclic, or linked bicyclic) group having from 5 to 12 ring carbon atoms, and sufficient ring unsaturation that the group is “aromatic” as that term is conventionally used, e.g. phenyl, naphthyl, biphenylyl, and the like. A “heteroaryl” group is an “aryl” group as just defined in which from 1 to 4 of the ring carbon atoms have been replaced by O, S or NR (where R is hydrogen or C₁₋₆ alkyl), e.g. pyrrolyl, furanyl, thienyl, benzofuranyl, and the like. A “substituted aryl or heteroaryl” is an aryl or heteroaryl group as just defined substituted by 1 to 3, preferably adjacent, hydroxyl groups, and up to 5 non-interfering substituents. A non-interfering substituent is a substituent that does not adversely affect the pharmacological activity of the compound and is not otherwise pharmacologically undesirable. Suitable non-interfering substituents include halogen, and C₁₋₆ alkyl and C₁₋₄ alkoxy, each optionally substituted with up to five halogen atoms.

Disclosed compounds for pharmacological or pharmaceutical treatment of an amyloid disease, or for treatment of α-synuclein/NAC fibrillogenesis, will include and not be limited to proanthocyanidins, procyanidins, anthocyanins, condensed tannins, leucoanthocyanidins, leucocyanins, anthocyanogens, epicatechin-catechin polymers or oligomers, flavanoids, flavan-3,4-diols, propelargonidins, and A-type, B-type and C-type procyanidins.

It has now been surprisingly found that Uncaria tomentosa compounds and extracts exhibit potent anti-amyloid activity. The individual compounds found to exhibit such activity belong to the general class of compounds known as polyphenols, and more specifically procyanidins and proanthocyanidins. Disclosed are methods involving the steps of isolation to acquire individual compounds of the invention including, but not limited to procyanidins B2, B4 and C1. The extracts and compounds having anti-amyloid and anti-α-synuclein/NAC inhibitory activity can be purified by a variety of methods disclosed herein, including solvent extraction techniques, get permeation chromatography, preparative high performance liquid chromatography or a combination of such techniques.

Anti-amyloid and anti-α-synuclein/NAC compositions and compounds containing the proanthocyanidins can be prepared in accordance with standard techniques well known to those skilled in the pharmaceutical art. Such compositions can be administered in dosages and by techniques well known to those skilled in the art taking into consideration such factors such as age, sex, weight, and condition of the particular patient, and the route of administration. The compositions can be co-administered or sequentially administered with other potential anti-amyloid agents, or anti-α-synuclein/NAC agents; again taking into consideration such factors as the age, sex, weight and condition of the particular patient, and the route of administration.

Examples of compositions useful to effect the disclosed aims include solid compositions for oral administration such as capsules, tablets, pills, and the like, as well as chewable solid formulations, to which the present invention may be well suited; liquid preparations for orifice, e.g. oral, nasal, administration such as suspensions, syrups or elixers; and preparations for parental, subcutaneous, intradermal, intramuscular or intravenous administration (e.g. injectable administration) such as sterile suspensions or emulsions. The active proanthocyanidin compound may be in admixture with a suitable carrier, diluent, or excipient such as sterile water, physiological saline or the like. The active anti-amyloid compounds of the invention can be provided in lyophilized form for reconstituting, for instance, in isotonic, aqueous, saline buffer.

These compounds can be purified, e.g., compounds or combinations thereof can be substantially pure; for instance, purified to apparent homogeneity. Purity is a relative concept, and the numerous Examples demonstrate isolation of inventive compounds or combinations thereof, as well as purification thereof, such by the methods exemplified a skilled artisan can obtain a substantially pure compound or combination thereof, or purify them to apparent homogeneity (e.g., purity by HPLC; observation of a single chromatographic peak). As defined herein, a substantially pure compound or combination of compounds is at least about 70% pure, more advantageously at least 80-% pure, at least 90% pure, more preferably greater than 90% pure, e.g., at least 90-95% pure, or even purer such as greater than 95% pure, e.g., 99.99% pure.

Polyphenols, (+)-catechin and (−)-epicatechin, are used herein to exemplify the types of polyphenol oligomers that may be prepared by the method of the present invention. The linkages between adjacent, the polyphenol monomers, (+)-catechin and (−)-epicatechin, are from position 4 to position 6 or from position 4 to position 8; and this linkage between position 4 of a monomer and position 6 and 8 of the adjacent monomeric units is designated herein as (4→6) or (4→8).

Moreover, stereoisomers of the oligomers are encompassed within the scope of the invention. The stereochemistry of the substituents on a flavanoid monomer of the oligomer may be described in terms of their relative stereochemistry, “alpha/beta” or “cis/trans”, or in the terms of the absolute stereochemistry, R/S. The term “alpha” indicates that the substituent is oriented below the plane of the flavan ring, whereas “beta” indicates that the substituent is oriented above the plane of the ring. The term “cis” indicates that the two substituents are oriented on the same face of the ring, whereas “trans” indicates that the two substituents are oriented on opposite faces of the ring. The terms R and S are used to denote the arrangement of the substituents about a sterogenic or “chiral” center, based on the ranking of the groups according to the atomic number of the atoms directly attached to that stereogenic center. For example, the polyphenol, (+)-catechin, may be defined as (2R,trans)-2-(3′,4′-dihydroxyphenyl)-3,4-dihydo-2H-1-benzopyran-3,5,7-triol, or as (2R,3S)-flavan-3,3′,4′,5,7-pentaol. Interflavan (polyphenol-polyphenol) bonding is often characterized using the relative terms α/β or cis/trans; α/β is used herein to designate the relative stereochemistry of the interflavan bonding.

There are multiple stereochemical linkages between position 4 of a monomer and position 6 and 8 of the adjacent monomer; and the stereochemcial linkages between monomeric units is designated as (4α→6) or (4β→6) or (4α→8) or (4β→8) for linear oligomers. When catechin is linked to another catechin or epicatechin, the linkages are advantageously (4α→6) or (4β→8). When epicatechin is linked to catechin or another epicatechin, the linkages are advantageously (4β→6) or (4β→8).

In addition to carbon position 4, a bond to carbon position 2 has alpha or beta stereochemistry, and a bond to carbon position 3 has alpha or beta stereochemistry (e.g., (−)-epicatechin or (+)-catechin).

Examples of preferred compounds include, but are not limited to, dimers, epicatechin-4β→8-epicatechin and epicatechin-4β→6-epicatechin, wherein epicatechin-4β→8-epicatechin is preferred; trimers, [epicatechin-(4β→8)]₂-epicatechin, [epicatechin-(4β→8)]₂-catechin and [epicatechin-(4β6→)]₂-epicatechin, wherein [epicatechin-(4β→8)]₂-epicatechin is preferred; tetramers, [epicatechin-(4β→8)]₃-epicatechin; [epicatechin-(4β→8)]₃-catechin; and [epicatechin-(4β→8)]₂-epicatechin-(4β→6)-catechin, wherein [epicatechin-(4β→8)]₃-epicatechin is preferred; and pentamers, [epicatechin-(4β→8)]₄-epicatechin; [epicatechin-(4β→8)]₃-epicatechin-(4β→6)epicatechin; [epicatechin-)(4β→8)]₃-epicatechin-(4β→6)-catechin; [epicatechin-(4β→8)]₃-epicatechin0(4β→8)-catechin; and [epicatechin-(4β→8)]₃-epicatechin-(4β→6)-catechin, wherein [epicatechin-(4β→8)]₄-epicatechin is preferred.

It will be understood from the detailed description that the aforementioned list is exemplary and is provided to illustrate the types of compounds that may be prepared by the methods of the present invention and it is not intended as an exhaustive list of the inventive compounds encompassed by the present invention.

One skilled in the art will appreciate that rotation of a number of bonds within the oligomer of the invention may be restricted due to steric hindrance, particularly if the oligomer is substituted, such as with benzyl groups. Accordingly, all possible regioisomers and stereoisomers of the compounds of the invention are encompassed within the scope of the invention.

Proanthocyanidins can not only be extracted and purified from Uncaria tomentosa as described in the present invention, but also from other various plants such as grape, kaki (Japanese persimmon), betel palm, apple, barley, cocoa leaf, cocoa liqueur, dark chocolate, Nest-leaf, rhubarb, cinnamon, adzuki bean, raspberry, etc. They can also be obtained by conventional chemical synthesis.

Regarding chemical synthesis of procyanidins and proanthocyanidin, a method of producing dimers of epicatechin or catechin is disclosed in Journal of Chemical Society, Parkin Transaction I, pp. 1535-1543, 1983. To chemically produce proanthocyanidins for use as disclosed, is referred to in U.S. Pat. No. 6,165,912 (Tuckmantel et al; Dec. 5, 2000) and U.S. Pat. No. 6,207,842 B1 (Romanczyk Jr. et al; Mar. 27, 2001), which is incorporated herein.

Furthermore, while procyanidins or proanthocyanidins derived from Uncaria tomentosa art disclosed, persons skilled in the art will appreciate by means of this disclosure and envision synthetic and alternate extraction routes to obtain the active compounds. Accordingly, synthetic polyphenols or procyanidins or proanthocyanidins or their derivatives which include, but are not limited to glycosides, gallates, esters, and the like are included within the scope of the invention.

Disclosed are methods pertaining to the isolation, identification and use of anti-amyloid compounds derived from plant material, and the surprising discovery that proanthocyanidins are potent inhibitors of amyloid and α-synuclein/NAC fibrillogenesis, and cause a potent disruption/disassembly of pre-formed fibrils for a variety of amyloid and α-synuclein diseases. Exemplary compounds identified to serve as potent amyloid fibril inhibiting agents include procyanidins, such as epicatechin-epicatechin, catechin-epicatechin, epiafzelechin-epicatechin dimers, epicatechin-epicatechin-epicatechin trimers, as well as other epicatechin and/or catechin oligomers for the treatment of amyloid diseases including, but not limited to, Alzheimer's disease, type II diabetes, and systemic AA amyloidosis, as well as inhibiting α-synuclein or non-amyloid component (NAC) fibril formation for the treatment of Parkinson's and Lewy body disease.

Also disclosed are methods for preparing and isolating such compounds, as well as new uses for them, especially as amyloid and α-synuclein/NAC fibril disrupting agents. This invention is also directed to methods for inhibiting or eliminating amyloid fibril formation, deposition, accumulation and/or persistence in a number of different amyloid diseases by treatment of patients with proanthocyanidins of the A, B and C types, including monomers, dimers, trimers and multimers of epicatechin and catechin. An exemplary procyanidin compound is a substituted epicatechin-epicatechin or catechin-epicatechin dimer, such as epicatechin-4β→8-epicatechin, catechin-4α→8-epicatechin, or epiafzelechin-4β→8-epicatechin, or other oligomers.

Methods of isolation, identification and use of amyloid-inhibiting compounds derived from plant material are disclosed for the therapeutic intervention of Alzheimer's disease, type 2 diabetes, Parkinson's disease, systemic AA amyloidosis and other diseases involving amyloid fibril formation and accumulation, especially methods of isolating amyloid inhibiting compounds from Uncaria tomentosa and related plants, and to the use of those compounds,

### Pharmacology and Utility

The disclosed compounds act to inhibit or prevent amyloid fibril formation, inhibit or prevent amyloid fibril growth, and/or cause disassembly, disruption, and/or disaggregation of preformed amyloid fibrils and amyloid protein deposits. Their activity can be measured in vitro by methods such as those discussed in Examples 4 through 7 while their activity in vivo against amyloidoses can be measured in animal models, such as those of Alzheimer's disease and in humans by a method such as that discussed in Example 11.

The disclosed compounds also act to inhibit or prevent α-synuclein/NAC fibril formation, inhibit or prevent α-synuclein/NAC fibril growth, and/or cause disassembly, disruption, and/or disaggregation of preformed α-synuclein/NAC fibrils and α-synuclein/NAC-associated protein deposits. Their activity can be measured in vitro by methods similar to those discussed in Examples 4 through 7 below.

The therapeutic ratio of a compound can be determined, for example, by comparing the dose that gives effective anti-fibril (anti-amyloid or anti-α-synuclein/NAC activity in a suitable in vivo model in a suitable animal species such as the mouse, with the dose that gives significant weight loss (or other observable side-effects) in the test animal species.

### Pharmaceutical Compositions and Administration

In general, compounds will be administered in pure isolated form in therapeutically effective amounts by any of the usual modes known in the art, either singly or in combination with at least one other compound of this invention and/or at least one other conventional therapeutic agent for the disease being treated. A therapeutically effective amount may vary widely depending on the disease, its severity, the age and relative health of the animal being treated, the potency of the compound(s), and other factors. As anti-fibril agents, therapeutically effective amounts of compounds of this invention may range from 1-1000 mg/Kg body weight; for example, 10-100 mg/Kg. A person of ordinary skill in the art will be conventionally able, and without undue experimentation, having regard to that skill and to this disclosure, to determine a therapeutically effective amount of a compound for the treatment of amyloidosis or α-synuclein/NAC fibril formation.

In general, compounds will be administered as pharmaceutical compositions by one of the following routes: oral, topical, systemic (e.g. transdermal, intranasal, or by suppository), or parenteral (e.g. intramuscular, subcutaneous, or intravenous injection). Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Alfonso A R: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols.

In particular, the compound(s)—optimally only one such compound is administered in any particular dosage form—can be administered, orally, for example, as tablets, troches, lozenges, aqueous or oily suspension, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the compound in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, maize starch or alginic acid; binding agents, for example, maize starch, gelatin or acacia, and lubricating agents, for example, magnesium stearate or stearic acid or tale. The tablets may be uncoated or they may be coated by known techniques to delay disintegration and absorption in the gastrointestinal tract and thereby provide a sustained action over a longer period. For example, a time delay material such as glycerol monostearate or glycerol distearate may be employed. Formulations for oral use may also be presented as hard gelatin capsules wherein the compound is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Aqueous suspensions contain the compound in admixture with excipients suitable for the manufacture of aqueous suspensions. Such excipients are suspending agents, for example, sodium carboxymethylcellulose, methylcellulose, hydroxypropylmethyl cellulose, sodium alginate, polyvinylpyrrolidone, gum tragacanth and gum acacia; dispersing or wetting agents may be naturally occurring phosphatides, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids such as hexitol such as polyoxyethylene sorbitol monooleate, or condensation products of ethylene oxide with partial esters from fatty acids and a hexitol anhydrides, for example, polyethylene sorbitan monooleate. The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the compound in a vegetable oil, for example arachis oil, olive oil, sesame oil, or coconut oil or in a mineral oil such as liquid paraffin. The oily suspensions may contain a thickening agent, for example beeswax, hard paraffin or cetyl alcohol. Sweetening agents, such as those set forth below, and flavoring agents may be added to provide a palatable oral preparation. These compositions may be preserved by the addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already described above. Additional excipients, for example sweetening, flavoring and agents, may also be present.

The compounds may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occurring phosphatides, for example soy bean, lecithin, and occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsion may also contain sweetening and flavoring agents. Syrups and elixirs may be formulated with sweetening agents, for example, glycerol, sorbitol or sucrose. Such formulations may also contain a demulcent, a preservative and flavoring and coloring agents.

The compound can also be administered by injection or infusion, either subcutaneously or intravenously, or intramuscularly, or intrasternally, or intranasally, or by infusion techniques in the form of sterile injectable or oleaginous suspension. The compound may be in the form of a sterile injectable aqueous or oleaginous suspensions. These suspensions may be formulated according to the known art using suitable dispersing of wetting agents and suspending agents that have been described above. The sterile injectable preparation may also be a sterile injectable solution or suspension in a non-toxic parenterally-acceptable diluent or solvent for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oils may be conventionally employed including synthetic mono- or diglycerides. In addition fatty acids such as oleic acid find use in the preparation of injectables. Dosage regimens can be adjusted to provide the optimum therapeutic response. For example, several divided dosages may be administered daily or the dosage may be proportionally reduced as indicated by the exigencies of the therapeutic situation.

It is especially advantageous to formulate the compounds in dosage unit form for ease of administration and uniformity of dosage. Dosage unit form as used herein refers to physically discrete units suited as unitary dosages for the subjects to be treated; each containing a therapeutically effective quantity of the compound and at least one pharmaceutical excipient. A drug product will comprise a dosage unit form within a container that is labeled or accompanied by a label indicating the intended method of treatment, such as the treatment of an amyloid disease, such as Alzheimer's disease, or of a disease associated with α-synuclein/NAC fibril formation, such as Parkinson's disease.

The following non-limiting Examples are given by way of illustration only and are not considered a limitation of this invention, many apparent variations of which are possible without departing from the spirit or scope thereof.

### EXAMPLES

### Example 1

### Isolation of Amyloid Inhibitory Components from Uncaria tomentosa and PTI-777

We have previously reported in U.S. application Ser. No. 09/753,313 filed Dec. 29, 2000, U.S. application Ser. No. 09/938,987 filed Aug. 24, 2001, U.S. application Ser. No. 60/271,777 filed Feb. 27, 2001, and U.S. application Ser. No. 60/338,721 filed Nov. 2, 2001 pertaining to the discovery of amyloid inhibitory components in an extract of the rain forest woody vine, Uncaria tomentosa, with regards to the treatment of neurological disorders involving beta-amyloid protein (Aβ) or α-synuclein/NAC fibrillogenesis and other amyloid disorders. We have previously reported the discovery that a methonolic extract of the powdered bark of Uncaria tomentosa contains potent amyloid inhibitory activity that is relatively concentrated in a mixture of compounds consisting of mainly polyphenols. Tests of samples of pure oxindole alkaloids that were known to be major components of Uncaria tomentosa also demonstrated in these previous studies that oxindole alkaloids were not responsible for the amyloid inhibitory activity.

Previously we isolated and identified two polyphenolic compounds from the methanolic extract of Uncaria tomentosa known as PTI-777; and these were demonstrated to be chlorogenic acid and epicatechin. The methanolic extract of Uncaria tomentosa found to exhibit potent amyloid inhibitory activity was previously referred to as “PTI-777”. As described in previously reported pending U.S. patent application Ser. No. 60/271,777 filed Feb. 27, 2001, PTI-777 represents a group of approximately 11 major fractions referred to as fraction F, fraction G, fraction H, fraction I, fraction J, fraction K1, fraction K2, fraction L, fraction M, fraction N and fraction O, which were isolated from the powdered bark of Uncaria tomentosa. Some of these fractions, as demonstrated in the present invention, were further purified to one or two major components as was done with fraction H (now found to contain 2 major components referred to as H1 and H2 as described below).

Using PTI-777 as a starting point, as described in pending U.S. patent application Ser. No. 60/271,777 filed Feb. 27, 2001, we disclose details of methods of isolation and identification of further major components contained within PTI-777, referred to as compounds H2, H1, K2 and K1, which all belong to the general class of proanthocyanidins, and which were all found to possess potent amyloid and α-synuclein/NAC inhibitory activity. In addition, we disclose and teach new methods for the isolation of such anti-amyloid/anti-α-synuclein/NAC compounds, and proanthocyanidins from Uncaria tomentosa, and other plants.

Varying the methods of high pressure liquid chromatography (HPLC) earlier reported by us in our applications cited above, and by using a lower % of acetonitrile (de-scribed in detail below), we obtained a trace where the main peak H, came off at the same relative retention time as earlier reported, and which also resulted in a further separation of some of the peaks (i.e. H1 and H2), previously seen to be overlapping (i.e. as fraction H). In a further variation, and noting that one of our HPLC traces was run at 1.5 min/ml, we found that running the elution at 2 ml/min obtained a trace almost identical to the 1.5 ml/min trace.

Epicatechin, together with catechin, epigallocatechin and various catechin gallates are known to be present in green tea (See our U.S. patent application Ser. No. 09/753,313, filed Dec. 29, 2000; and also Baumann et al., J. Natural Prod, 64:353-355, 2001, and Zeeb et al, Anal. Chem. 72:5020-5026, 2000). In some of the reports of the isolation of some of these catechins, sephadex LH20 gel in particular, along with other gels, and also solvent partitions, and other HPLC methods have been discussed. In addition, silica gel chromatography, eluting with a gradient of methanol in chloroform has also been used on compounds of a polarity similar to the catechins, for example, flavanoid and iridoid glycosides (Kim et al, J. Nat. Prods. 64:75-78, 2001; Sang et al, J. Nat. Pods. 64:799-800, 2001; Calis et al, J. Nat. Prods. 64:961-964, 2001).

We had already used a Sephadex LH20 gel in an earlier step in the purification of the extract PTI-777. Further work was therefore concentrated on the use of reverse phase (RP) C18 silica or silica gel chromatography. Using a variety of solvent systems, we analyzed the use of RP C18 and silica gel thin layer chromatography (TLC) behavior of the methanol extract of PTI-777. RP C18 silica showed all the material to be in one main spot on the TLC, but with silica we could see several separate spots.

We therefore tried separation of a small sample of extract PTI-777 by silica gel chromatography. The extract appeared to be both light and air sensitive, so the column was used in limited light, with a rapid solvent gradient, to minimize decomposition on the silica. HPLC analysis of the column fractions showed that we had discovered new methods to perform good separation.

In particular fraction 9 (see methods 1 and 2) was almost pure epicatechin, and fractions 11 to 13 contained mostly peak H12, with its underlying H1 peak. Other fractions showing interesting concentrations of other peaks, in particular K1 was concentrated in fraction 10, whilst K2 was concentrated in fraction 14. The total recovery of useful material was 50%, but given the separation of some of the peaks, this appeared to be a good way to separate a large amount of material to give fractions rich in various peaks.

### Example 2

### Isolation and Identification of Peak H2 from PTI-777 as an Epicatechin-Epicatechin Dimer

### General Experimental Procedures

All solvents were distilled before use and were removed by rotary evaporation under vacuum at temperatures up to 20-60°. Octadecyl functionalised silica gel (C18) was used for reversed-phase (RP) flash chromatography, and Merck silica gel 60, 2-400 mesh, 40-63 μm, was used for silica gel flash chromatography. TLC was carried out using Merck DC-plastikfolien Kieselgel 60 F₂₅₄, first visualised with a UV lamp, and then by dipping in 5% aqueous ferric chloride solution. Optical rotations were measured on a Perkin-Elmer 241 polarimeter. Mass, ultraviolet (UV), and infra-red (IR) spectra were recorded on Kratos MS-80, Shimadzu UV 240, and Perkin-Elmer 1600 FTIR instruments, respectively, NMR spectra, at 25-, were recorded at 500 or 300 MHz for ¹H and 125 or 75 MHz for ¹³C on Varian INOVA-500 or VXR-300 spectrometers. Chemical shifts are given in ppm on the δ scale referenced to the solvent peak CH₃OH at 3.30, CD₃OD at 49.3 ppm, CHCl₃ at 7.25, CDCl₃ at 77.0; (CH₃)₂CO at 2.15 and (CD₃)₂CO at 30.5.

### HPLC Conditions to Isolate Peaks H2 and H1

The analytical HPLC equipment consisted of a Waters 717 autosampler, 600 pump and controller, and a 2487 UV detector controlled by Omega software. Samples were analyzed by using an RP-118 semi-preparative column (Phenomenex Jupiter 5 μm C18 300A, 250×10 mm) with a guard column (Phenomenex SecurityGuard cartridge containing a C18 ODS 4×3 mm, 5 μm column) fitted at 30° C. Samples (5 μl) were analyzed using a mobile phase flow rate of 5.0 mL/min, with UV detection at 280 nm.

Solvent A—CH₃CN containing 0.1% TFA
Solvent B—H₂O containing 0.1% TFA

| | | |
|---|---|---|
| Time (minutes) | Solvent A | Solvent B |

HPLC Method 1

| | | |
|---|---|---|
| 0 | 11 | 89 |
| 20 | 11 | 89 |
| 30 | 100 | 0 |
| 31 | 11 | 89 |

HPLC Method 2

| | | |
|---|---|---|
| 0 | 8 | 92 |
| 20 | 8 | 92 |
| 30 | 100 | 0 |
| 31 | 8 | 92 |

Samples were generally run under HPLC method I, unless otherwise stated.

### An Example of the Silica Gel Fractionation of PTI-777

A sample of the extract PTI-777 (1 g) was dissolved in methanol (2 ml) then loaded onto a silica gel (10 g) column, prepared in chloroform. Elution of this column with increasing proportions of methanol in chloroform gave 45 fractions

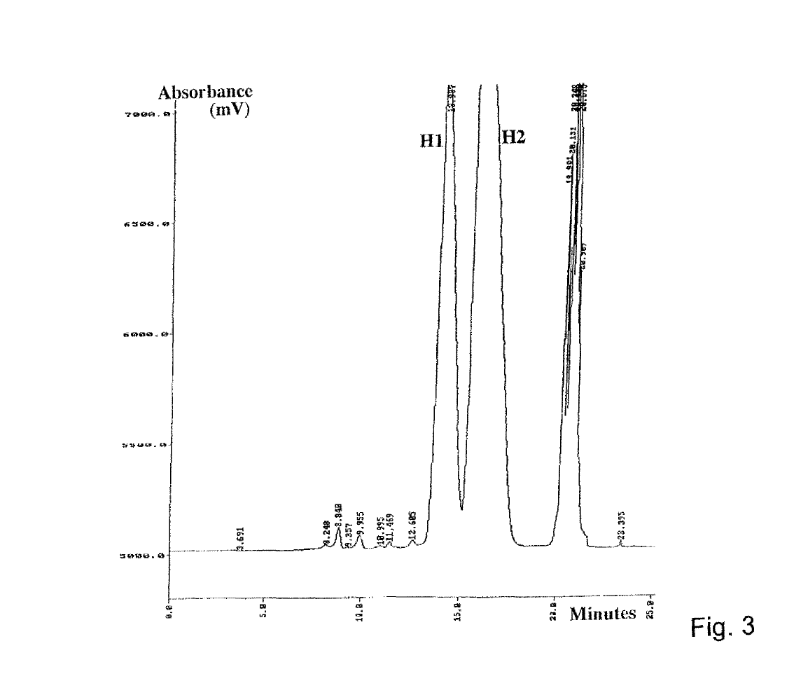

TABLE 1
Example of a Silica Gel Column Fractionation of PTI-777

| Solvent | Fractions | Peaks present | Weight | |
|---|---|---|---|---|
| 10% MeOH in | 1-11 | nothing | 0 mg | |
| CHCl₃ 50 ml | | | | |
| | 12-15 | J and pre-F | 45 mg | |
| | 16-19 | J, K1 | 16 mg | |
| 20% MeOH | 20-24 | H1, H2 | 134 mg | FIG. 3 |
| in CHCl₃ 50 ml | | | | |
| | 25-30 | H1, H2, K2 | 101 mg | |
| 40% MeOH in | 31-34 | H1, H2 mostly K2 | 39 mg | |
| CHCl₃ 50 ml | | | | |
| 50% MeOH in | 35-40 | H1, H2, K2, L post L | 226 mg | |
| CHCl₃ 50 ml | | | | |
| 100% MeOH | 41-45 | H1, H2, K2, others | 228 mg | |
| 100 ml | | | | |

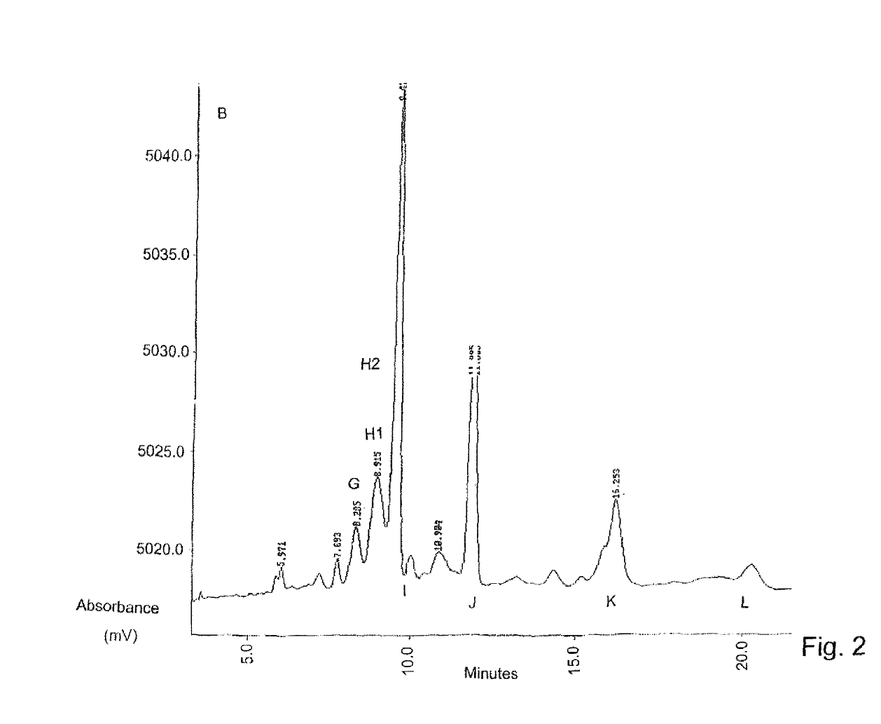

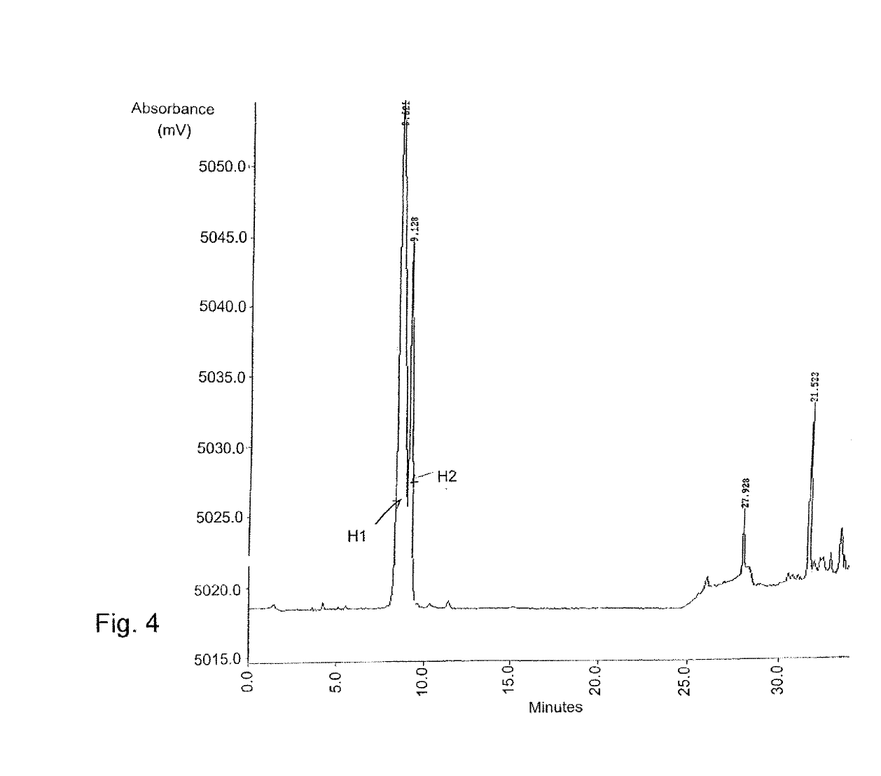

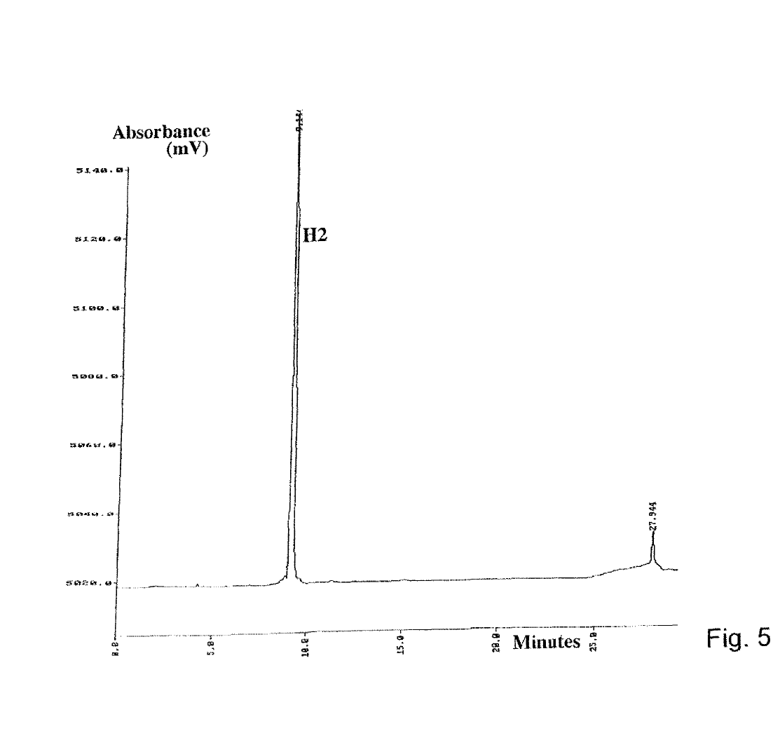

The main component of peak H, called H2, of the PTI-777 was isolated by a series of chromatographic techniques, monitored by HPLC (FIGS. 2-5). We initially separated the PTI-777 extract by column chromatography over silica gel (Table 1)(and tracings were monitored by HPLC using method 1), whereby elution with 20% methanol in chloroform gave a fraction rich in the two components of peak H (134 mg)(with the HPLC tracing using method 2, shown in FIG. 3). An HPLC method was developed to separate the two main components of peak H on a preparative scale (i.e. HPLC method 2), to give us a mostly pure H1 (16 mg)(HPLC method 1, FIG. 4) and pure H2 (23 mg) (HPLC method 1, FIG. 5).

A ⁻ve ion electrospray mass spectrum of H2 gave a clean 100% ion at 577 daltons. This is appropriate for the molecular ion (M⁺−H) of a molecular formula of C₃₀H₂₆O₁₂, such as a dimer of two epicatechin, or isomeric units. Epicatechin has previously been isolated from the PTI-777 extract and is described in the parent case to this application.

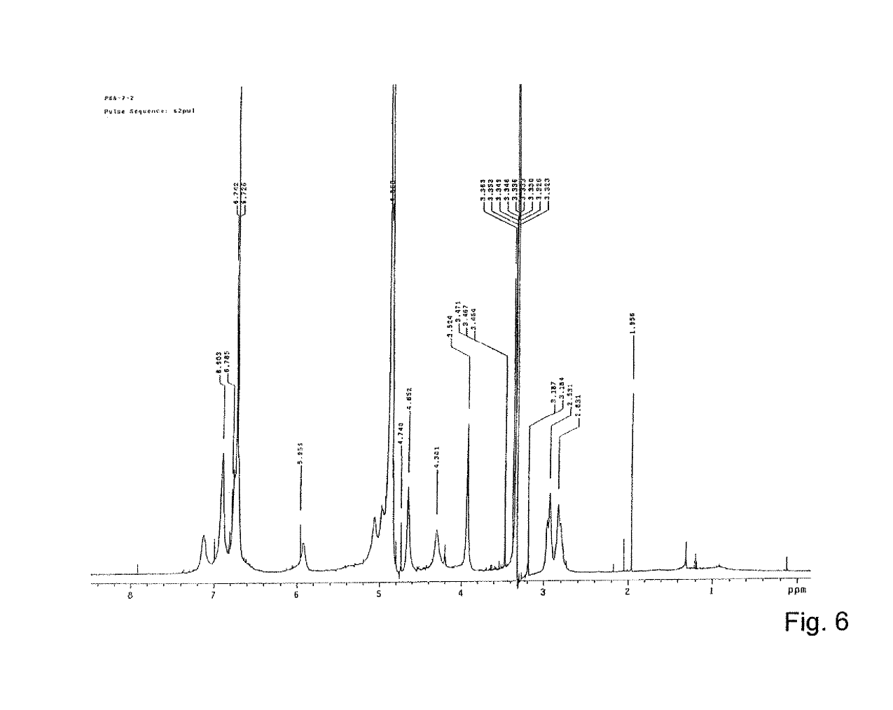

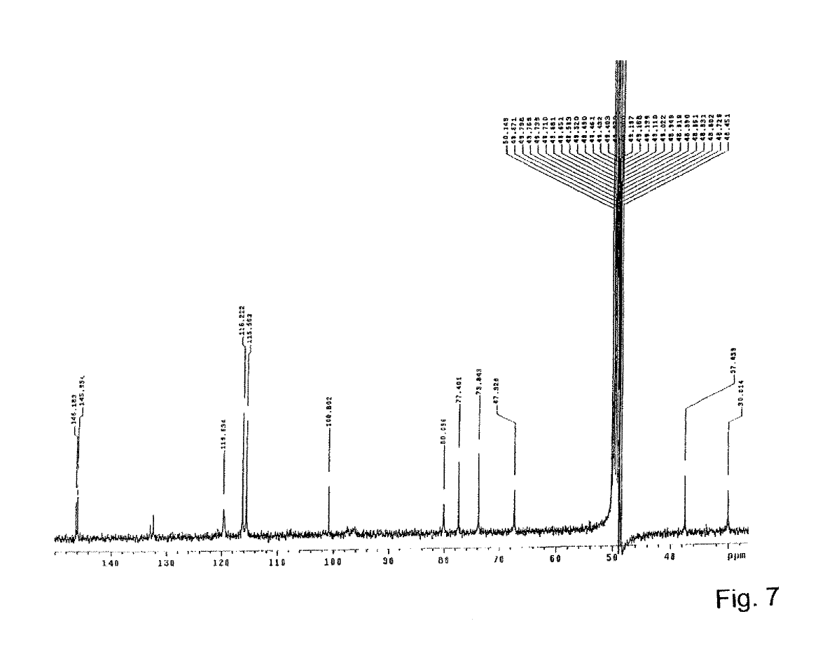

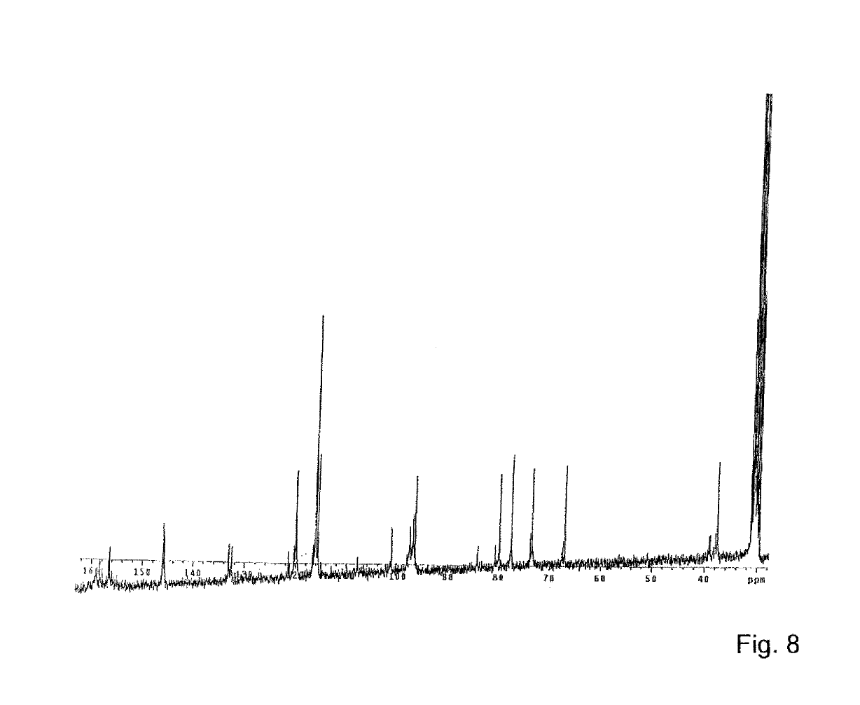

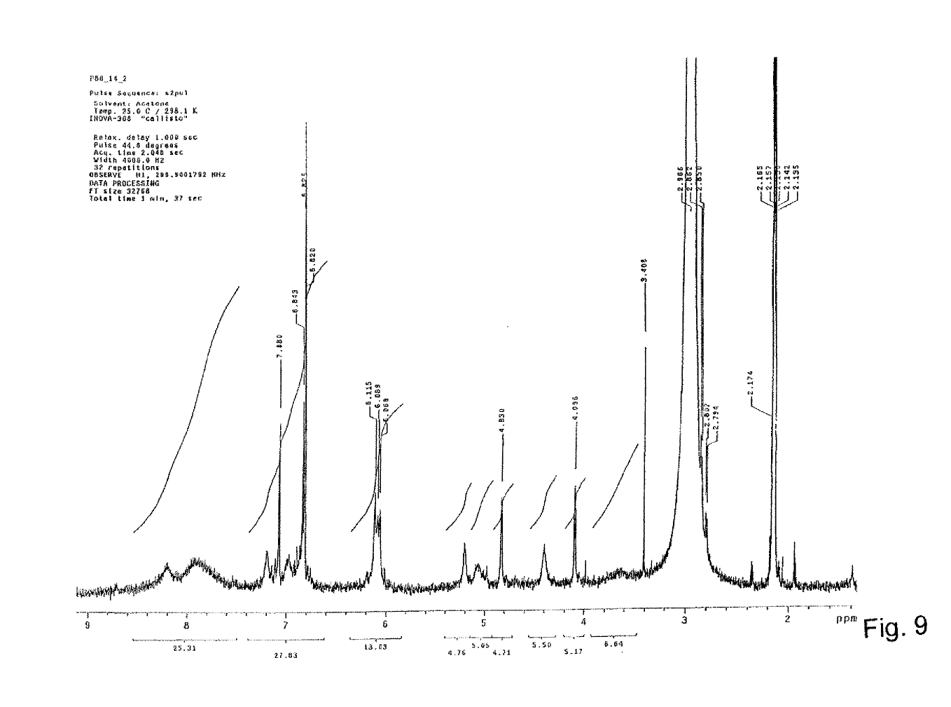

A ¹H NMR spectrum (FIG. 6) of peak H2 showed unusual broadening of the signals, whilst the ¹³C NMR (FIG. 7) showed sharp and broad signals, consistent with some kind of flavonol dimer. We were surprised to see no signals in the 5.8-6.3 ppm region of the ¹H spectrum, or in the 90-99 ppm region of the ¹³C NMR spectrum, where the characteristic H-6/H-8 and C-6/C-8 signals would appear, Running the NMR spectra in deuteroacetone (FIGS. 8, 9) instead of deuteromethanol, showed the expected signals to be present, indicating that in deuterated protic solvents, an exchange of these H-6 and H-8 protons for deuterons took place.

Broadening of signals is often due to restricted rotation within a molecule. This rotation can be sped up by running the spectrum at higher temperatures which gives sharper signals. We therefore ran the ¹H and ¹³C NMR spectra first at 40° C., then at 50° C. Unfortunately, although there were definite sips of sharpening of the signals, there were also new signals, showing either rearrangement or degradation of H2.

### Peak H2 Data Summary

Aliquots (14×70 μl) of fractions 20-24 (134 mg in 1 ml)(Table 1) from the above silica gel column were separated by HPLC using method 2. The peaks between 14.5 and 16.2 and between 16.2 and 19.0 minutes were collected, then freeze dried to give two products, about 80% pure H1, retention time 15.1 minutes (HPLC method 2)(16 mg) as a white solid; and pure Hz (23 mg) as a white solid, retention time 16.9 minutes (HPLC method 2).

−ve electrospray mass spectroscopy 577 (M⁺−H, 100%)

molecular weight of H2=578

¹H NMR (CD₃OD): 2.81 (1H, br d), 2.95 (1H, br d), 3.92 (1H, s), 4.30 (1H, br s), 4.65 (1H, br s), 4.98 (br s), 5.07 (1H, br s), 6.70-7.20 (6H, m).

¹³C NMR (CD₃OD): 29.97 (C-4l), 37.50 (C-4u), 67.33, 73.83, 77.45, 80.23, 101.00, 115.71, 116.27, 116.31, 119.77, 132.41, 145.96 and 146.22.

¹H NMR ((CD₃)₂CO), 2.83 (1H, m, H4l), 2.96 (1H, m, H4l), 4.10 (1H, s), 4.41 (1H, br s), 4.83 (1H, s, H-4u), 5.07 (1H, br s), 5.20 (1H, br s), 6.07 (1H, s), 6.09 (1H, s), 6.12 (1H, s), 6.80-7.20 (6H, m) and 7.50-8.30 (6H, br s, OHs)

¹³C NMR ((CD₃)₂CO): 30.3, 37.6, 67.1, 73.6, 77.6, 79.9, 96.6, 97.1, 977, 101.3, 115.5-116.2, 119.9, 120.2, 132.5, 133.1, 145.6-146.1, 156.4-160.0.

UV (MeOH) λ max (log ε) 210 (4.79), 227sh (4.48) and 280 (3.81) nm;

[∝]²⁴₅₈₉ₙₘ+29.0°, [∝]²⁴₅₇₇ₙₘ+19.8°, [∝]²⁴₅₄₆ₙₘ+5.7°, [∝]²⁴₄₃₅ₙₘ−87.4°, [∝]²⁴₄₀₅ₙₘ−106.1° (c 0.2, MeOH)

### Acetylation of H2

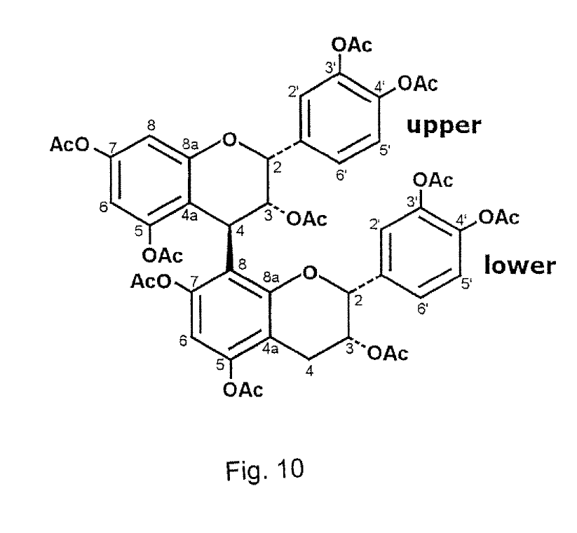

Since the compound H2 was unstable under the conditions necessary to ultimately prove its structure by NMR spectroscopy, we had to make a stable derivative. Acetylation of a sample of pure H2 gave a peracetate (FIG. 10), which was purified by column chromatography over silica gel. A larger sample of this peracetate, identical by NMR and TLC, was also obtained by silica gel separation of the two main products from acetylation of a fraction rich in H1 and H2.

For these studies, a sample of H2 (7 mg) was dissolved in a mixture of acetic anhydride (0.5 ml) and pyridine (0.5 ml). The mixture stood at room temperature for 18 hours, then the solvents were removed in vacuo. Purification by column chromatography over silica gel, eluting with 20% ethyl acetate in dichloromethane gave an H2 peracetate (6 mg) as a colorless gum. The NMR data is collated in Table 2.

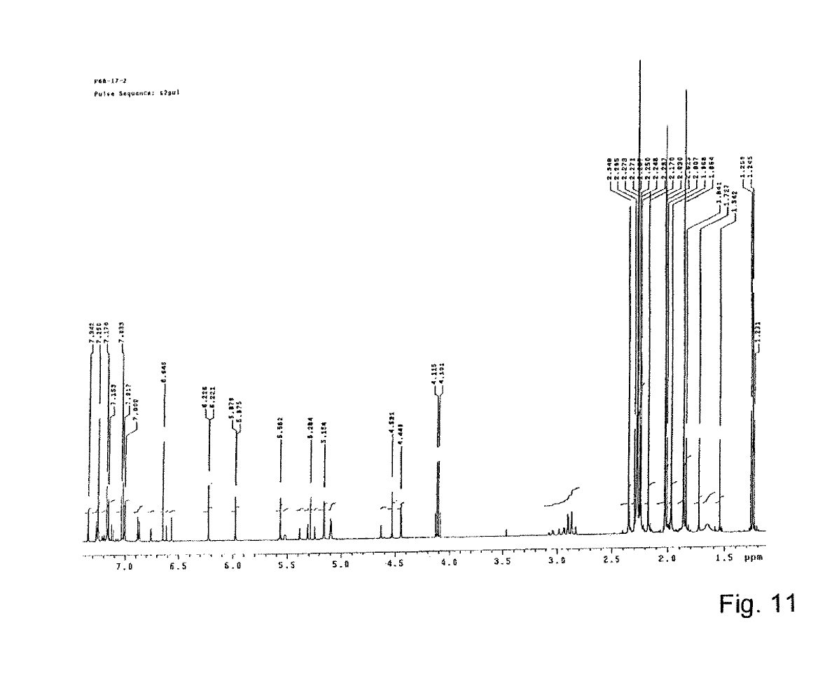

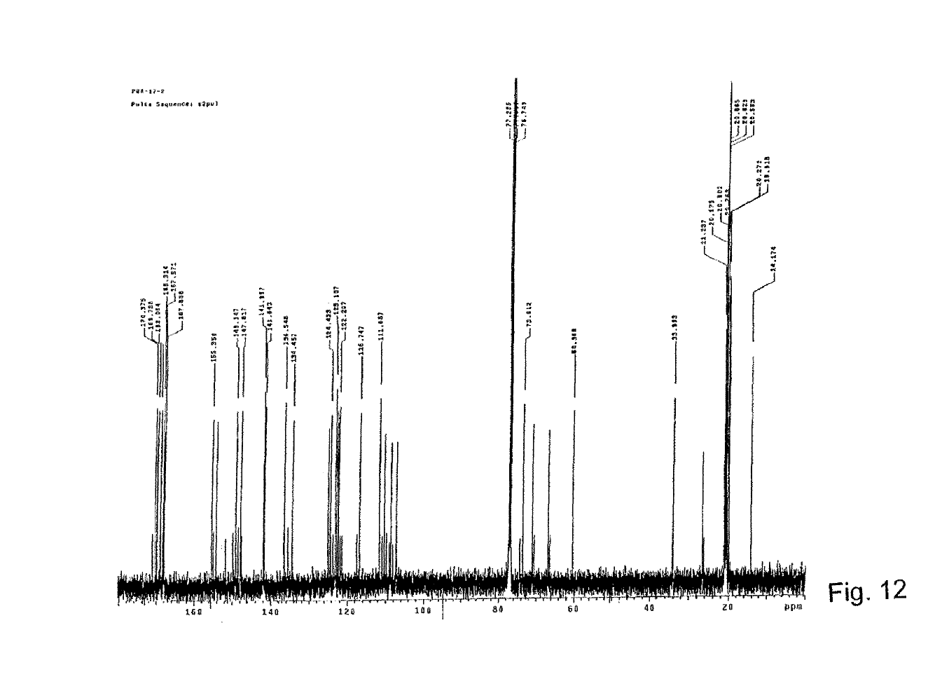

One and 2D NMR experiments (see FIGS. 11, 12)(Table 2) of the H2 peracetate showed it to be a decaacetate. Two sets of signals were seen in both the ¹H and ¹³C NMR spectra, in a ratio of three to one. These were due to rotational isomers (atropisomers), shown by opposite phase cross peaks in the nuclear Overhauser enhancement spectroscopy (NOESY) spectrum to interconvert in the time frame of the NMR experiment. It would not be possible to separate these atropisomers by chromatography. If they could be separated by crystallization, they would revert to a mixture upon taking into solution for biological assay. We solved the structure using the signals of the major atropisomer.

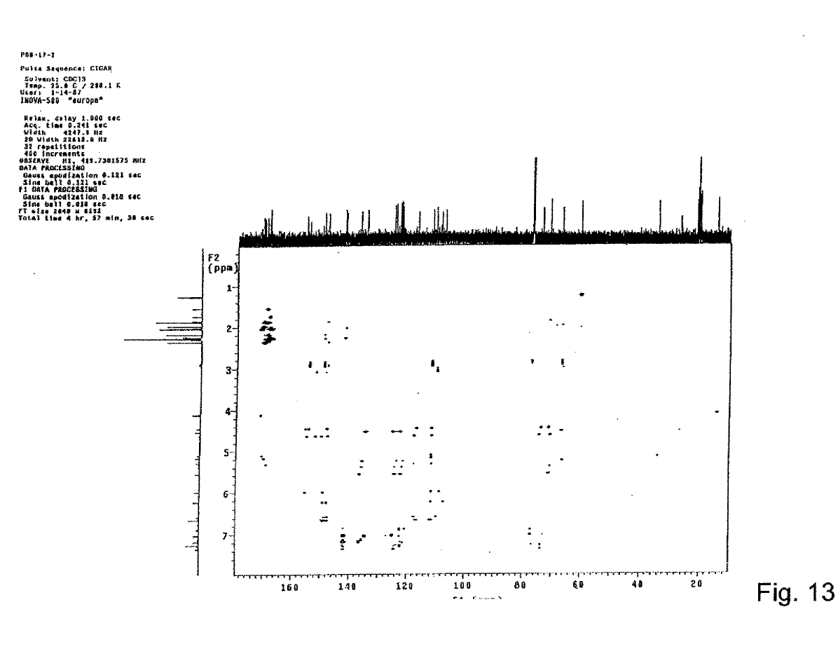

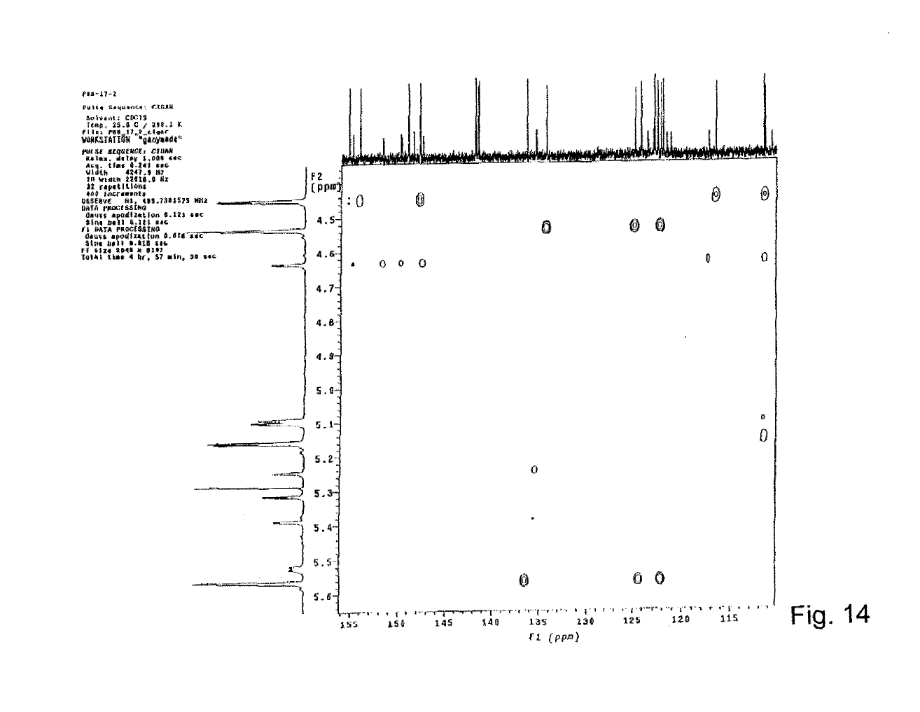

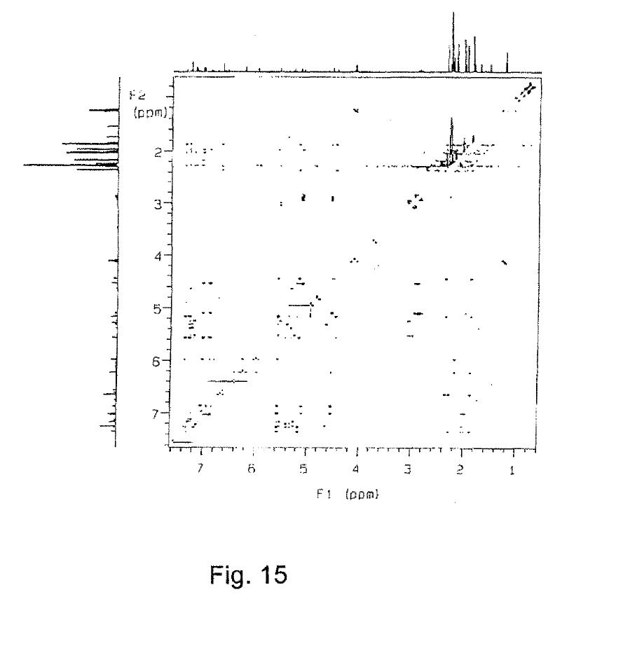

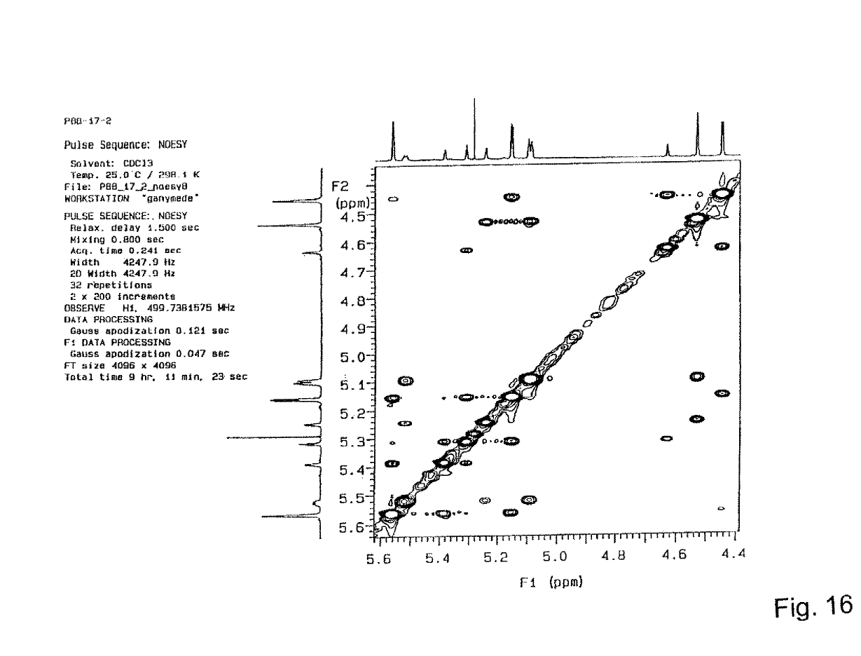

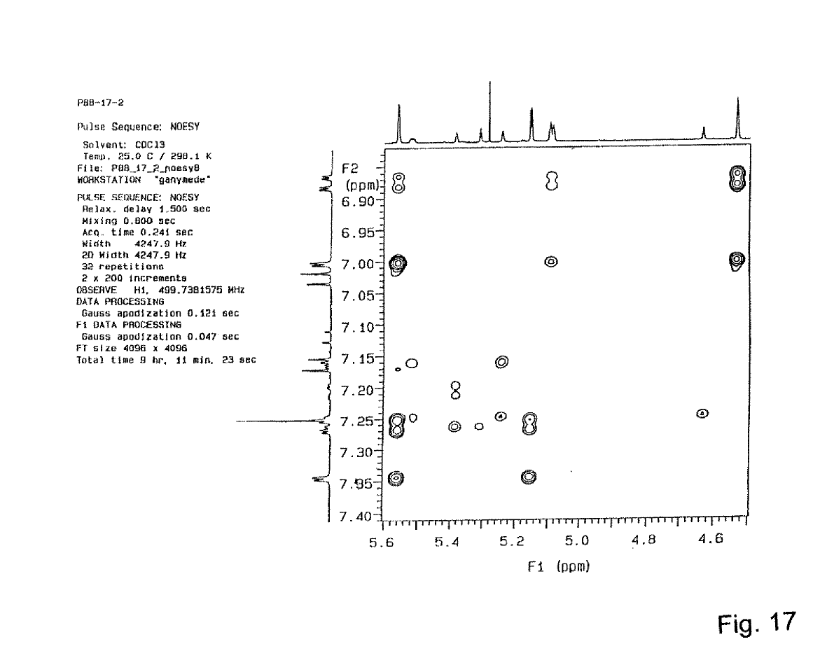

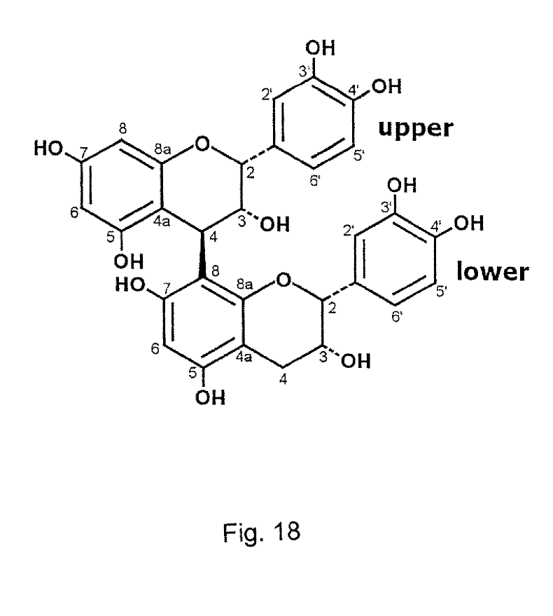

The presence of two flavan-3-ol units could be seen from the four ¹³C signals for the C-2 and C-3 positions in the 60-80 region, as well as a signal at 26.65 for the free C-4 position of the lower unit and a signal at 33.99 for the linked C-4 of the upper unit. A CIGAR ¹H-¹³C correlation experiment (FIGS. 13, 14) showed that the two units were connected from the 4(u) position to the 8(l) position, by the correlations from H-4(u) to C-8(l) and C-8a(l). The stereochemistries at C-2 and C-3 of both upper and lower units was shown to be the same as in epicatechin by the similar chemical shifts of the ¹H and ¹³C signals for the lower unit, as well as the similar low coupling constants between H-2 and H-3 in both units. The stereochemistry of the linkage was shown to be 4β→8 from the NOESY interactions (FIGS. 15-17), in particular the lack of an interaction between H-2(u) and H-4(u), and the presence of an interaction between H-2(u) and H-6′(l), and between H-3(u) and H-6′(l). The structure of the natural product H2 was therefore assigned to be epicatechin-4β→8-epicatechin FIG. 18).

Epicatechin-4β→8-epicatechin is also known as procyanidin B2 or proanthocyanidin B2. Our NMR data of H2 match partial NMR data published on procyanidin B2 (Kashiwada et al, Chem. Pharm. Bull. 38:888-893, 1990; Porter et al, J. Chem. Soc. Perkin 1:1217-1221, 1982), and our data of the H2 peracetate (FIG. 10; Table 2) exactly matched the published data on peracetylated procyanidin B2 (Franck et al, ACH Models in Chemistry 136:511-517, 1999). The optical rotation of +29.0° compared to a literature⁵ value of +25° showed the absolute stereochemistry to be the same as found previously.

TABLE 2
500 MHz NMR data of the H2 Peracetate in Deuterochloroform, Major Atropisomer.

| | C^{a} | H^{b} | CIGAR^{C} H-C | selected NOE interactions^{d} |
|---|---|---|---|---|
| lower | | | | |
| 2 | 77.00 | 4.53, br s | 3l, (4)l, (8a)l, 1′l, 2′l, 6′l | 3l, 4l, 2′l, 6′l |
| 3 | 66.81 | 5.09, br d, 5 | (4)l, 4al, 3-Oacl | 2l, 4l, 2′l, 6′l |
| 4 | 26.65 | 2.85 br d 18 | 2l, 3l, 4al, 5l, 8al | 2, 3 |
| | | 2.92 dd 18, 5 | | |
| 4a | 111.68 | | | |
| 5 | 149.07 | | | |
| 6 | 110.33 | 6.64, s | 4al, 5l, 7l, 8l | |
| 7 | 147.82 | | | |
| 8 | 116.75 | | | |
| 8a | 154.21 | | | |
| 1′ | 134.46 | | | |
| 2′ | 122.46 | 7.00, d 2 | 6′l | |
| 3′ | 141.64 | | | |
| 4′ | 141.97 | | | |
| 5′ | 122.78 | 7.02, d, 7 | 1′l, 3′l, 4′l, 6′l | |
| 6′ | 125.06 | 6.87, dd, 2, 7 | | |
| 3 OAc | 170.38 | | 1.97 s | |
| 5 OAc | 167.97 | | 5l, 2.27 s | |
| 7 OAc | 169.98 | | 7l, 2.35 s | |
| 3′/4′ OAc | 168.31 | | 2.27 s | |
| 4′/3′ OAc | 167.86 | | 2.02 s | |
| upper | | | | |
| 2 | 73.61 | 5.56, br s | 3u, (4u), 1′u, 2′u, 6′u | 3u, 2′u, 6′u, 2′l, 6′l |
| 3 | 71.06 | 5.15, dd, 1, 2 | 3-OAcu, 4u, 4au, (8l) | 2u, 4u, 2′u, 6′u |
| 4 | 33.99 | 4.45, d 2 | 2u, 3u, 4au, 5u, 7l, 8l, 8au, 8al | 3u, 2′u, 6′u, 2′l, 6′l |
| 4a | 111.57 | | | |
| 5 | 147.91 | | | |
| 6 | 108.63 | 6.22, d J 1 Hz | 4au, 5u, 7u, 8u | |
| 7 | 149.15 | | | |
| 8 | 107.23 | 5.98, d, J 1 Hz | 4au, 6u, 7u, 8au | |
| 8a | 155.35 | | | |
| 1′ | 136.55 | | | |
| 2′ | 122.21 | 7.35, d, 2 | 6′u | |
| 3′ | 141.75 | | | |
| 4′ | 141.99 | | | |
| 5′ | 123.11 | 7.16, d, 7 | 1′u, 3′u, 4′u | |
| 6′ | 124.43 | 7.26, dm, J 7 Hz | | |
| 3 OAc | 169.74 | | 1.86 s | |
| 5 OAc | 169.06 | | 5u, 1.86 s | |
| 7 OAc | 168.87 | | 7u, 2.17 s | |
| 3′/4′OAc | 168.31 | | 2.27 s | |
| 4′/3′OAc | 167.87 | | 2.02 s | |

^{a}shift in ppm.
^{b}Shift in ppm, multiplicity, couplings in Hz.
^{c}Brackets indicate weak correlations, l = lower unit, u = upper unit.
^{d}Rocorded for H-2, H-3 and H-4 only. OAc methyl groups not shown

### Example 3

### Isolation and Identification of Peak H1 from PTI-777

### General Experimental Procedures

The minor component of peak H, referred to as H1, of the PTI-777 extract was also isolated by a series of chromatographic techniques, monitored by HPLC (see Example 17 Experimental Procedures for details). We initially separated the original PTI-777 extract (see HPLC tracing, FIG. 2) by column chromatography over silica gel, where 20% methanol in chloroform gave a fraction rich in the two components of peak H (134 mg). An HPLC method was developed to separate the two main components of peak H on a preparative scale)(see HPLC tracing, FIG. 3), to give us a mostly pure H1(16 mg)(see HPLC tracing, FIG. 4) and pure H2 (23 mg).

A ⁻ve ion electrospray mass spectrum of H1 gave a clean 100% ion at 577 daltons. This is appropriate for the molecular ion (M⁺−H) of a molecular formula of C₃₀H₂₆O₁₂ (molecular weight 578), such as a dimer of two epicatechin, or isomeric units. We had previously isolated and identified epicatechin from the PTI-777 extract.

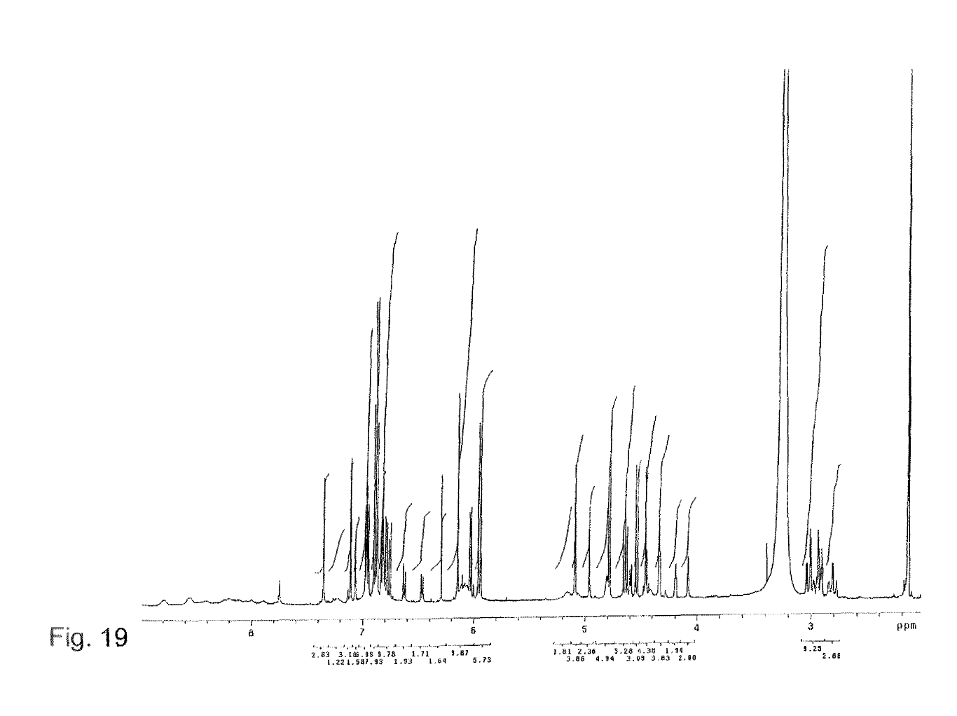

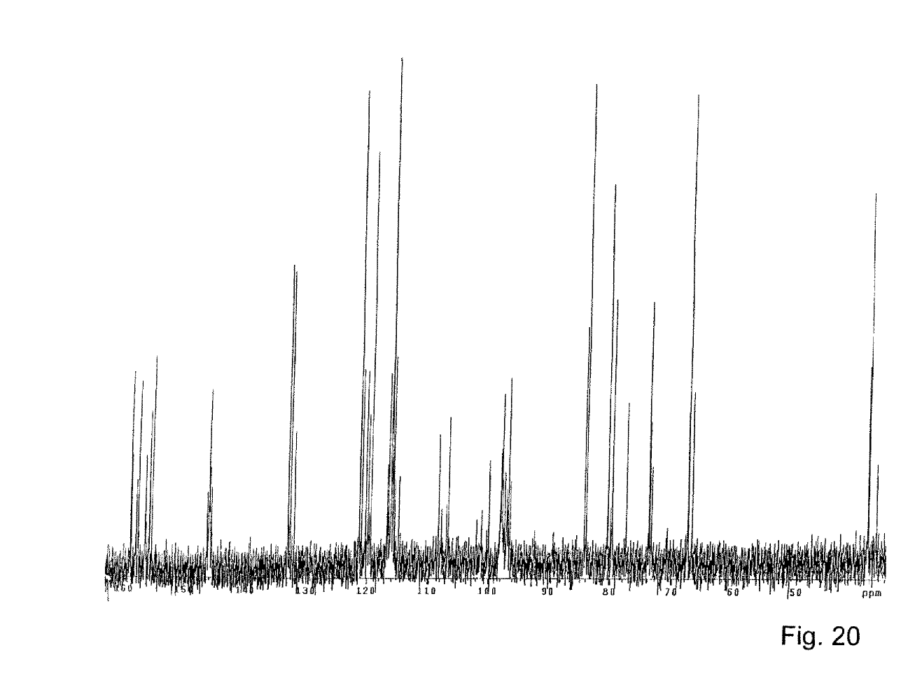

A ¹H NMR spectrum (FIG. 19) and ¹³C NMR spectrum FIG. 20) of peak H1 showed two sets of signals, consistent with some kind of flavonol dimer, with major and minor atropisomers present.

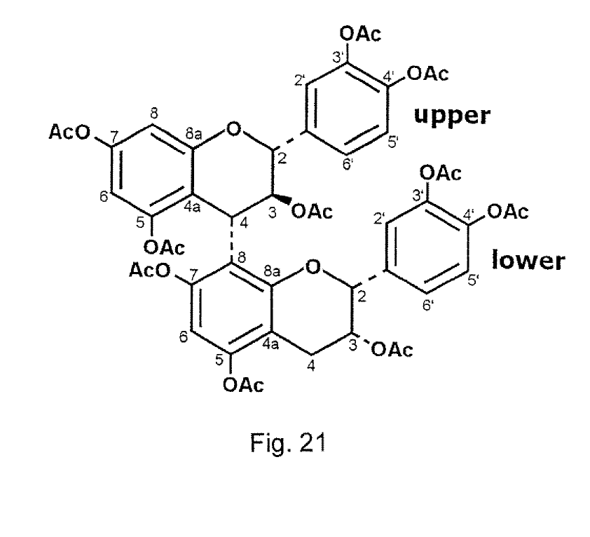

Acetylation of a sample of H1 gave a peracetate (FIG. 21), which was purified by column chromatography over silica gel. A larger sample of this peracetate, identical by NMR and TLC, was also obtained by silica gel separation of the two main products from acetylation of a fraction rich in H1 and H2.

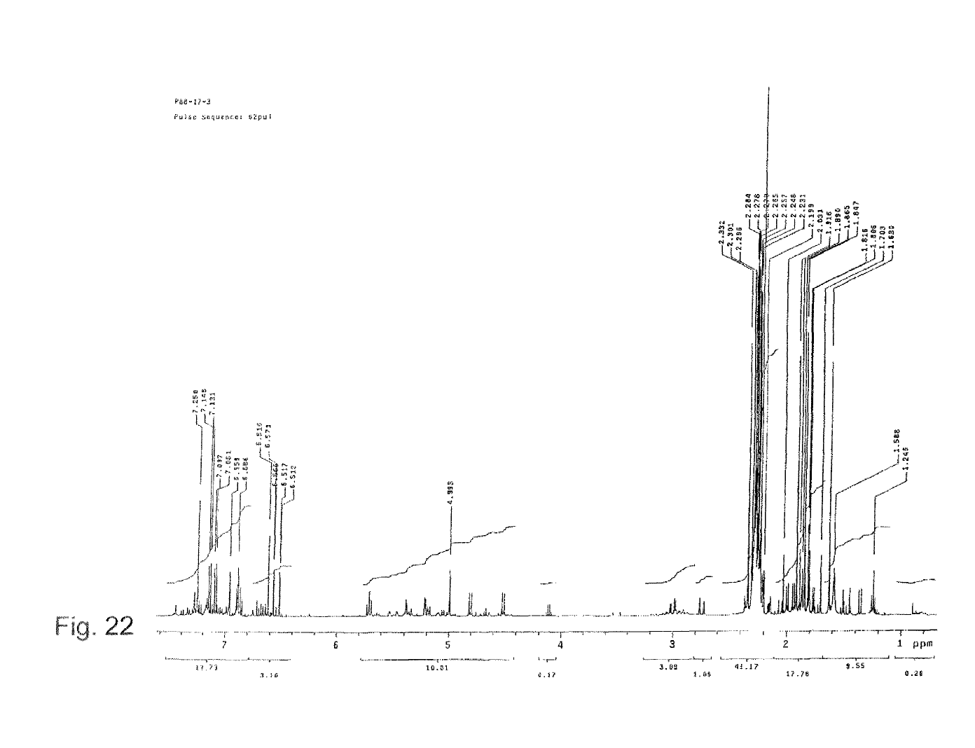

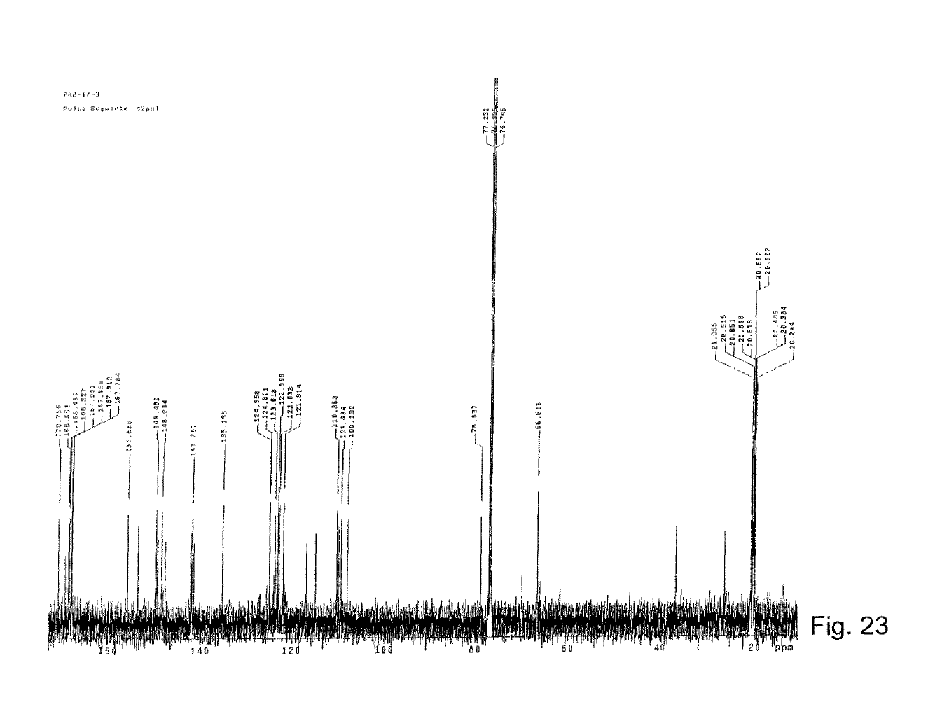

One and 2D NMR experiments (FIGS. 22, 23) (Table 3) on the H1 peracetate showed it to be a decaacetate. The structure was solved using the signals of the dominant atropisomer. The presence of two flavan-3-ol units could be seen from the four ¹³C signals in the 60-80 region (FIG. 23), as well as a signal at 26.56 for the free C-4 position of the lower unit and a signal at 36.72 for the coupled C-4 of the upper unit (FIG. 23). The positions of the ¹³C signals and the small couplings of the ¹H signals of the lower unit were typical of an epicatechin unit, whilst the positions of the ¹³C signals and the much larger couplings of the ¹H signals of the upper unit were typical of a coupled catechin (Fletcher et al, JCS Perkin 1:1628-1637, 1977).

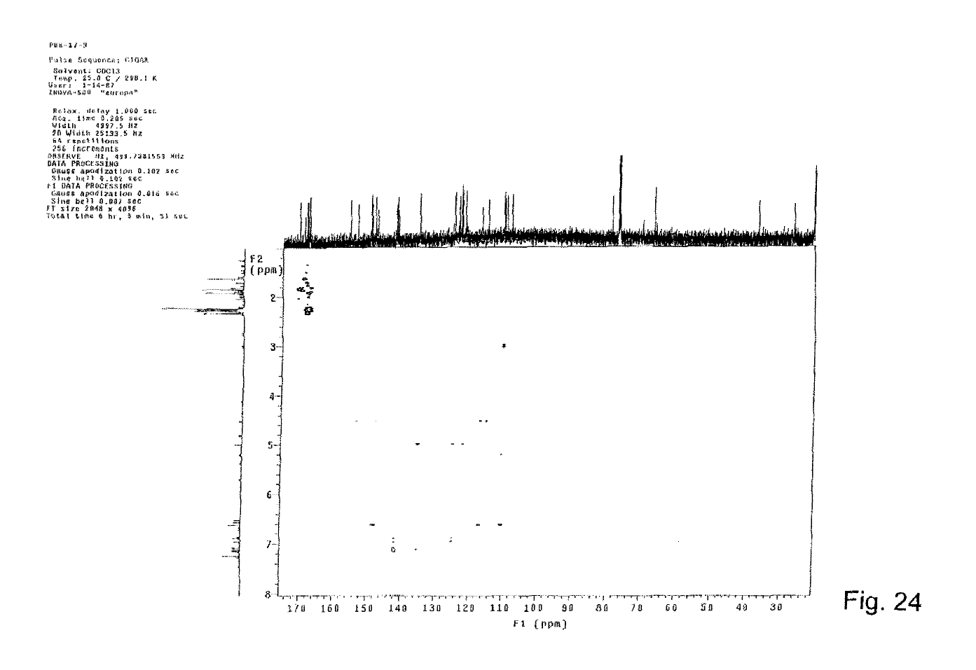

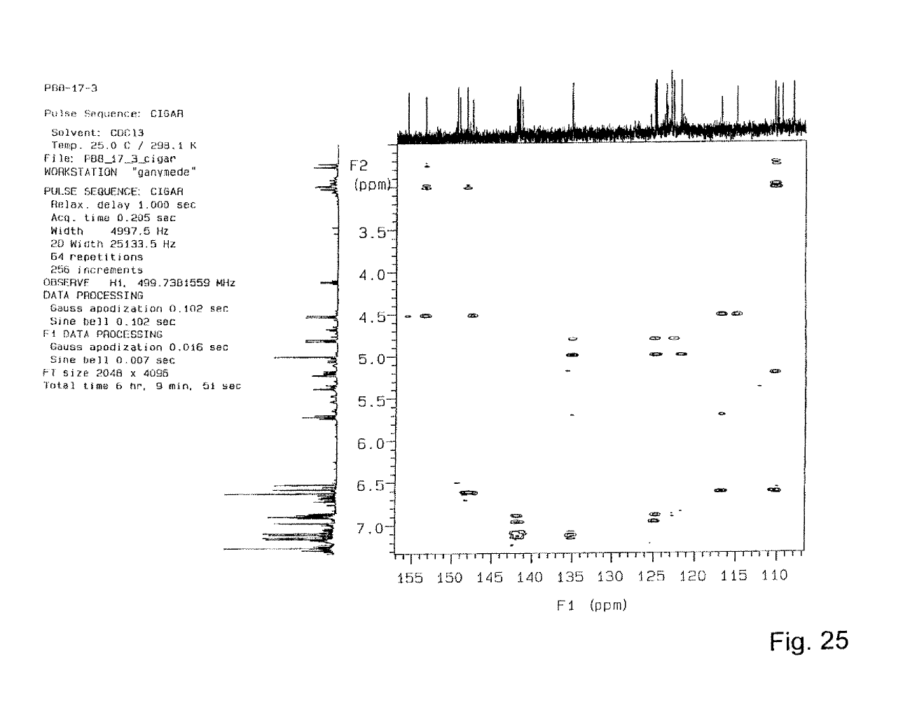

A CIGAR ¹H-¹³C correlation experiment (FIGS. 24, 25) showed that the two units were connected from the 4(u) position to the 8(l) position, by the correlations from H4(u) to C-8(l) and C-8a(l).

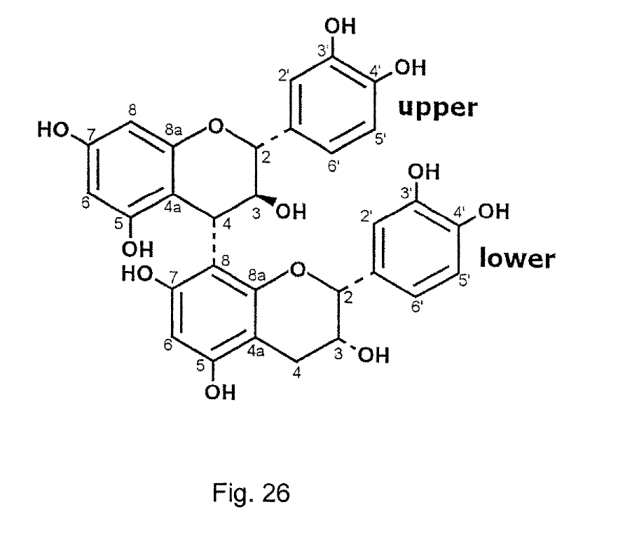

The structure of the natural product H1 was therefore determined to be catechin-4α→8-epicatechin, also known as procyanidin B4 or proanthocyanidin B4 (FIG. 26). Our NMR data on compound H1 matched partial NMR data published on procyanidin B4 (Thompson et al, JCS Perkin 1: 1387-1399, 1972; Fletcher et al., JCS Perkin 1:1628-1637, 1977) and our data of acetylated compound H1 (FIG. 21; Table 3) matched partial NMR data published on peracetylated procyanidin B4 (Thompson et al, JCS Perkin 1:387-1399, 1972: Fletcher et al, JCS Perkin 1: 1628-1637, 1977). The optical rotation of −102° (MeOH) for compound H1 compared to a literature value of −193′ (EtOH)(Thompson et al, JCS Perkin 1:1387-1399, 1972) showed the absolute stereochemistry to be the same as found previously.

### Peak H1 Data Summary

Aliquots (14×70 μl) of fractions 20-24 (134 mg in 1 ml)(FIG. 3) from the silica gel column were separated by HPLC using method 2 (as described in Example 1). The peaks between 14.5 and 16.2 and between 16.2 and 19.0 minutes were collected, then freeze dried to give two products, about 80% pure H1, retention time 15.1 Method 2 (16 mg)(FIG. 4) as a white solid; and pure H2 (23 mg) as a white solid.

−ve electrospray m.s. 577 (M⁺−H, 100%)

molecular weight 578

¹H NMR ((CD)₃)₂CO)(major isomer, partial data): 2.92 (1H, dd, 2, 16, H-4l), 3.02 (1H, dd, 5, 16, H-4l), 4.34 (1H, bs, H-3l), 4054 (1H, d, 10, H-4-u) 4.64 (1H, dd, 8, 10, H-3u), 4.79 (1H, d, 8, H-2u), 5.09 (1H, s, H-2l), 5.94 (1H, d, 2, H-6u), 5.96 (1H, d, 2, H-8u) and 6.15 (1H, s, H-6l).

¹³C NMR ((CD₃)₂CO)(major isomer, partial data):30.30 (determined from HSQC correlation), 38.85, 67.49, 73.89, 80.52, 83.99, 96.76, 97.81, 98.02, 106.66, 108.29 and 116.11.

UV (MeOH) λ max (log ε) 209 (4.71), 225sh (4.44) and 281 (3.65) nm;

[∝]²⁴₅₈₉ₙₘ−103.0°, [∝]²⁴₅₇₇ₙₘ−118.30°, [∝]²⁴₅₄₆ₙₘ−153.1°, [∝]²⁴₄₃₅ₙₘ−379.9°, [∝]²⁴₄₀₅ₙₘ−469.1° (c 0.2, MeOH)

### Acetylation of H1 Protocol

A sample of a fraction rich in peaks H1 and H2, from a second silica gel column of PTI-777 (50 mg) was dissolved in a mixture of acetic anhydride (0.5 ml) and pyridine (0.5 ml). The mixture stood at room temperature for 18 hours, then the solvents were removed in vacuo. Purification by column chromatography over silica gel, eluting with 20% ethyl acetate in dichloromethane gave the H2 peracetate (38 mg) followed by the H1 peracetate 5 (15 mg) as a colorless gum. The NMR data is shown in Table 3 below.

TABLE 3
500 MHz NMR data of the H1 peracetate in deuterochloroform,
major atropisomer.

| | | | CIGAR^{c} |
|---|---|---|---|
| | C^{a} | H^{b} | H-C |
| lower | | | |
| 2 | 77.10 | 5.00, br s | 4l |
| 3 | 66.62 | 5.21, br d, J 5 Hz | 4l, 2l |
| 4 | 26.56 | 2.75 br d J 18 Hz | 2l, 3l, 4al, 5l, 8al |
| | | 3.00 dd J 18, 5 Hz | as above |
| 4a | 110.36 | | |
| 5 | 148.29 | | |
| 6 | 109.49 | 6.62, s | 4al, 5l, 7l, 8l |
| 7 | 147.57 | | |
| 8 | 116.86 | | |
| 8a | 153.46 | | |
| 1′ | 135.16\hat{ } | | |
| 2′ | 121.81 | 6.90, d J 2 Hz | not defined |
| 3′ | 142.10* | | |
| 4′ | 141.96* | | |
| 5′ | 123.00\hat{ } | 7.14, d, J 7 Hz | 1′l, 3′l, 4′l, 6′l |
| 6′ | 124.82* | 6.86, dd, 2, 7 Hz | not defined |
| upper | | | |
| 2 | 78.99 | 4.81 d 8 | 4u |
| 3 | 70 | 5.70 t 8 | 2u, 4u |
| 4 | 36.72 | 4.52 d 8 | 8au, 8al, 7l, 8l, 4au |
| 4a | 115.01 | | |
| 5 | 149.48* | | |
| 6 | 108.13 | 6.57 d 1 | not defined |
| 7 | 149.20* | | |
| 8 | 110.06 | 6.52 d 1 | not defined |
| 8a | 155.69 | | |
| 1′ | 135.21\hat{ } | | |
| 2′ | 122.7 | 6.96 1 | not defined |
| 3′ | 141.71* | | |
| 3′ | 141.41* | | |
| 5′ | 123.62* | 7.09 d 7 | |
| 6′ | 124.96 | 6.89 m | 2u, 2′u |

^{a}shift in ppm.
^{b}Shift in ppm, multiplicity, couplings in Hz.
l = lower unit,
u = upper unit.
OAc groups not shown.

### Example 4

### Isolation and Identification of Peak K2 from PTI-777

### General Experimental Procedures

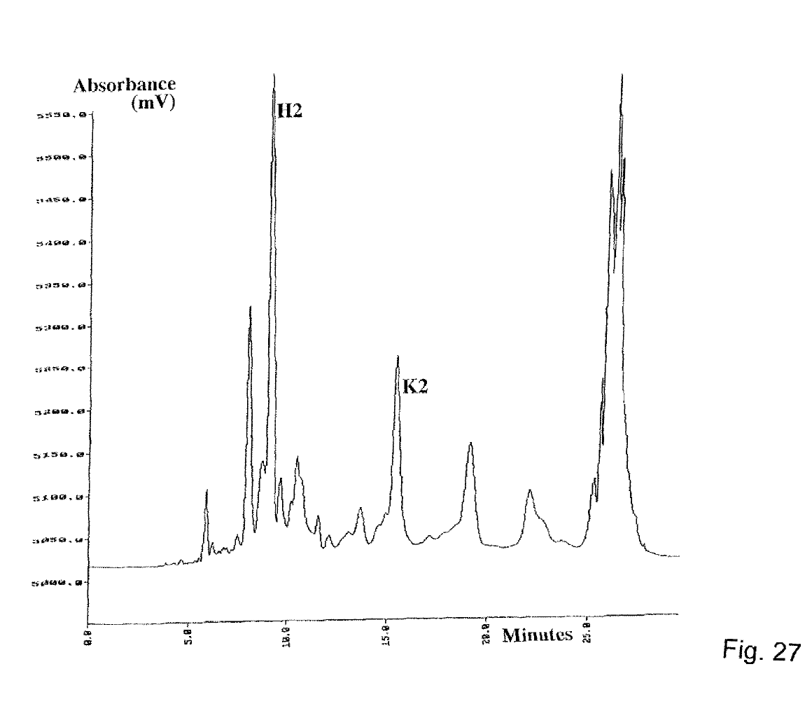

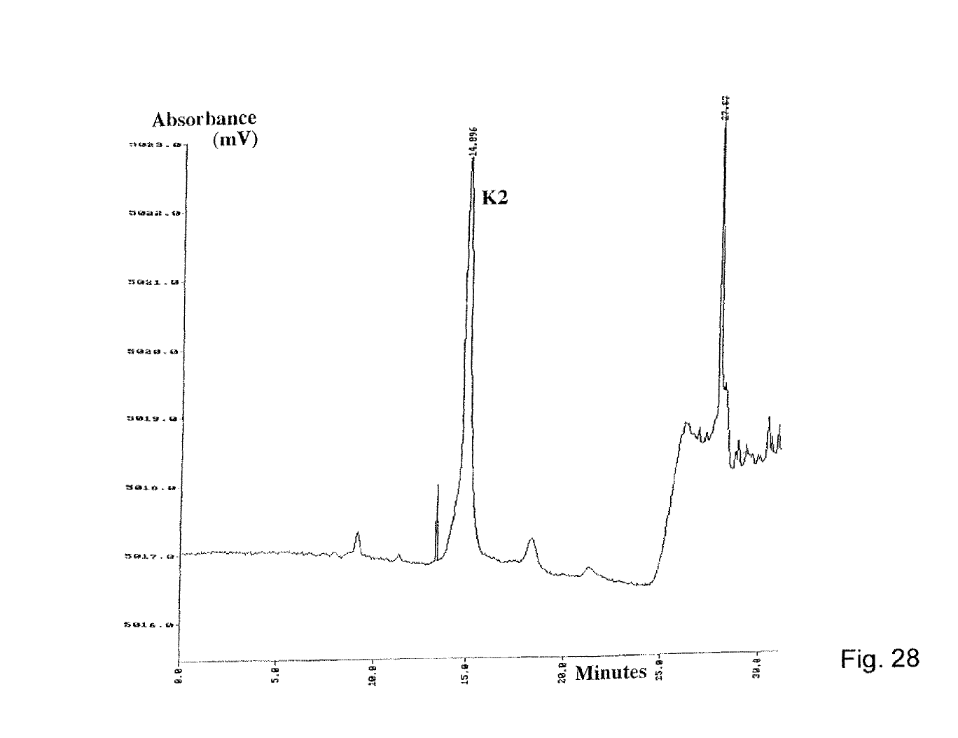

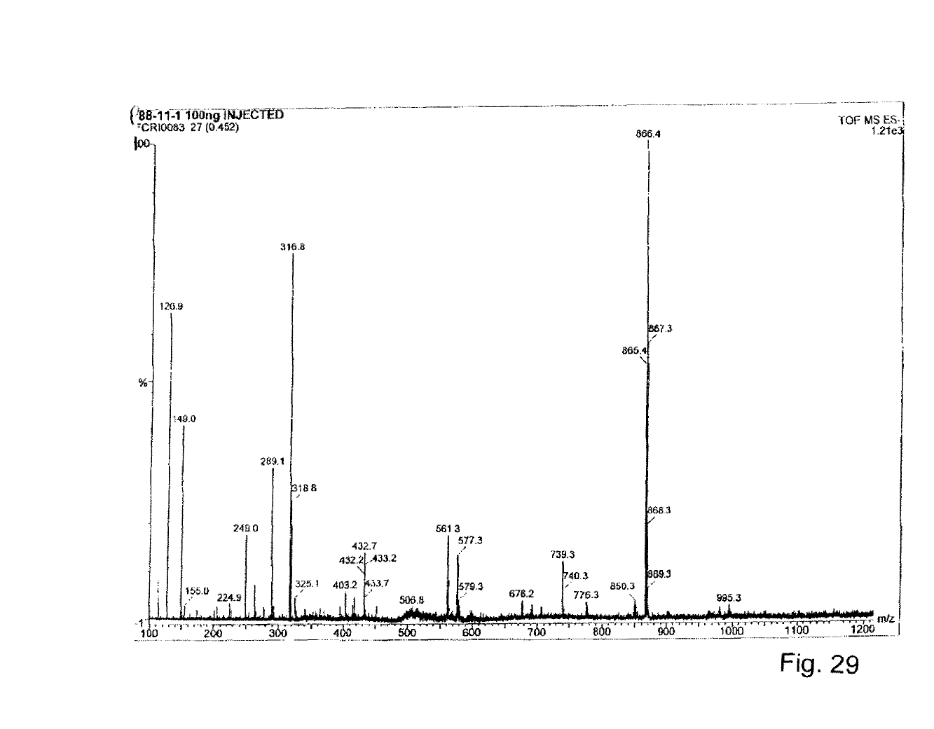

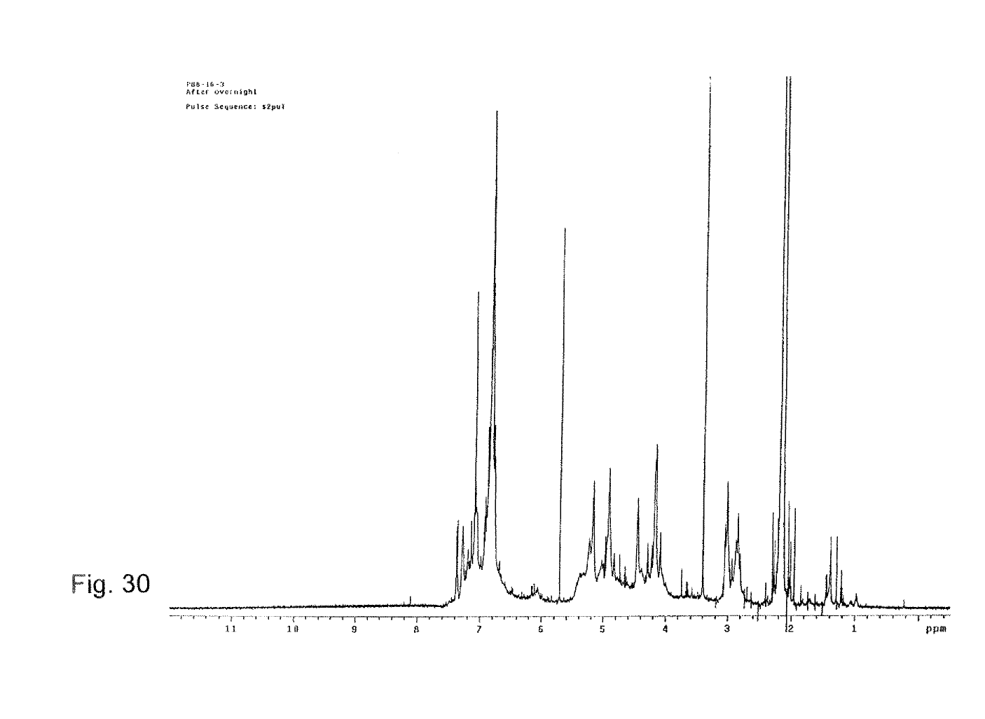

The major component of peak K, called K2, of the PTI-777 extract was also isolated by a series of chromatographic techniques, monitored by HPLC (see Example 2, Experimental Procedures for details). We initially separated the original PT-777 extract by column chromatography over silica gel, when 40% methanol in chloroform gave a fraction rich in the major component of peak K (Table 1). Preparative HPLC on a fraction rich in K2 (FIG. 27), using method 1 (see example 1) gave a pure sample of peak K2 (FIG. 28). A −ve ion electrospray mass spectroscopy of this showed it to have a molecular ion M⁺ of 866 (FIG. 29). This is appropriate for a molecular formula of C₄₅H₃₈O₁₈ (molecular weight=866), such as a trimer of three epicatechin or catechin units. The initial ¹H NMR (FIG. 30) showed there to be similar broad peaks to that seen in H2, so it was decided to acetylate the compound to definitely identify the structure of K2.

A further fraction from the silica gel column which was rich in peak K2 was acetylated as before (in Examples 1 and 2) to enable us to obtain more material for structure elucidation. The peracetate of K2 was purified by column chromatography over silica gel.

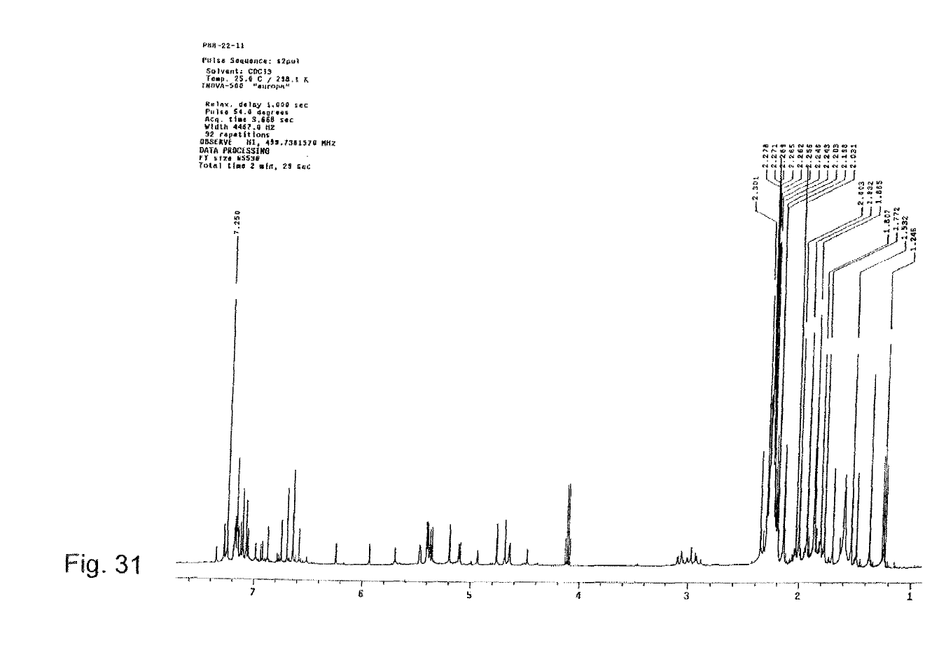

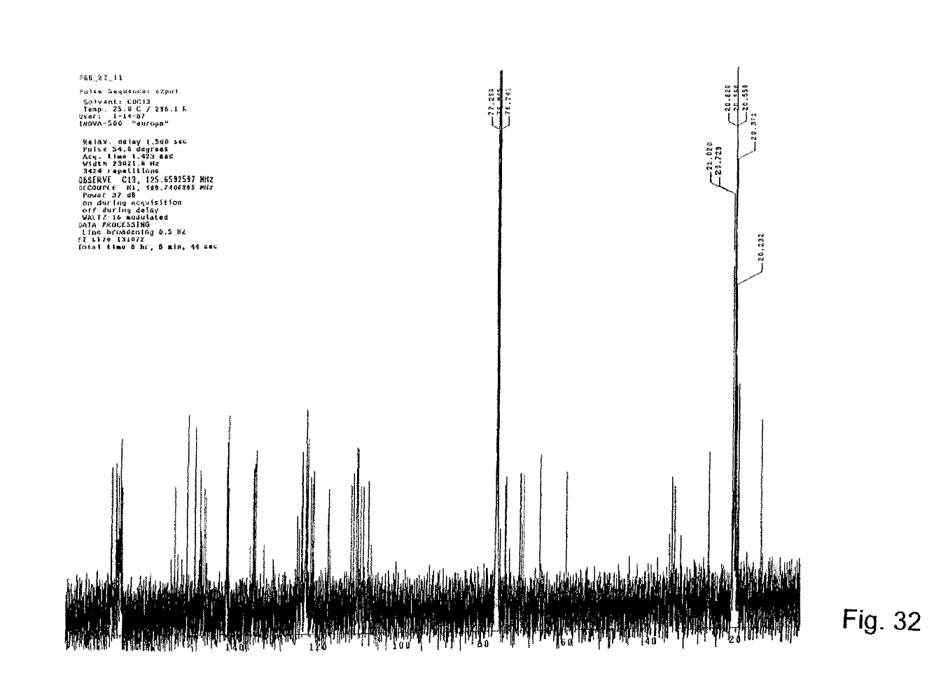

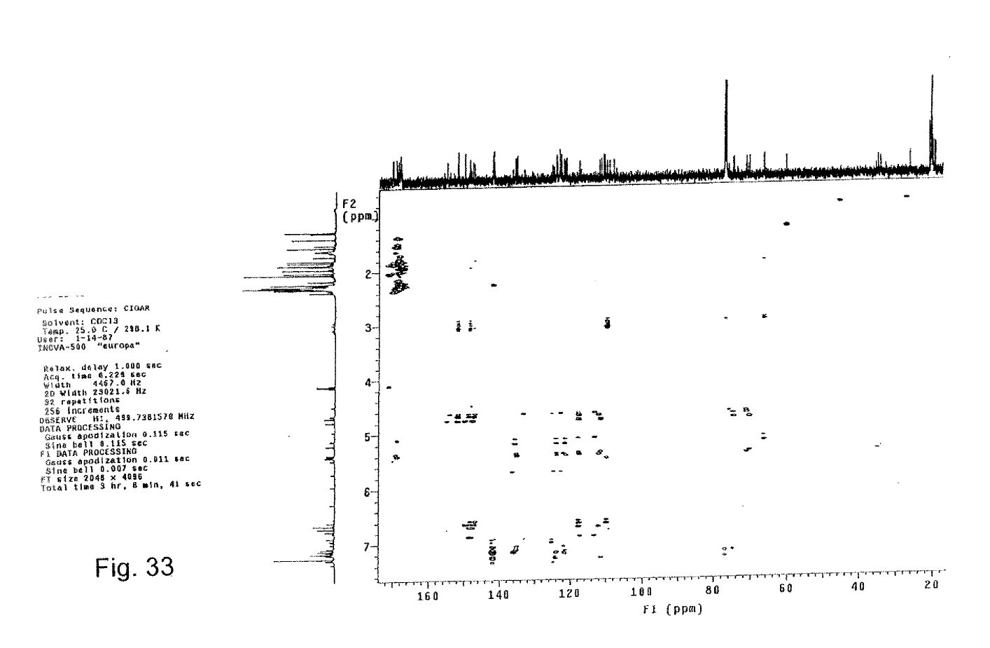

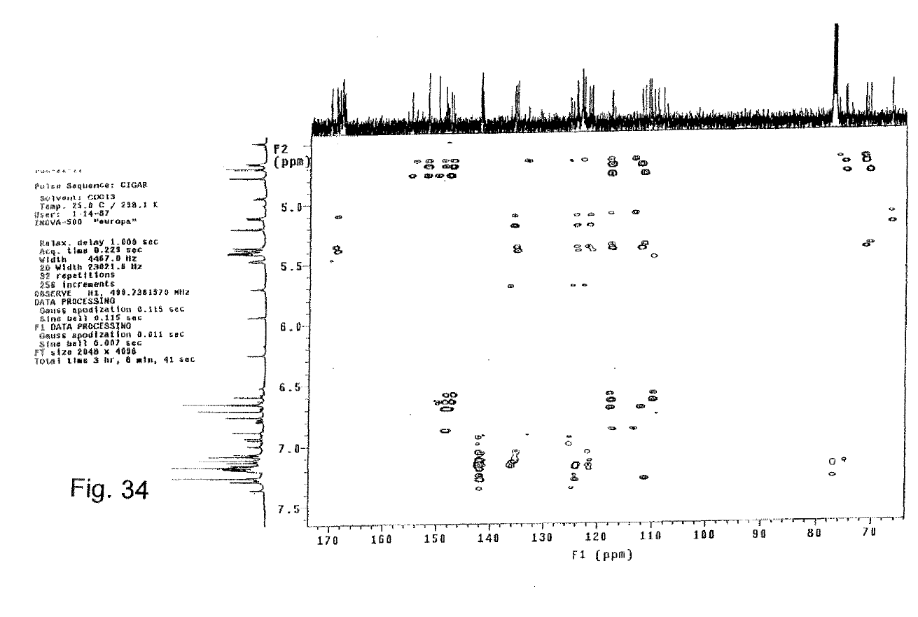

One (FIGS. 31, 32) and 2D NMR experiments FIGS. 33, 34) were carried out on the K2 peracetate. Two sets of signals were seen in both the ¹H and ¹³C NMR spectra, in a ratio of three to one. These were due to optional isomers (atropisomers) as discussed in Example 2. We solved the structure using the signals of the major isomer (see Table 4 below).

The positions of the ¹³C signals and the small couplings of the ¹H signals of the lower unit was typical of epicatechin, and the positions of the ¹³C signals and the small couplings of the ¹H signals of the other two units were typical of coupled epicatechin Fletcher et al, J.C.S. Perkin 1:1628-1637, 1977). The presence of three flavan-3-ol units could be seen from the six ¹³C signals in the 60-80 region, as well as a signal at 26.39 for the free Cut position of the lower unit and signals at 34.36 and 35.04 for the coupled C-4's of the other units.

A CIGAR ¹H-¹³C con-elation experiment (FIGS. 33, 34) showed that the two units were connected from the 4 (upper) positions to the 8 (lower) positions, by the correlations from H-4(u) to C-8(m) and C-8a(m), and from H4(m) to C-8(l) and C-8a(l).

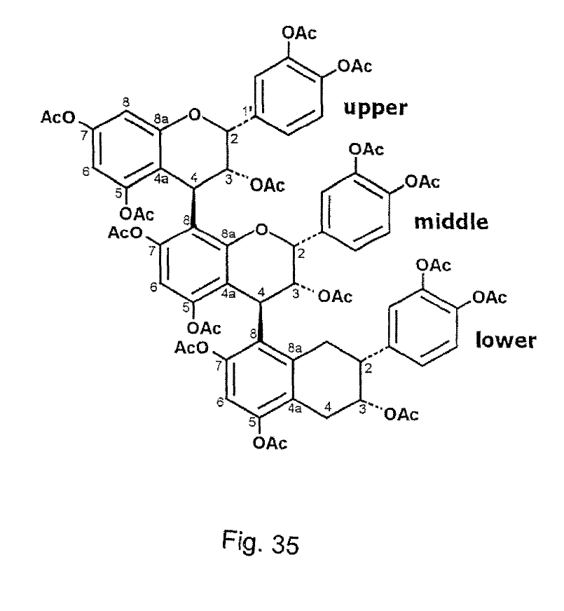

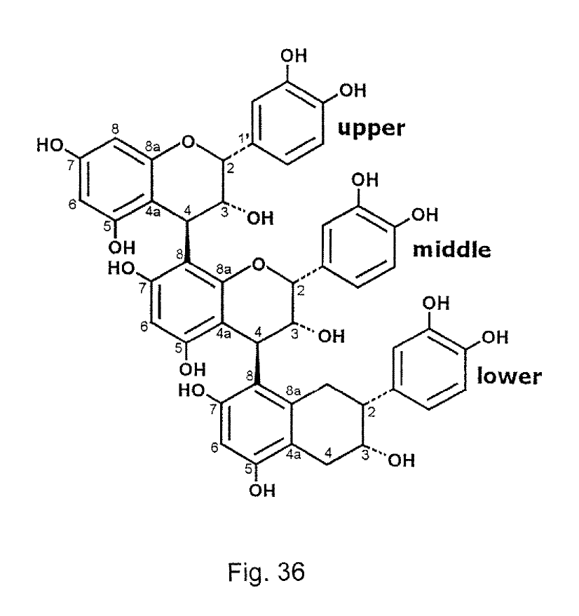

K2 peracetate was therefore determined to be the structure shown in FIG. 35. Our NMR data on the K2 peracetate shown in FIG. 35 matched partial NMR data published on procyanidin C1 (Porter et al, J.C.S. Perkin 1:1217-1221, 1982; Hemingway et al, J.C.S. Perkin 1:1209-1216, 1982), but we could not find any ¹³C NMR data published on structure K2 (FIG. 36). The optical rotation of +60.9° (MeOH) for structure K2 compared to the literature value of +92° (H₂O) showed it to have the same absolute stereochemistry as that published. K2 was therefore identified as epicatechin-4β→8-epicatechin-4β→8-epicatechin or procyanidin C1 (FIG. 36).

### Peak K2 Data Summary

Aliquots (8×70 μl) of fractions 3540 (226 mg in 1.0 ml) from the above silica gel column were separated by HPLC using method 1 (as described in Example 2). The peak between 12.90 and 15.70 minutes was collected. P88-21-2, retention time 15.1 minutes (5 mg)(FIGS. 27, 28) which is peak K2.

¹³C NMR ((CD₃)₂CO)(partial data on major isomer): 29.3 (determined from HSQC correlation), 37.6, 37.6, 67.10, 72.68, 73.69, 77.48, 77.61, 79.85.

UV (MeOH) λ max (log ε) 211 (4.95), 226sh (4.66) and 281 (3.94) nm;

[α]²⁴₅₈₉ₙₘ+60.9°, [α]²⁴₅₇₇ₙₘ+53.2°, [α]²⁴₅₄₆ₙₘ+40.0° (c 0.2, MeOH)

### Acetylation of K2 Protocol

A sample of K2 (5 mg) was dissolved in a mixture of acetic anhydride (0.5 ml) and pyridine (0.5 ml). The mixture was kept at room temperature for 18 hours, then the solvents were removed in vacuo. Purification by column chromatography over silica gel, eluting with 20% ethyl acetate in dichloromethane gave the K2 peracetate (2 mg) as a colorless gum.

A fraction rich in K2 (34 mg)(FIG. 28) was dissolved in a mixture of acetic anhydride (0.5 ml) and pyridine (0.5 ml). The mixture was kept at room temperature for 18 hours, then the solvents were removed in vacuo. Purification by column chromatography over silica gel, eluting with 20% ethyl acetate in dichloromethane gave the K2 peracetate (15 mg) as a colorless gum. NMR data is shown in Table 4 below.

TABLE 4
500 MHz NMR data of the K2 peracetate in deuterochloroform,
major atropisomer . . .

| | C | H | CIGAR |
|---|---|---|---|
| upper | | | |
| 2 | 74.70 | 5.37 bs | 1′u, 3u |
| 3 | 70.47 | 5.35 bs | 3-OAcu, 4au, 8m |
| 4 | 34.36 | 4.76 s | 2u, 3u, 4au, 5u, 7m, 8m, |
| | | | 8au, 8am. |
| 4a | 111.61 | | |
| 5 | 149.88 | | |
| 6 | 108.12 | 6.75 d 2 | |
| 7 | | | |
| 8 | 109.30 | 6.64 d 2 | |
| 8a | 154.88 | | |
| 1′ | 135.44 | | |
| middle | | | |
| 2 | 74.94 | 5.40 bs | 1′m |
| 3 | 71.29 | 5.38 bs | 3-OAcm, 4am, 8l |
| 4 | 35.04 | 4.69 s | 2m, 3m, 4am, 5m, 7l, 8l, 8al |
| 4a | 112.17 | | |
| 5 | 148.58 | | |
| 6 | 110.95 | 6.70 s | 4am, 5m, 7m, 8m |
| 7 | 147.59 | | |
| 8 | 117.72 | | |
| 8a | 151.87 | | |
| 1′ | 135.15 | | |
| lower | | | |
| 2 | 77.00 | 5.19 s | 1′l, 3l |
| 3 | 66.57 | 5.69 m | 4al |
| 4 | 26.39 | 2.98 m | 4al, 5l, 8al |
| 4a | 109.96 | | |
| 5 | 148.49 | | |
| 6 | 110.63 | 6.63 s | 4al, 5l, 7l, 8l |
| 7 | 147.18 | | |
| 8 | 117.60 | | |
| 8a | 151.73 | | |
| 1′ | 135.72 | | |

^{a}shift in ppm.
^{b}Shift in ppm, multiplicity, couplings in Hz.
l = lower unit,
m = middle unit,
u = upper unit. Ring C and OAc groups not shown.

### Example 5

### Efficacy of Proanthocyanidins H2, H1 and K2 as Disruptors of Aβ Fibrils

In the next set of studies, we demonstrate that pure compounds H2 (epicatechin-4β→8-epicatechin), H1 (catechin-4α→8-epicatechin) and K2 (epicatechin-4β→8-epicatechin-4β→8-epicatechin) isolated as described in Examples 1-3, were tested for efficacy in different to amyloid and α-synuclein/NAC diseases. In a first set of studies, the efficacy of these proanthocyanidin pure compounds were tested for their ability to cause a potent disassembly/disruption of pre-formed amyloid fibrils of Alzheimer's disease (i.e. consisting of Aβ 1-42 fibrils).

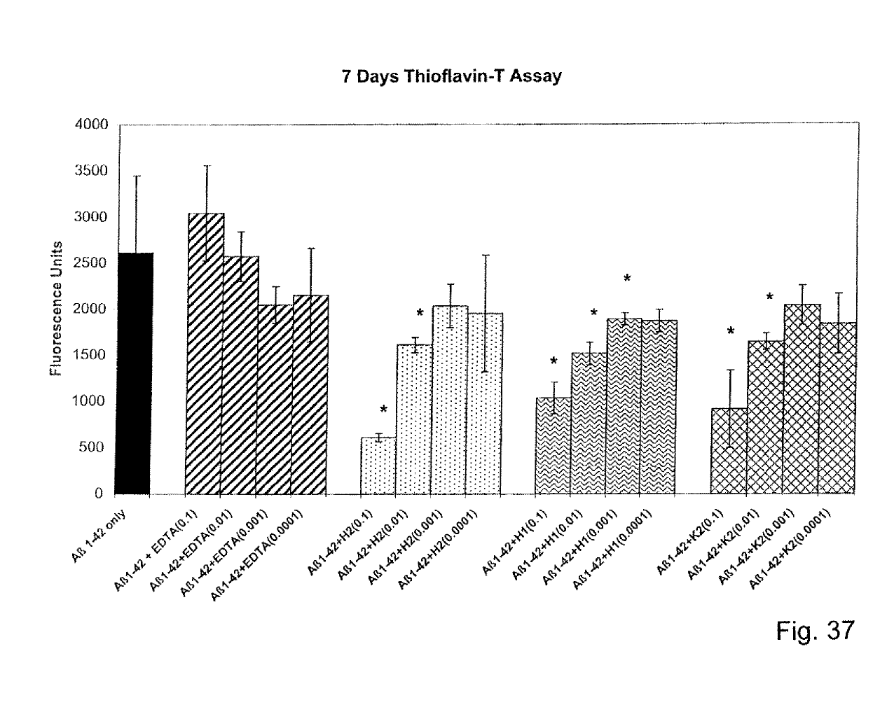

In one study, Thioflavin T fluorometry was used to determine the effects of H2, H1, K2 and EDTA (as a negative control) on disassembly/dissolution of pre-formed Aβ 1-42 fibrils (FIG. 37). In this assay Thioflavin T binds specifically to fibrillar amyloid, and this binding produces a fluorescence enhancement at 485 nm that is directly proportional to the amount of amyloid fibrils formed. The higher the fluorescence the greater the amount of amyloid fibrils formed (Naki et al. Lab. Invest. 65:104-110, 1991; Levine III, Protein Sc. 2:404-410, 1993; Amyloid Int. J. Exp. Clin. Invest. 2:1-6, 1995).

In this study, 25 μM of pre-fibrillized Aβ 1-42 (Bachem Inc) was incubated at 37° C. for 1 week either alone, or in the presence of EDTA, H2, H1, or K2 at an Aβ:test compound weight ratios of 1:0.1, 1:0.01, 1:0.001 or 1:0.0001. Following 3-days or 7-days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer)(pH 6.8). The fluorescence was read at 485 nm (444 nm excitation wavelength) using an ELISA plate fluorometer after subtraction with buffer alone or compound alone, as blank.

The results of day 7 incubations are presented here, but similar results were obtained as early as 3 days. As shown in FIG. 37, whereas EDTA caused no significant inhibition of Aβ 1-42 fibrils at all concentrations tested, compound H2 caused a dose-dependent disruption/disassembly of preformed Aβ 1-42 fibrils, with a significant (p<0.01) 29+/−4% disruption when used at an Aβ:H2 wt/wt ratio of 1:0.01, and a significant (p<0.01) 73+/−2% disruption when used at an Aβ:H2 wt/wt ratio of 1:0.1 (i.e. 1:1 molar ratio). Similarly, compound H1 caused a dose-dependent disruption/disassembly of preformed Aβ 1-42 fibrils, with a significant 16+/−3% disruption when used at an Aβ:H1 wt/wt ratio of 1:0.001; a significant 33+/−6% disruption when used at an Aβ:H1 wt/wt ratio of 1:0.01; and a significant 54+/−8% disruption when used at an Aβ:H1 wt/wt ratio of 1:0.1 (i.e. 1:1 molar ratio). Compound K2 also caused a dose-dependent disruption/disassembly of preformed Aβ 1-42 fibrils, with a significant 27+/−4% disruption when used at an Aβ:K2 wt/wt ratio of 1:0.01; and a significant 60+/−19% disruption when used at an Aβ:K2 wt/wt ratio of 1:0.1 (i.e. 1:1 molar ratio). This study indicated that the proanthocyanidins H2, H1 and K2 were potent disruptors of Alzheimer's disease type Aβ fibrils, and exerted their effect in a dose-dependent manner.

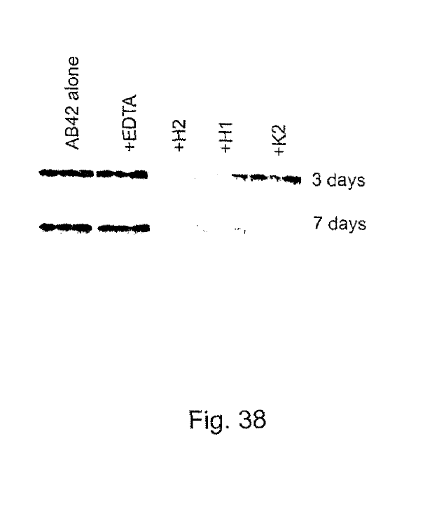

The disruption of Aβ 1-42, even in its monomeric form, was confirmed by a study involving the use of SDS-PAGE and Western blotting methods. In this latter study, triplicate samples of pre-fibrillized Aβ 1-42 (25 μM) was incubated at 37° C. for 3 and 7 days either alone or in the presence of compound H2, H1, K2 or EDTA (as a negative control). 5 μg of each sample was then filtered through a 0.2 μm filter. Protein recovered from the filtrate was then loaded, and ran on a 10-20% Tris-Tricine SDS-PAGE, blotted to nitrocellulose and detected by ECL using an Aβ-antibody (clone 6E10, Senetek). As shown in FIG. 38. Aβ 1-42 was detected as a ˜4 kilodalton band (i.e. monomeric A) following incubation alone, or in the presence of EDTA, at both 3 and 7 days. Aβ 1-42 monomers were not detected following incubation of Aβ 1-42 with either H2, H1 or K2 by 7 days of co-incubation (FIG. 38) suggesting that these compounds were capable of causing a disappearance of monomeric Aβ 1-42. Disappearance of Aβ 1-42 was already apparent even following 3 days of incubation with H2 and H1, whereas K2 took longer (i.e. 7 days) to have a full effect. This study confirmed that the proanthocyanidins H2, H1 and K2 were also capable of causing a disruption/removal of monomeric Aβ 1-42.

### Example 6

### Disruption of Pre-Formed Aβ 1-42 and 1-40 Fibrils by Proanthocyanidin Compound H2 as Revealed by Circular Dichroism Spectroscopy

Circular dichroism (CD) spectroscopy is a method used to determine the effects of test compounds to disrupt pre-formed amyloid fibrils. In one study, as shown here circular dichroism spectroscopy was used to determine the effects of pure compound H2 (i.e. (epicatechin-4β→8-epicatechin) on disruption of β-pleated sheet structure of pre-formed Aβ 1-42 and 1-40 fibrils of the types found in the brains of patients with Alzheimer's and related disorders. For this study, Aβ 1-42 or Aβ 1-40 peptides (Bachem Inc., Torrance, Calif.) were first dissolved in 2 mM NaOH solution, maintaining the pH of these solutions above 10. The peptides were then dissolved in PBS containing 10% TFE, and the pH was adjusted to 7.2, Aβ 1-40 or Aβ 1-42 was incubated in the absence or presence of compound H2 at an Aβ:H2 weight/weight ratio of 1:0.1 (i.e. molar ratio of 1:1). At 3 and 7 days following incubation, CD spectra were recorded on a A VIV 202 spectropolarimeter with 50 μM of Aβ and H2 compound mixtures, All spectra were collected with 0.1 cm quartz cell using a thermstated cuvette holder. Wavelength traces were scanned from 260-195 nm at 0.5 nm increments with a bandwidth of 1 nm and averaged over a time of 5 seconds; the temperature was held constant at 25° C. All spectra reported are an average of 4 scans.

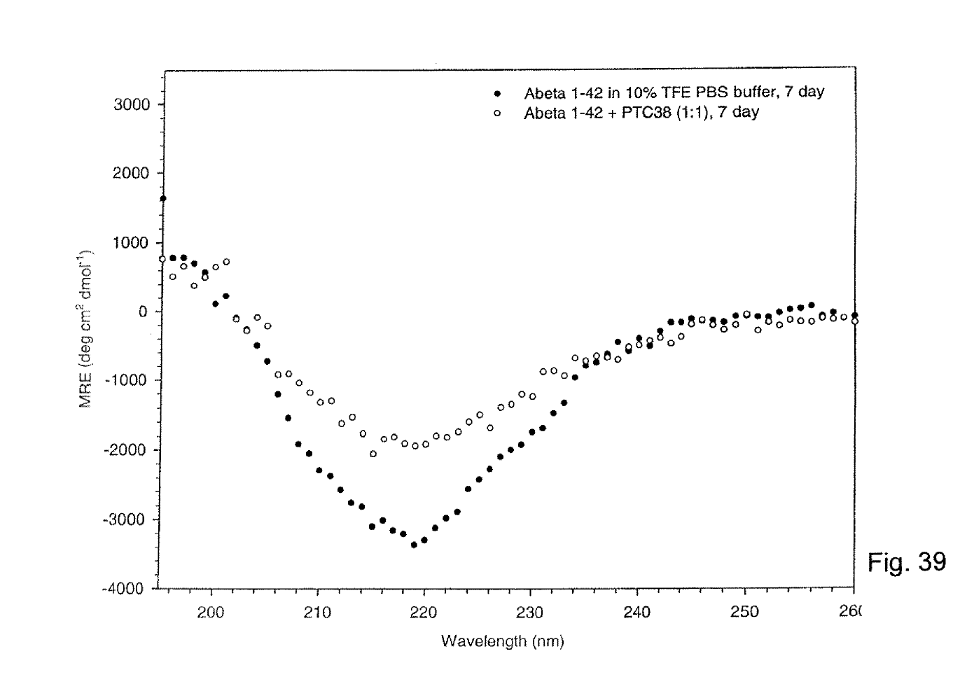

As shown in FIG. 39, Aβ 1-42 alone in 10% TFE PBS buffer showed the typical CD spectra of an amyloid protein with significant β-sheet structure, as demonstrated by the sharp minima observed at 218 nm. However, in the presence of H2 compound (at a 1:1 molar ratio) (noted as PTC38 in FIG. 39), a marked disruption of β-sheet structure in Aβ 1-42 fibrils was evident (with a significant increase in random coil or α-helix) as shown by the flattening out of the minima observed at 218 nm (compare to Aβ 1-42 alone)(FIG. 39). This was observed at both 3 (not shown) and 7 days (FIG. 39) following co-incubation of Aβ 1-42 fibrils with H2. This study clearly demonstrated that H2 compound had the ability to disrupt/disassemble the beta-pleated sheet structure characteristic of Aβ 1-42 fibrils.

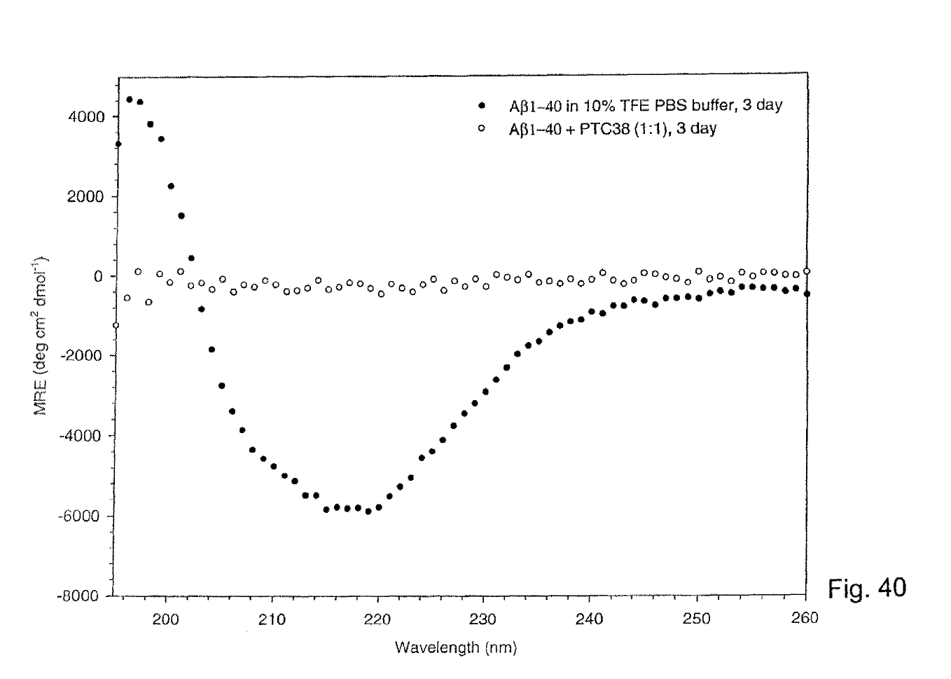

As shown in FIG. 40, Aβ 1-40 alone in 10% TFE PBS buffer also showed the typical CD spectra of an amyloid protein with significant α-sheet structure, as demonstrated by the sharp minima observed at 218 nm. However, in the presence of H2 compound (at a 1:1 molar ratio)(noted as PTC38 in FIG. 40), a nearly complete disruption/disassembly of β-sheet structure in Aβ 140 fibrils was evident (with a significant increase in random coil or α-helix) as shown by the complete flattening out of the minima observed at 218 nm (compare to Aβ 1-40 alone)(FIG. 40). This was observed at both 3 (not shown) and 7 days (FIG. 40) following co-incubation of Aβ 1-40 fibrils with H2. This study clearly demonstrated that H2 compound had the ability to disrupt/disassemble the beta-pleated sheet structure characteristic of Aβ 1-40 fibrils. Both Aβ 1-42 and Aβ 1-40 are known to be present in the amyloid deposits in the brains of patients with Alzheimer's disease and related disorders. This study confirms the efficacy of proanthocyanidins (in this particular case the epicatechin-epicatechin dimer) as potent inhibiting/disrupting agents of amyloid fibrils, and confirms the previous examples using Thioflavin T fluorometry and SDS-PAGE/ECL type assays that compound H2 and the other proanthocyanidins are potent anti-amyloid agents.

### Example 7

### Efficacy of Proanthocyanidins H2, H1 and K2 as Disruptors of α-Synuclein/NAC Fibrils

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 198; Arai et al., Neurosc., Lett. 259:83-86, 1999), a 140-amino acid protein (Ueda et al., Proc. Nat. Acad. Sci. U.S.A. 90:11282-11286, 1993), α-Synuclein recombinant protein, and non-amyloid component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. U.S.A 90:11282-11286, 1993). Inhibition, disruption/disassembly of preformed α-synuclein and/or NAC fibrils, are believed to serve as future therapeutics for the treatment of Parkinson's and Lewy body disease.

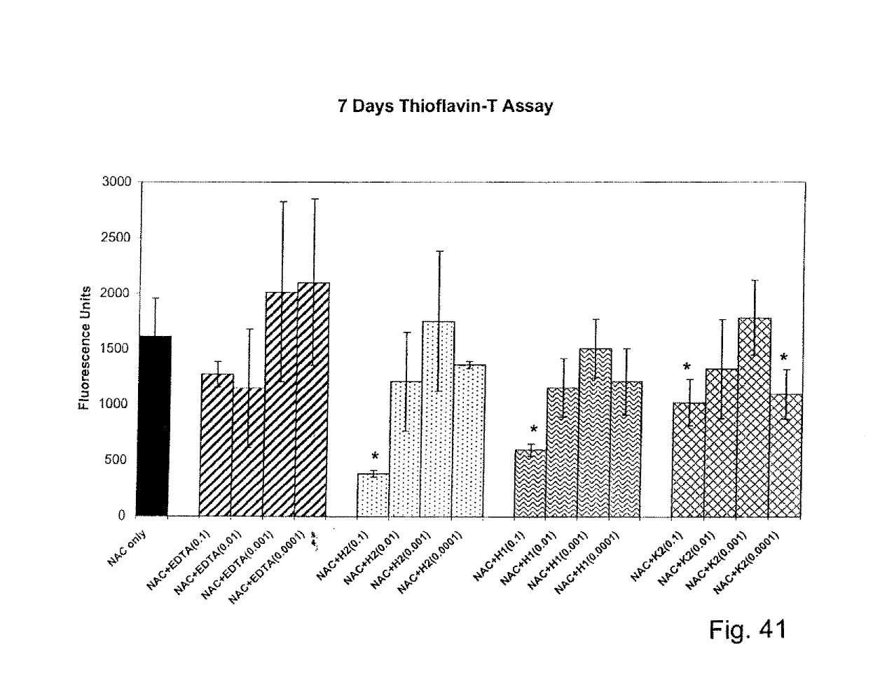

In the next study, we therefore determined the efficacy of proanthocyanidins, specifically H2, H1 and K2 as disruptor of pre-formed NAC fibrils. Thioflavin T fluorometry was used to determine the effects of H2, H1, K2 and EDTA (as a negative control) on disassembly/dissolution of pre-formed NAC fibrils (FIG. 41). In this study, 25 μM of pre-fibrillized NAC (Bachem Inc) was incubated at 37° C. for 1 week either alone, or in the presence of EDTA, H2, H1, or K2 at an NAC:test compound weight ratios of 1:0.1, 1:0.01, 1:0.001 or 1:0.0001. Following 3 or 7 days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer (pH 6.8). The fluorescence was read at 485 nm (444 nm excitation wavelength) using an ELISA plate fluorometer after subtraction of buffer alone as blank.

The results of day 7 incubations are presented here (FIG. 41), but similar results were obtained as early as 3 days. As shown in FIG. 41 whereas EDTA caused no significant inhibition of NAC fibrils at all concentrations tested, compound H2 caused a dose-dependent disruption/disassembly of preformed NAC fibrils, with a significant (p<0.01) 27+/−27% disruption when used at a NAC:H2 wt/wt ratio of 1:0.01; and a significant (p<0.01) 77+/−2% disruption when used at a NAC:H2 wt/wt ratio of 1:0.1 (i.e. 1:1 molar ratio). Similarly, compound H1 caused a dose-dependent disruption/disassembly of preformed NAC fibrils, with a significant 31+/−16% disruption when used at a NAC:H1 wt/wt ratio of 1:0.01; and a significant 64+/−3% disruption when used at a NAC:H1 wt/wt ratio of 0.1 (i.e. 1:1 molar ratio). Compound K2 also caused a dose-dependent disruption/disassembly of preformed NAC fibrils, with a significant 20+/−27% disruption when used at an NAC:K2 wt/wt ratio of 1:0.01; and a significant 39+/−12% disruption when used at an NAC:K2 wt/wt ratio of 1:0.1 (i.e. 1:1 molar ratio). This study indicated that the proanthocyanidins H2, H1 and K2 were also potent disruptors of NAC fibrils, and exerted their effect in a dose-dependent manner. It is expected that similar efficacy of these proanthocyanidins will be also observed for disruption/disassembly of α-synuclein fibrils.

### Example 8

### Efficacy of Proanthocyanidins H2, H1 and K2 as Disruptors of Type 2 Diabetes Amyloid Fibrils

Islet amyloid deposits are observed in ˜90% of patients with well-established type 2 diabetes and would appear to be a characteristic feature of the disease process (Westermark, J. Med. Sci. 77:91-94, 1972; Clark et al, Diabetes Res. 9:151-159, 1988). In many patients, the deposits are widespread and affect many islets. The degree of islet (predominantly β-cell) mass that has been replaced by amyloid may be a marker for the severity of the diabetic disease process, with those individuals requiring insulin treatment having the greatest islet mass reduction and amyloid formation (Westermark, Amyloid: Int. J. Exp. Clin. Invest. 1:47-60, 1994).

The major protein in type 2 diabetes islet amyloid is a 37-amino acid peptide known as islet amyloid polypeptide (IAPP) or amylin. IAPP is a known normal secretory product of the pancreatic β-cells (Kanh et al, Diabetes 39:634-638, 1990) that is stored in insulin-bearing cytoplasmic granules (Clark et al, Cell Tissue Res. 257:179-185, 1989). IAPP has been hypothesized to have an important role in the pathogenesis of type 2 diabetes through its impairment of β-cell function and reduction of β-cell mass (Johnson et al, N. Eng. J. Med. 321:513-518, 1989). Besides being able to form islet amyloid deposits that replace 8-cell mass, amyloid fibrils appear to damage islets directly. Studies as a whole suggest that islet amyloid formation plays a central role in the development of β-cell failure of type 2 diabetes. Therefore, agents or compounds able to inhibit or disrupt islet amyloid (i.e. IAPP) formation, deposition, accumulation or persistence, and/or cause a disruption/dissolution or disassembly of pre-formed IAPP fibrils are believed to lead to the discovery of new therapeutic compounds for the treatment of type 2 diabetes.

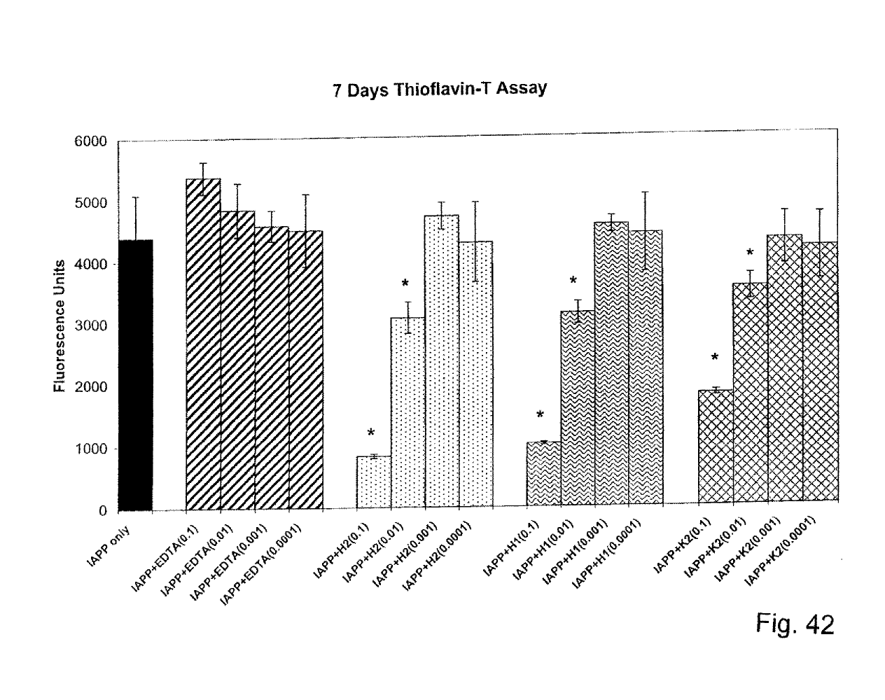

Therefore in the next study, we determined the efficacy of the proanthocyanidins, H2, H1 and K2 as disruptors/causing disassembly of pre-formed IAPP fibrils. Thioflavin T fluorometry was used to determine the effects of H2, H1, K2 and EDTA (as a negative control) on disassembly/dissolution of pre-formed IAPP fibrils (FIG. 42). In this study, 25 μM of pre-fibrillized IAPP (Bachem Inc) was incubated at 37° C. for 1 week either alone, or in the presence of EDTA, H2, H1, or K2 at an IAPP:test compound weight ratios of 1:0.1, 1:001, 1:0.001 or 1:0.0001. Following 3 or 7 days of co-incubation, 50 μl of each incubation mixture was transferred into a 96-well microtiter plate containing 1500 of distilled water and 50 μl of a Thioflavin T solution (i.e. 500 mM Thioflavin T in 250 mM phosphate buffer (pH 6.8). The fluorescence was read at 485 nm (444 nm excitation wavelength) using an ELISA plate fluorometer after subtraction of buffer alone as blank. The results of day 7 incubations are presented here (FIG. 42), but similar results were obtained as early as 3 days. As shown in FIG. 42 whereas EDTA caused no significant inhibition of IAPP fibrils at all concentrations tested, compound H2 caused a dose-dependent disruption/disassembly of preformed IAPP fibrils, with a significant (p<0.01) 36+/−5% disruption when used at a IAPP:H2 wt/wt ratio of 1:0.01; and a significant (p<0.01) 83+/−1% disruption when used at a IAPP:H2 wt/wt ratio of 1:0.1 (i.e. 1:1 molar ratio). Similarly, compound H1 caused a dose-dependent disruption/disassembly of preformed IAPP fibrils, with a significant 35+/−4% disruption when used at a IAPP:H1 wt/wt ratio of 1:0.01; and a significant 79+/−1% disruption when used at a IAPP:H1 wt/wt ratio of 0.1 (i.e. 1:1 molar ratio). Compound K2 also caused a dose-dependent disruption/disassembly of preformed IAPP fibrils, with a significant 26+/−4% disruption when used at an IAPP:K2 wt/wt ratio of 1:0.01; and a significant 62+/−1% disruption when used at an IAPP:K2 wt/wt ratio of 1:0.1 (i.e. 1:1 molar ratio). This study indicated that the proanthocyanidins H2, H1 and K2 were also potent disruptors of IAPP fibrils, and exerted their effect in a dose-dependent manner. This study also indicates that proanthocyanidins are expected to be useful for the treatment of IAPP amyloidosis in type 2 diabetes.

### Example 9

### Isolation and Identification of Peak K1 from PTI-777

### General Experimental Procedures:

A sample of the PTI-777 extract (1 gram) was dissolved in ethanol (2 ml) and then loaded onto a sephadex LH20 (10 g) column, prepared in ethanol. Elution of this column with ethanol (100 ml), followed by 5% acetone in ethanol (400 ml), 190% acetone in ethanol (200 ml), then 50% acetone in methanol (200 ml) gave 120 (12 ml fractions).

TABLE 5
Sephadex LH20 Column Fractionation of PTI-777

| Solvent | Fractions | Peaks present | Weight |
|---|---|---|---|
| EtoH | 25-29 | other, J | 4 mg |
| | 30-35 | J, other | 36 mg |
| | 36-37 | J, and K1 | 5 mg |
| | 38-42 | K1 | 22 mg |
| | 43-48 | K1, H2, other | 13 mg |
| | 49-56 | H2 | 89 mg |
| | 57-71 | H2, H1 | 89 mg |
| | 72-83 | H1 | 38 mg |
| 5% acetone in ethanol | 84-90 | H1, other | 23 mg |
| | 91-92 | other, H1, K2 | 7 mg |
| 10% acetone in ethanol | 93-100 | K2 | 40 mg |
| 50% acetone in ethanol | 101-114 | mix | 92 mg |
| 50% acetone in methanol | 115-119 | mix + post-L | 400 mg |
| 100% methanol | 120-end | post-L only | 187 mg |

The analytical HPLC conditions for monitoring of K1 and the K1 acetate (described below) are under the same conditions as in Example 2, where method 1 was used.

### Isolation of Peak K1:

Fractions 38 to 42 (see Table 5 above) contained compound R1 (22 mg) as a pale brown gum. The retention time of this R1 peak was 15.0 minutes as monitored by HPLC Method 1.

For acetylation of K1 to help determine the structure, a sample of K1 (15 mg) was dissolved in a mixture of acetic anhydride (0.5 ml) and pyridine (0.5 ml). The mixture stood at room temperature for 18 hours, then the solvents were removed in vacuo to give the K1 peracetate (16 mg) as a colourless gum. The NMR data is shown in Table 6 below.

### Identification of K1 and the K1 Peracetate:

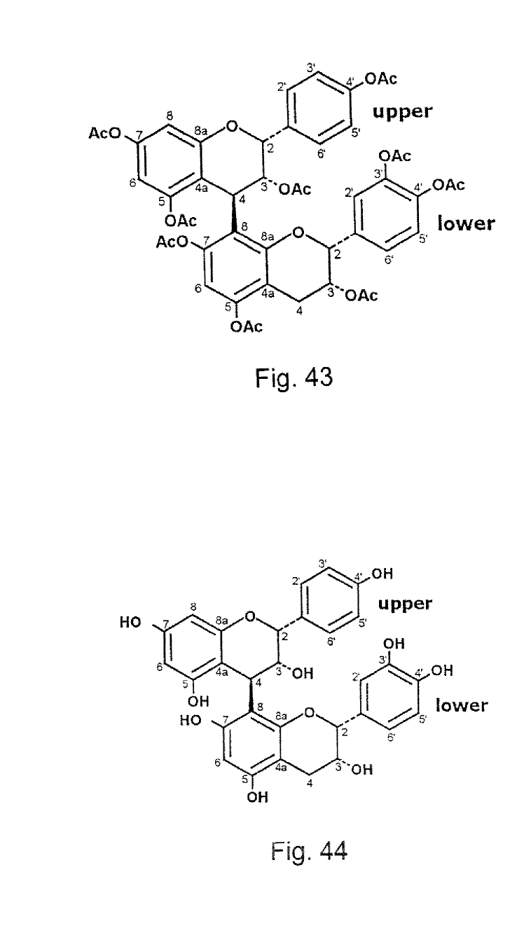

The minor component of peak K, called K1, of the PTI-777 extract was isolated by column chromatography over sephadex LH20, monitored by HPLC. Elution with 95% ethanol followed by increasing amounts of acetone and water, followed by methanol, gave pure peak K1 in fractions 38 to 42 (see Table 5). The structure of the K1 peracetate is shown in FIG. 43, whereas the structure of K1 is shown in FIG. 44. To arrive at these structures, the following analysis and results were obtained.

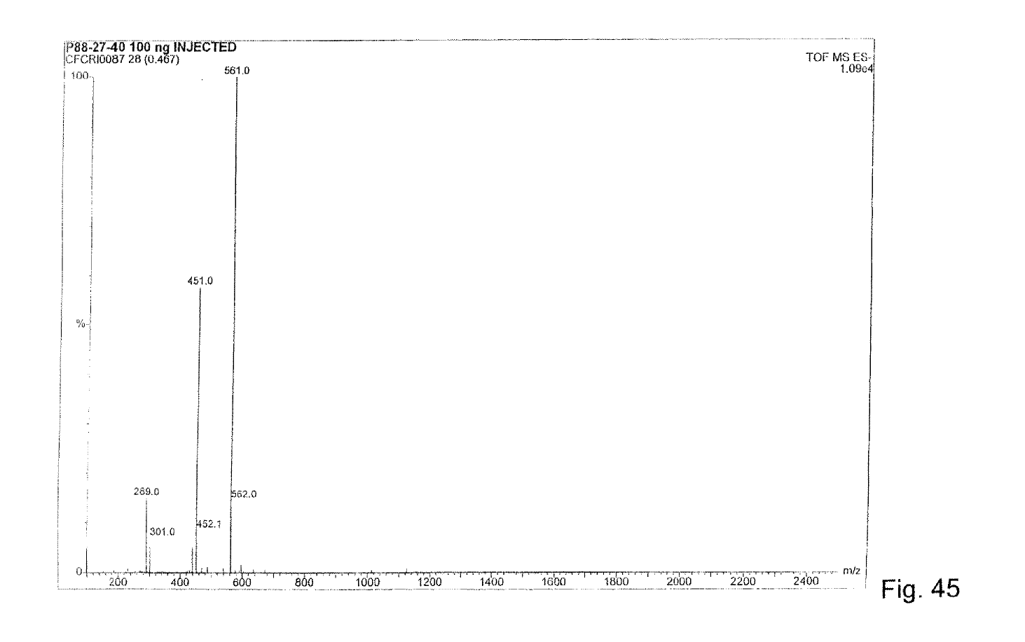

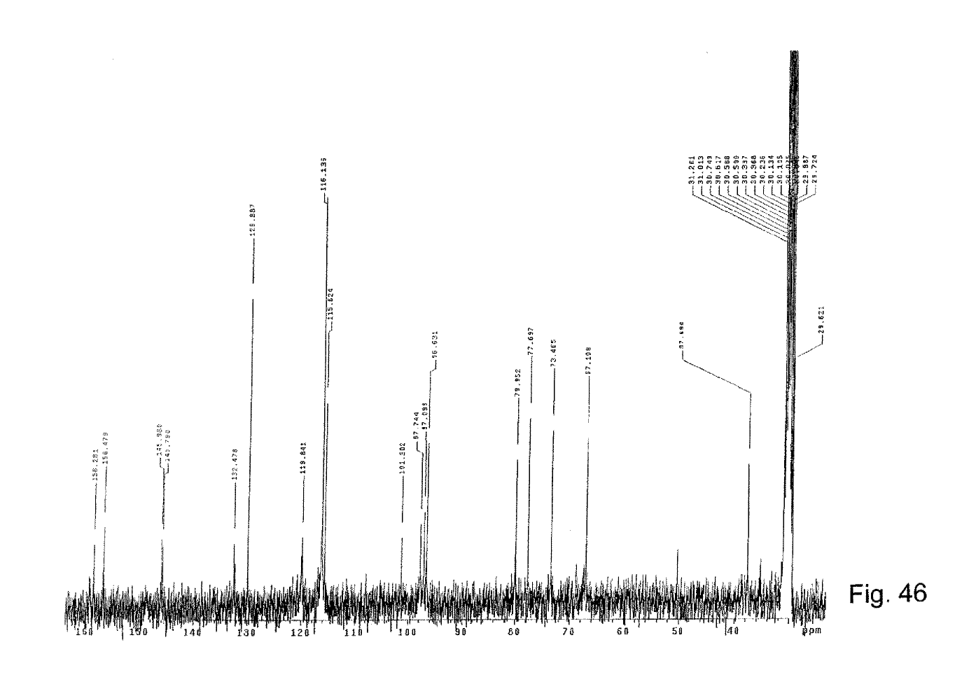

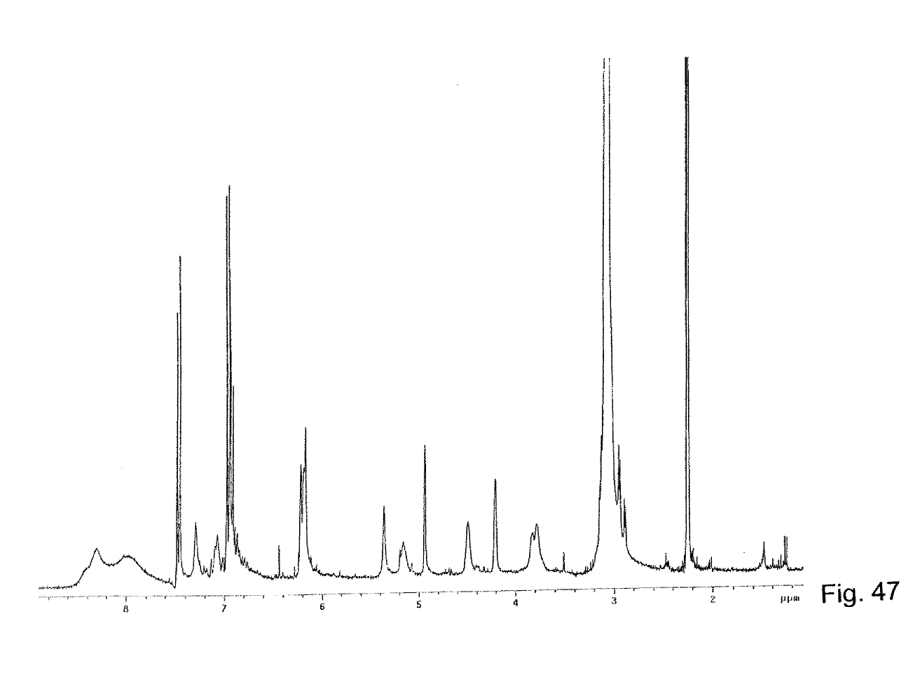

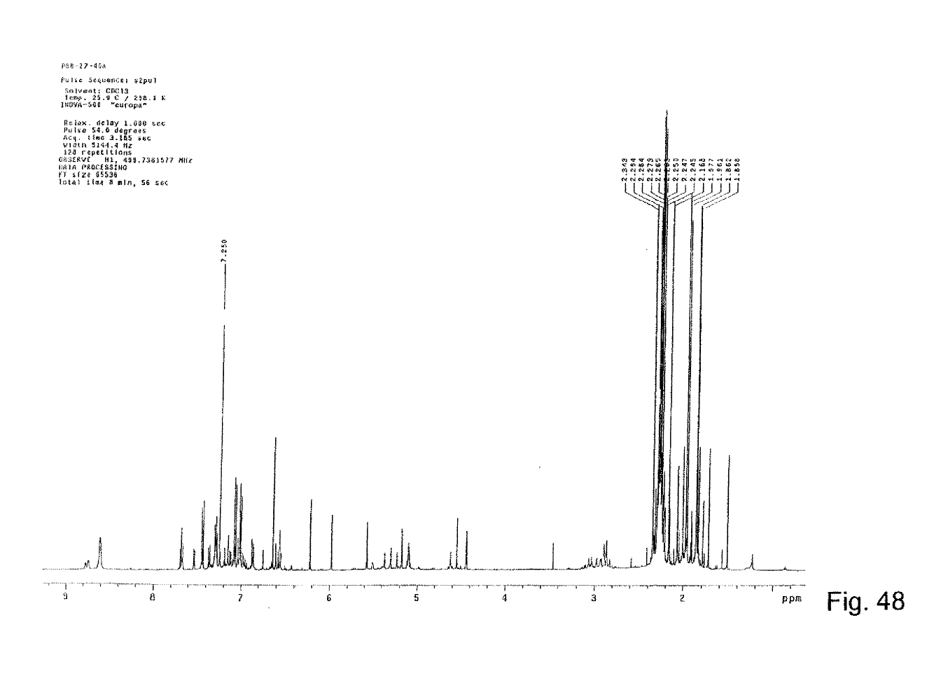

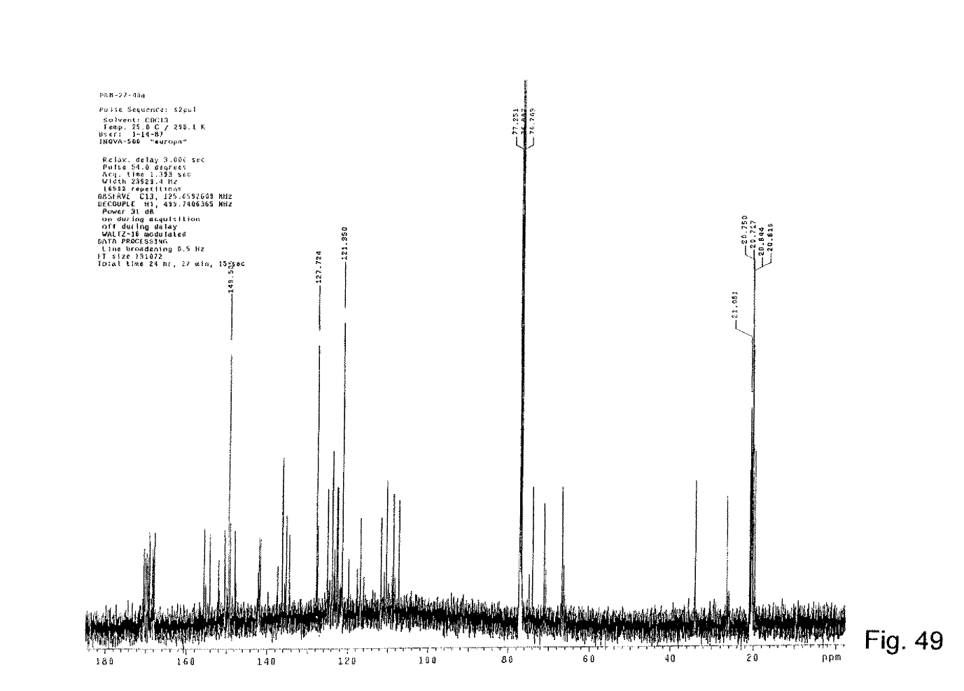

A ⁻ve ion electrospray mass spectrum of K1 gave a clean 100% ion at 561 daltons (FIG. 45). This is appropriate for the molecular ion (M⁺−H) of a molecular formula of C₁₁H₂₉O₁₁ (molecular weight=562), such as a mixed dimer of one epicatechin, or isomeric unit and one epiafzelechin, or isomeric unit. The ¹³C NMR of K1 showed signals consistent with some kind of flavanol dimer (FIG. 46). The ¹H NMR of K1 showed there to be similar broad peaks to that seen in compound H2 (FIG. 47), so it was decided to acetylate the compound to determine the final structure. Acetylation of a sample of pure K1 gave a peracetate (structure shown in FIG. 43). The ¹H and ¹³C NMR spectrum of the K1 peracetate are shown in FIGS. 48 and 49, respectively. Two sets of signals were seen in both the ¹H and ¹³C NMR spectra, in a ratio of three to one. These were due to rotational isomers (atropisomers). We solved the structure using the signals of the major atropisomer (see Table 6 below).

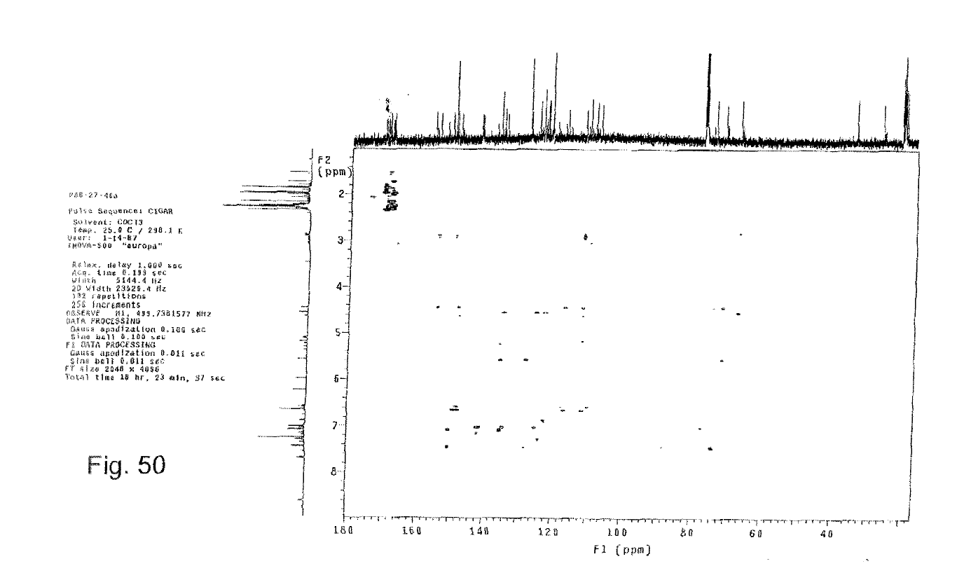

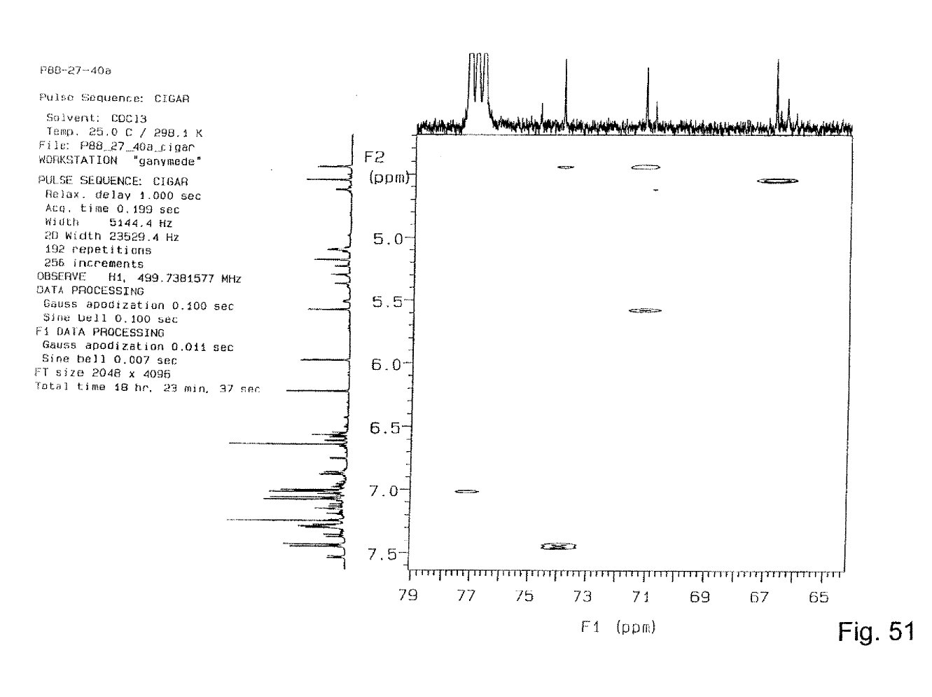

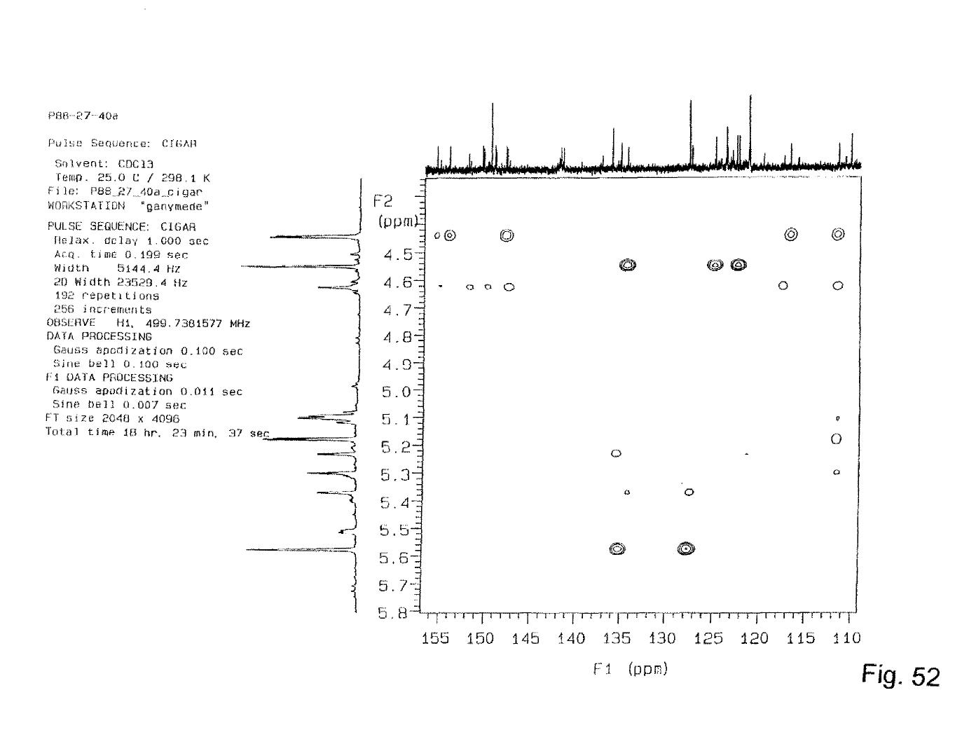

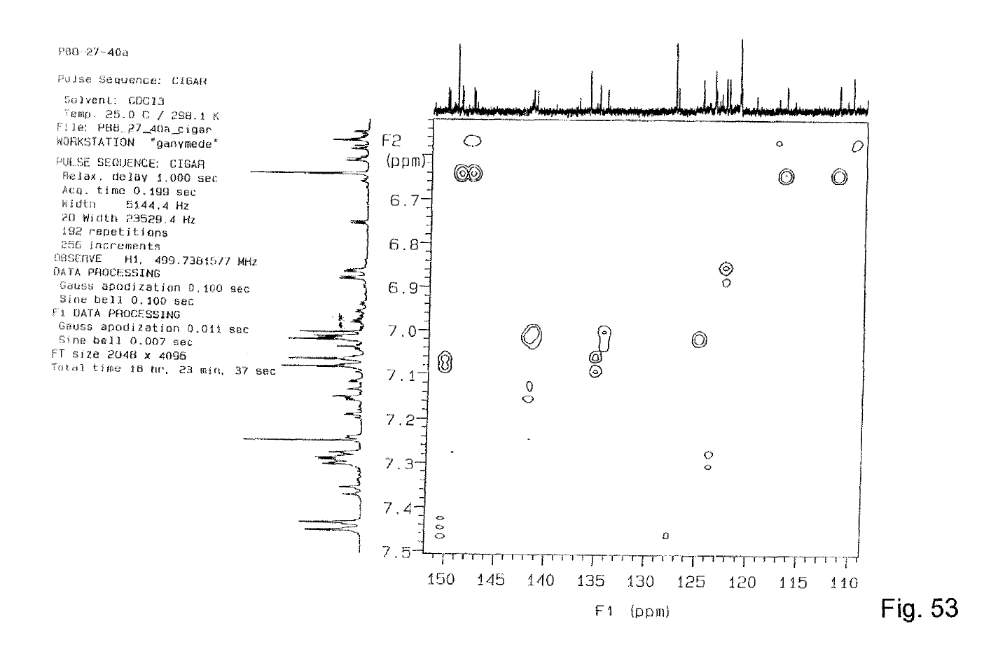

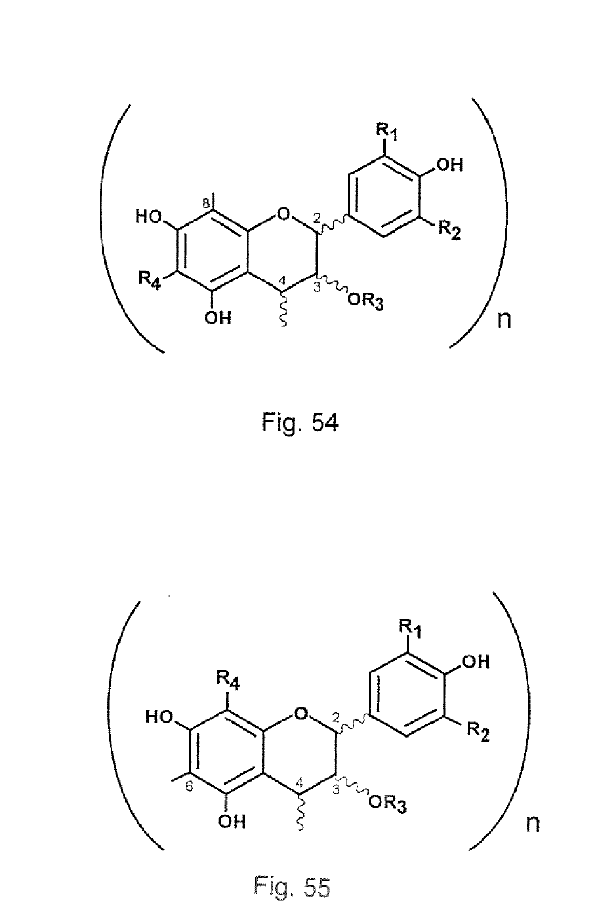

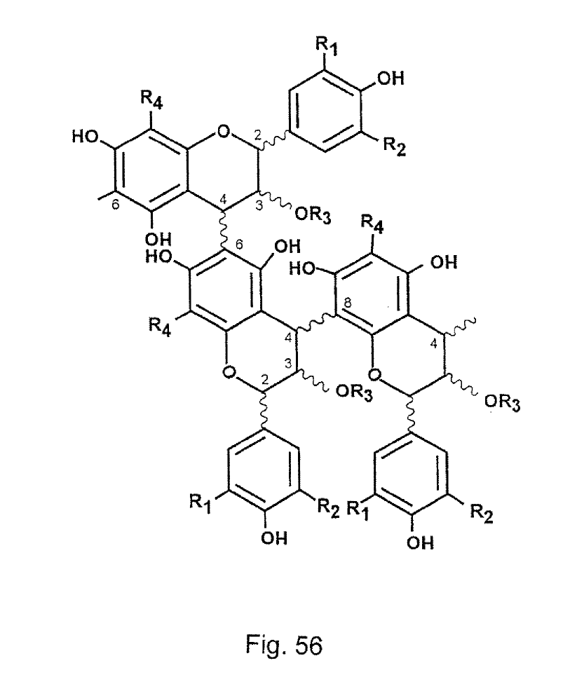

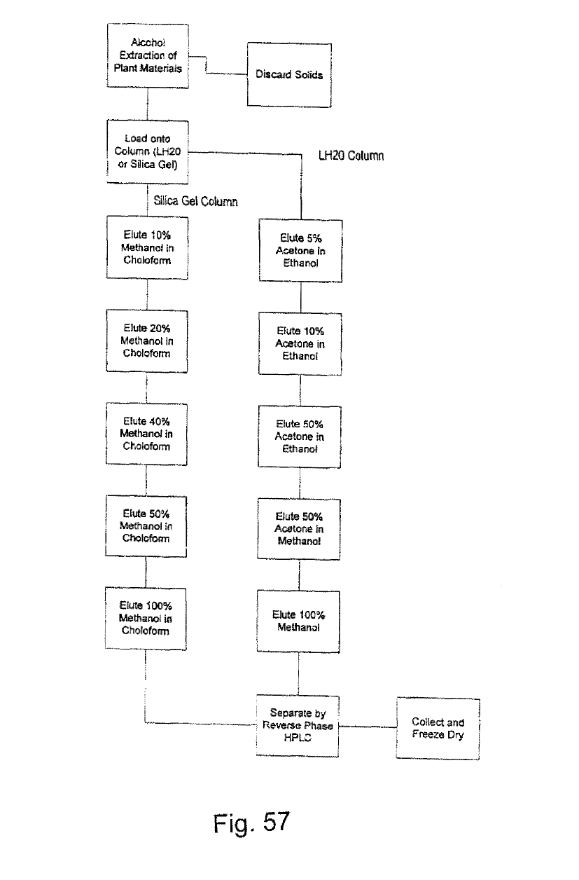

The presence of two flavan-3-ol units could be seen from the four ¹³C signals for the C-2 and C-3 positions in the 60-80 region, as well as a signal at 26.61 for the free C-4 position of the lower unit and a signal at 34.14 for the linked C-4 of the upper unit. A CIGAR ¹H-¹³C correlation experiment (FIGS. 50-53) showed that the two units were connected from the 4(u) position to the 8(l) position, by the correlation between H-4(u) and C-8(l). The stereochemistries at C-2 and C-3 of both upper and lower units was shown to be the same as in epicatechin by the similar chemical shifts of the ¹H and ¹³C signals for the lower unit, as well as the similar low coupling constants between H-2 and H-3 in both units. The lower flavan-3-ol unit was shown to be epicatechin by CIGAR correlations from H-2(l) to C-2′ and C-6′ signals of a 3′,4′-dioxygenated aromatic ring. The upper flavan-3-ol unit was identified by CIGAR correlations from H-2(u) to equivalent C-2′/C-6′ signals of a 4′-oxygenated ring. This constitutes an epiafzelechin unit. The structure of the natural product K1 was therefore assigned to be epiafzelechin-4β→8-epicatechin. This compound is a known compound (Kashiwada et al, Chem. Pharma. Bull. 36:3947, 1988; Morimoto et al, Chem. Pharm. Bull. 34:888-893, 1990). Our NMR data on the structure for K1 matched partial NMR data published on epiafzelechin-4β→8-epicatechin. The optical rotation of −1.4° compared to a literature value of +29° showed uncertain absolute stereochemistry.

### Peak K1 Data Summary

−ve electrospray mass spectroscopy 561 (M⁺−H, 100%)

molecular weight 562

¹H NMR ((CD₃)₂CO):2.88 (2H, m, H-4l), 3.59 (1H, br s, OH), 3.73 (1H, br s, OH), 4.11 (1H, s), 4.39 (1H, br s), 4.84 (1H, s H-4u), 5.07 (1H, br s), 5.26 (1H, s), 6.08 (1H, s), 6.09 (1H, s), 6.12 (1H, s), 6.80 (1H, m), 6.85 (2H, d, J=8 Hz), 6.97 (1H, m), 7.19 (1H, br s), 7.37 (2H, d, J=8 Hz) and 7.40-8.20 (7H, br s, OHs)

¹³C NMR ((CD₃)₂CO): 37.69, 67.11, 73.47, 77.70, 79.95, 96.63, 97.10, 97.74, 101.30, 115.62, 116.14, 119.84, 129.89, 132.48, 145.79, 145.98, 156.78 and 158.28.

UV (MeOH) λ max (log ε) 216 (4.89), 227 sh (4.74) and 280 (4.04) nm;

[α]²⁴₅₈₉ₙₘ−1.4°, [α]²⁴₅₇₇ₙₘ−23.1°, [α]²⁴₅₄₆ₙₘ−62.3°, (c 0.1, MeOH).

TABLE 6
500 MHz NMR data of the K1 Peracetate in Deuterochloroform,
Major Atropisomer.

| | C^{a} | H^{b} | CIGAR^{c} H-C |
|---|---|---|---|
| upper | | | |
| 2 | 73.96 | 5.58 s | 3u, 1′u, 2′u/6′u |
| 3 | 71.18 | 5.18 dd 1, 2 | none observed |
| 4 | 34.14 | 4.44 d2 | 2u, 3u, 4au, 5, 8au, 8l, (8al) |
| 4a | 111.66 | | |
| 5 | 147.81 | | |
| 6 | 108.52 | 6.22 d 2 | none observed |
| 7 | 149.04 | | |
| 8 | 107.23 | 5.98 d 2 | none observed |
| 8a | 154.15 | | |
| 1′ | 135.23 | | |
| 2′ | 127.72 | 7.44 d 8 | 2u, (4′u) |
| 3′ | 121.35 | 7.07 d 8 | 4′u |
| 4′ | 150.50 | | |
| 5′ | 121.35 | 7/07 d 8 | 4′ u |
| 6′ | 127.72 | 7.44 d 8 | 2u, (4′u) |
| lower | | | |
| 2 | 77.16 | 4.55s | 3 l, 1′l, 2′l, 6′l |
| 3 | 66.77 | 5.10 m | |
| 4 | 26.61 | 2.85 d 18 | 3l, 5 l, 4a l, 8a l |
| | | 2.91 dd 18, 5 | 3 l, 5 l, 4a l, 8a l |
| 4a | 111.59 | | |
| 5 | 147.91 | | |
| 6 | 110.27 | 6.64 s | (2 l), 4a l, 5 l, 7 l, 8 l |
| 7 | 149.12 | | |
| 8 | 116.79 | | 4au, 6u, 7u, 8au |
| 8a | 155.55 | | |
| 1′ | 134-48 | | |
| 2′ | 122.44 | 7.03 m | 2 l, 6′ l |
| 3′ | 141.59 | | |
| 4′ | 141.90 | | |
| 5′ | 122.71 | 7.04 m | 6′ l |
| 6′ | 124.98 | 6.87 dd 8, 2 | none observed |

^{a}shift in ppm.
^{b}Shift in ppm, multiplicity, couplings in Hz.
^{c}Brackets indicate weak correlations, l = lower unit, u = upper unit.
OAc groups not shown.

### Example 10

### Therapeutic Applications

Proanthocyanidins act as potent inhibitors/disruptors and/or causing disassembly of amyloid fibrils (regardless of the type of amyloid protein present. Examples are shown for Aβ, NAC and IAPP fibrils), as well as a potent inhibitor/disruptor of α-synuclein/NAC fibrils. Both procyanidin dimers and trimers are shown specifically to inhibit such fibrillogenesis, and our ongoing studies suggest that procyanidin tetramers and oligomers (greater than tetramers) are also able to exert such amyloid fibril inhibiting effects. Thus, preferred therapeutic applications include the use of proanthocyanidins and procyanidins for the treatment of amyloid diseases, and diseases which include α-synuclein/NAC fibrillogenesis.

The proanthocyanidins of the present invention were discovered, isolated and identified from the plant Uncaria tomentosa. However, it is probable that similar amyloid/α-synuclein/NAC inhibitory activity is observed with any proanthocyanidin regardless of the source (i.e. plant or food), and will include proanthocyanidins that can be synthesized by methods known to those knowledgeable and skilled in the art.

Preparations of proanthocyanidins compounds for parenteral administration include sterile aqueous or non-aqueous solutions, suspensions, emulsions, which may contain axillary agents or excipients which are known in the art. Pharmaceutical or pharmacological compositions such as tablets, pills, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectible solutions, sterile packaged powders, can be prepared according to routine method and are known in the art.

Proanthocyanidins of the present invention may be administered by any means that achieve their intended purpose, for example to treat amyloid diseases, such as Alzheimer's disease or type 2 diabetes, or other pathologies involving α-synuclein/NAC fibrillogenesis, using a proanthocyanidin described herein, in the form of a pharmaceutical or pharmacological composition.

For example, administration of such a composition may be by various parenteral routes such as subcutaneous, intravenous, intradermal, intramuscular, intraperitoneal, intranasal, transdermal or buccal routes. Alternatively, or concurrently, administration may be by the oral route. Parenteral administration can be by bolus injection or by gradual perfusion over time. A preferred mode of using a proanthocyanidin pharmaceutical composition of the present invention is by oral administration or intravenous application.

A typical regimen for preventing, suppressing or treating amyloid pathologies, such as Alzheimer's disease amyloidosis, comprises administration of an effective amount of a proanthocyanidin over a period of one or several days, up to and including between one week to about 10 years.

It is understood that the dosage of the proanthocyanidin of the present invention administered in vivo or in vitro will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

The total dose required for each treatment may be administered by multiple doses or in a single dose. A proanthocyanidin or procyanidin compound may be administered alone or in conjunction with other therapeutics directed to amyloid disease or α-synuclein/NAC fibrillogenesis, such as Alzheimer's disease or Parkinson's disease, as described herein. Effective amounts of a proanthocyanidin compound for treatment, are about 10 mg to about 1,000 mg/kg body weight, and preferably from about 10 mg to 100 mg/kg body weight, such as 10, 20, 30, 40, 50, 60, 70, 80, 90 or 100 mg/kg body weight.

Preparations for parenteral administration include sterile aqueous or non-aqueous solutions, suspensions and emulsions, which may contain axillary agents or excipients that are known in the art. Pharmaceutical compositions containing a proanthocyanidin of the present invention may include all compositions where the proanthocyanidin is contained in an amount effective to achieve its intended purpose. In addition, a pharmaceutical composition may contain suitable pharmaceutically acceptable carriers, such as excipients, carriers and/or auxiliaries that facilitate processing of the active compounds into preparations which can be used pharmaceutically.

Pharmaceutical compositions comprise at least one proanthocyanidin compound may also include solutions for administration intravenously, subcutaneously, dermally, orally, mucosally, rectally, or may be by injection or orally, and contain from about 0.01 to 100%, preferably about 95-100% of active compound together with the excipient. Pharmaceutical compositions for oral administration include pills, tablets, caplets, soft and hard gelatin capsules, lozenges, sachets, cachets, vegicaps, liquid drops, elixers, suspensions, emulsions, solutions and syrups.

The proanthocyanidin compounds for Alzheimer's and Parkinson's disease, and other central nervous system disorders may be optimized to cross the blood-brain barrier. Methods of introductions include but are not limited to systemic administration, parenteral administration, i.e. via an intraperitoneal, intravenous, perioral, subcutaneous, intramuscular, intraarterial, intradermal, intramuscular, intranasal, epidural or oral routes. In a preferred embodiment for the treatment of central nervous system disorders, a proanthocyanidin compound may be directly administered to the cerebrospinal fluid by intraventricular injection. In a specific embodiment, it may be desirable to administer a proanthocyanidin compound locally to the areas of tissue in need of treatment; this may be achieved by, for example, and not by way of limitation, local infusion during surgery, topical application, by injection, by infusing a cannulae with osmotic pump, by means of a catheter, by means of a suppository, or by means of an implant.

In yet another embodiment, a proanthocyanidin compound may be delivered in a control led release system, such as an osmotic pump. In yet another embodiment, a controlled release system can be placed in proximity to the therapeutic target, i.e. the brain, thus requiring only a fraction of the systemic dose.

### Example 11

### Clinical Testing in Alzheimer's Patients for Example

Five to fifty women are selected for a clinical study. The women are post-menopausal, i.e. have ceased menstruating for between 6 and 12 months prior to the study's initiation, have been diagnosed with early stage Alzheimer's disease, and expected to have worsening symptoms of Alzheimer's disease within the study period but are in good general health otherwise. The study has a placebo group, i.e. the women are divided into two groups, one of which receives the compound of this invention and the other receives a placebo. The patients are benchmarked as to memory, cognition, reasoning, and other symptoms associated with Alzheimer's disease. Women in the test group receive a therapeutic dose of the compound by the oral route. They continue this therapy for 6-36 months. Accurate records are kept as to the benchmarked symptoms in both groups and at the end of the study these results are compared. The results are compared both between members of each group and also the results for each patient are compared to the symptoms reported by each patient before the study began. Activity of the compound is illustrated by an attenuation of the typical cognitive decline and/or behavioral disruptions associated with Alzheimer's disease.

Utility of the compounds is evidenced by activity in at least one of the above assays.

### INDUSTRIAL APPLICABILITY

To date there is no suggested usage of proanthocyanidin compounds for treatment of amyloid diseases, amyloidoses, amyloid fibrillogenesis or diseases characterized by NAC or α-synuclein fibrillogenesis. It is believed that cost effective treatment for these conditions throughout the world is now at hand, and that this disclosure readily puts into the hands of health providers and health officials worldwide a means to alleviate much suffering and economic loss.

While this invention has been described in conjunction with specific embodiments and examples, it will be apparent to a person of ordinary skill in the art, having regard to this disclosure, that equivalents of the specifically disclosed materials and techniques will also be applicable to this invention; and such equivalents are intended to be included within the following claims.

1-56. (canceled)

57. A method for reducing an amount of β-amyloid protein in a mammal by administering a procyanidin to the mammal.

58. The method of claim 57, wherein the mammal is a human.

59. The method of claim 57, wherein the procyanidin is a dimer or trimer of epicatechin and/or catechin units.

60. The method of claim 59, wherein the procyanidin dimer is selected from the group consisting of procyanidin type B1, B2, B3, B4, B5, B6, B7, and B8.

61. The method of claim 59, wherein the procyanidin dimer is epicatechin-4β->8-epicatechin.

62. The method of claim 59, wherein the procyanidin dimer is catechin-4α->8-epicatechin.

63. The method of claim 59, wherein the procyanidin dimer is epiafzelechin-4β->8-epicatechin.

64. The method of claim 59, wherein the procyanidin trimer is epicatechin-4β->8-epicatechin-4β->8-epicatechin.

65. A method for improving cognition in a mammal by administering a procyanidin to the mammal.

66. The method of claim 65, wherein the mammal is a human.

67. The method of claim 65, wherein the procyanidin is a dimer or trimer of epicatechin and/or catechin units.

68. The method of claim 67, wherein the procyanidin dimer is selected from the group consisting of procyanidin type B1, B2, B3, B4, B5, B6, B7, and B8.

69. The method of claim 67, wherein the procyanidin dimer is epicatechin-4β->8-epicatechin.

70. The method of claim 67, wherein the procyanidin dimer is catechin-4-α->8-epicatechin.

71. The method of claim 67, wherein the procyanidin dimer is epiafzelechin-4β->8-epicatechin.

72. The method of claim 67, wherein the procyanidin trimer is epicatechin-4β->8-epicatechin-4β->8-epicatechin.

73. A method for improving memory in a mammal by administering a procyanidin to the mammal.

74. The method of claim 73, wherein the mammal is a human.

75. The method of claim 73, wherein the procyanidin is a dimer or trimer of epicatechin and/or catechin units.

76. The method of claim 73, wherein the procyanidin dimer is selected from the group consisting of procyanidin type B1, B2, B3, B4, B5, B6, B7, and B8.

77. The method of claim 76, wherein the procyanidin dimer is epicatechin-4β->8-epicatechin.

78. The method of claim 76, wherein the procyanidin dimer is catechin-4α->8-epicatechin.

79. The method of claim 76, wherein the procyanidin dimer is epiafzelechin-4β->8-epicatechin.

80. The method of claim 76, wherein the procyanidin trimer is epicatechin-4β->8-epicatechin-4β->8-epicatechin.
